(19) United States
(12) Patent Application Publication
(10) Pub. No.: US 2003/0077246 A1
(43) Pub. Date: Apr. 24, 2003

(54) TNFr/OPG-like molecules and uses thereof

(76) Inventors: Andrew A. Welcher, Ventura, CA (US); Gary M. Fox, Newbury Park, CA (US); Michael J. Boedigheimer, Newbury Park, CA (US); Junyan Shu, Thousand Oaks, CA (US); Shuqian Jing, Thousand Oaks, CA (US); Brian D. Bennett, Thousand Oaks, CA (US); Roland Luethy, Camarillo, CA (US)

Correspondence Address: MARSHALL, GERSTEIN & BORUN, Thomas A. Cawley, Jr. Ph.D., Sears Tower, Suite 6300, 233 S. Wacker Drive, Chicago, IL, 60606-6357, US

(21) Appl. No.: 10/146,574

(22) Filed: May 15, 2002

Related U.S. Application Data
(62) Division of application No. 09/724,037, filed on Nov. 28, 2000.
(60) Provisional application No. 60/172,306, filed on Dec. 16, 1999.

(51) Int. Cl. C07K 14/715; A61K 38/19; C07H 21/04; C12P 21/02; C12N 5/06

(52) U.S. Cl. 424/85.1; 435/69.1; 435/320.1; 435/325; 530/350; 536/23.5

(57) ABSTRACT

The present invention provides novel TNFr/OPG-like polypeptides and nucleic acid molecules encoding the same. The invention also provides vectors, host cells, antibodies, and methods for producing TNFr/OPG-like polypeptides. Also provided for are methods for the diagnosis and treatment of diseases with TNFr/OPG-like polypeptides.

### RELATED APPLICATION

The present application claims priority under 35 U.S.C. §119 U.S. provisional patent application Serial No. 60/172,306 filed Dec. 16, 1999.

### FIELD OF THE INVENTION

The present invention relates to novel tumor necrosis factor receptor/osteoprotegerin-like (TNFr/OPG-like) polypeptides, and nucleic acid molecules encoding the same. The invention also relates to vectors, host cells, selective binding agents, such as antibodies, and methods for producing TNFr/OPG-like polypeptides. Also provided for are methods for the diagnosis and treatment of diseases associated with TNFr/OPG-like polypeptides.

### BACKGROUND OF THE INVENTION

Technical advances in the identification, cloning, expression and manipulation of nucleic acid molecules have greatly accelerated the discovery of novel therapeutics based upon deciphering of the human genome. Rapid nucleic acid sequencing techniques can now generate sequence information at unprecedented rates and, coupled with computational analyses, allow the assembly of overlapping sequences into entire genomes and the identification of polypeptide-encoding regions. A comparison of a predicted amino acid sequence against a database compilation of known amino acid sequences can allow one to determine the extent of homology to previously identified sequences and/or structural landmarks. The cloning and expression of a polypeptide-encoding region of a nucleic acid molecule provides a polypeptide product for structural and functional analyses. The manipulation of nucleic acid molecules and encoded polypeptides to create variants and derivatives thereof may confer advantageous properties on a product for use as a therapeutic.

In spite of the significant technical advances in genome research over the past decade, the potential for the development of novel therapeutics based on the human genome is still largely unrealized. Many genes encoding potentially beneficial polypeptide therapeutics, or those encoding polypeptides which may act as “targets” for therapeutic molecules, have still not been identified. In addition, structural and functional analyses of polypeptide products from many human genes have not been undertaken.

Accordingly, it is an object of the invention to identify novel polypeptides and nucleic acid molecules encoding the same, which have diagnostic or therapeutic benefit.

### SUMMARY OR THE INVENTION

The present invention relates to novel TNFr/OPG-like nucleic acid molecules and encoded polypeptides, and uses thereof.

The invention provides for an isolated nucleic acid molecule comprising a nucleotide sequence selected from the group consisting of:

(a) the nucleotide sequence set forth in SEQ ID NOS: 1 or 3;

(b) a nucleotide sequence encoding the polypeptide set forth in SEQ ID NO: 2 or SEQ ID NO: 4;

(c) a nucleotide sequence which hybridizes under moderately or highly stringent conditions to the complement of (a) or (b), wherein the encoded polypeptide has an activity of the polypeptide set forth in SEQ ID NO: 2 or SEQ ID NO: 4; and

(d) a nucleotide sequence complementary to any of (a) through (c).

The invention also provides for an isolated nucleic acid molecule comprising a nucleotide sequence selected from the group consisting of:

(a) a nucleotide sequence encoding a polypeptide that is at least about 70, 75, 80, 85, 90, 95, 96, 97, 98, or 99 percent identical to the polypeptide set forth in SEQ ID NO: 2 of SEQ ID NO: 4, wherein the polypeptide has an activity of the encoded polypeptide set forth in SEQ ID NO: 2 or SEQ ID NO: 4 as determined using a computer program selected from the group consisting of GAP, BLASTP, BLASTN, FASTA, BLASTA, BLASTX, BestFit, and the Smith-Waterman algorithm;

(b) a nucleotide sequence encoding an allelic variant or splice variant of the nucleotide sequence set forth in SEQ ID NOS: 1 or 3, wherein the encoded polypeptide has an activity of the polypeptide set forth in SEQ ID NO: 2 or SEQ ID NO: 4;

(c) a nucleotide sequence of SEQ ID NOS: 1 OR 3, (a), or (b) encoding a polypeptide fragment of at least about 25 amino acid residues, wherein the polypeptide has an activity of the polypeptide set forth in SEQ ID NO: 2 or SEQ ID NO: 4;

(d) a nucleotide sequence encoding a polypeptide that has a substitution and/or deletion of 1 to 430 amino acid residues set forth in SEQ ID NO: 1 or 1 to 436 amino acid residues of SEQ ID NO: 3 wherein the encoded polypeptide has an activity of the polypeptide set forth in SEQ ID NO: 2 or SEQ ID NO: 4;

(e) a nucleotide sequence of SEQ ID NOS: 1 or 3 , or (a)-(d) comprising a fragment of at least about 16 nucleotides;

(f) a nucleotide sequence which hybridizes under moderately or highly stringent conditions to the complement of any of (a)-(e), wherein the encoded polypeptide has an activity of the polypeptide set forth in SEQ ID NO: 2 or SEQ ID NO: 4; and

(g) a nucleotide sequence complementary to any of (a)-(e).

The invention further provides for an isolated nucleic acid molecule comprising a nucleotide sequence selected from the group consisting of:

(a) a nucleotide sequence encoding a polypeptide set forth in SEQ ID NO: 2 or SEQ ID NO: 4 with at least one conservative amino acid substitution, wherein the encoded polypeptide has an activity of the polypeptide set forth in SEQ ID NO: 2 or SEQ ID NO: 4;

(b) a nucleotide sequence encoding a polypeptide set forth in SEQ ID NO: 2 or SEQ ID NO: 4 with at least one amino acid insertion, wherein the encoded polypeptide has an activity of the polypeptide set forth in SEQ ID NO: 2 or SEQ ID NO: 4;

(c) a nucleotide sequence encoding a polypeptide set forth in SEQ ID NO: 2 or SEQ ID NO: 4 with at least one amino acid deletion, wherein the encoded polypeptide has an activity of the polypeptide set forth in SEQ ID NO: 2 or SEQ ID NO: 4;

(d) a nucleotide sequence encoding a polypeptide set forth in SEQ ID NOS: 2 or 4 which has a C- and/or N-terminal truncation, wherein the encoded polypeptide has an activity of the polypeptide set forth in SEQ ID NO: 2 or SEQ ID NO: 4;

(e) a nucleotide sequence encoding a polypeptide set forth in SEQ ID NO: 2 or SEQ ID NO: 4 with at least one modification selected from the group consisting of amino acid substitutions, amino acid insertions, amino acid deletions, C-terminal truncation, and N-terminal truncation, wherein the polypeptide has an activity of the encoded polypeptide set forth in SEQ ID NO: 2 or SEQ ID NO: 4;

(f) a nucleotide sequence of (a)-(e) comprising a fragment of at least about 16 nucleotides;

(g) a nucleotide sequence which hybridizes under moderately or highly stringent conditions to the complement of any of (a)-(f), wherein the encoded polypeptide has an activity of the polypeptide set forth in SEQ ID NO: 2 or SEQ ID NO: 4; and

(h) a nucleotide sequence complementary to any of (a)-(e).

The invention also provides for an isolated polypeptide comprising the amino acid sequence selected from the group consisting of:

(a) the mature amino acid sequence set forth in SEQ ID NO: 2 or SEQ ID NO: 4 comprising a mature amino terminus at residue 1, and optionally further comprising an amino-terminal methionine;

(b) an amino acid sequence for an ortholog of SEQ ID NO: 2 or SEQ ID NO: 4, wherein the polypeptide has an activity of the polypeptide set forth in SEQ ID NO: 2 or SEQ ID NO: 4;

(c) an amino acid sequence that is at least about 70, 75, 80, 85, 90, 95, 96, 97, 98, or 99 percent identical to the amino acid sequence of SEQ ID NO: 2 or SEQ ID NO: 4, wherein the polypeptide has an activity of the polypeptide set forth in SEQ ID NO: 2 or SEQ ID NO: 4 as determined using a computer program selected from the group consisting of GAP, BLASTP, BLASTN, FASTA, BLASTA, BLASTX, BestFit, and the Smith-Waterman algorithm;

(d) a fragment of the amino acid sequence set forth in SEQ ID NO: 2 or SEQ ID NO: 4 comprising at least about 25 amino acid residues, wherein the polypeptide has an activity of the polypeptide set forth in SEQ ID NO: 2 or SEQ ID NO: 4;

(e) an amino acid sequence for an allelic variant or splice variant of either the amino acid sequence set forth in SEQ ID NO: 2 or SEQ ID NO: 4, or at least one of (a)-(c) wherein the polypeptide has an activity of the polypeptide set forth in SEQ ID NO: 2 or SEQ ID NO: 4.

The invention further provides for an isolated polypeptide comprising the amino acid sequence selected from the group consisting of:

(a) the amino acid sequence set forth in SEQ ID NO: 2 or SEQ ID NO: 4 with at least one conservative amino acid substitution, wherein the polypeptide has an activity of the polypeptide set forth in SEQ ID NO: 2 or SEQ ID NO: 4;

(b) the amino acid sequence set forth in SEQ ID NO: 2 or SEQ ID NO: 4 with at least one amino acid insertion, wherein the polypeptide has an activity of the polypeptide set forth in SEQ ID NO: 2 or SEQ ID NO: 4;

(c) the amino acid sequence set forth in SEQ ID NO: 2 or SEQ ID NO: 4 with at least one amino acid deletion, wherein the polypeptide has an activity of the polypeptide set forth in SEQ ID NO: 2 or SEQ ID NO: 4;

(d) the amino acid sequence set forth in SEQ ID NO: 2 or SEQ ID NO: 4 which has a C- and/or N-terminal truncation, wherein the polypeptide has an activity of the polypeptide set forth in SEQ ID NO: 2 or SEQ ID NO: 4; and

(e) the amino acid sequence set forth in SEQ ID NO: 2 or SEQ ID NO: 4, with at least one modification selected from the group consisting of amino acid substitutions, amino acid insertions, amino acid deletions, C-terminal truncation, and N-terminal truncation, wherein the polypeptide has an activity of the polypeptide set forth in SEQ ID NO: 2 or SEQ ID NO: 4.

Also provided are fusion polypeptides comprising the polypeptide sequences of (a)-(e) above of the preceding paragraphs.

The present invention also provides for an expression vector comprising the isolated nucleic acid molecules set forth herein, recombinant host cells comprising recombinant nucleic acid molecules set forth herein, and a method of producing a TNFr/OPG-like polypeptide comprising culturing the host cells and optionally isolating the polypeptide so produced. These expression vectors include baculovirus expression vectors which utilize insect cells for expression.

A transgenic non-human animal comprising a nucleic acid molecule encoding a TNFr/OPG-like polypeptide is also encompassed by the invention. The TNFr/OPG-like nucleic acid molecules are introduced into the animal in a manner that allows expression and increased levels of the TNFr/OPG-like polypeptide, which may include increased circulating levels. The transgenic non-human animal is preferably a mammal. Also provided is a transgenic non-human animal comprising a disruption in the nucleic acid molecule encoding a TNFr/OPG-like polypeptide, which will knock-out or significantly decrease expression of the TNFr/OPG-like polypeptide.

Also provided are derivatives of the TNFr/OPG-like polypeptides of the present invention.

Analogs of TNFr/OPG-like are provided for in the present invention which result from conservative and non-conservative amino acids substitutions of the TNFr/OPG-like polypeptide of SEQ ID NO: 2 or SEQ ID NO: 4. Such analogs include a TNFr/OPG-like polypeptide wherein the amino acid at position 42 of SEQ ID NO: 2 is selected from the group consisting of proline and glycine, the amino acid at position 51 of SEQ ID NO: 2 is selected from the group consisting of serine, threoinine, asparagine, glutamine, the amino acid at position 56 of SEQ ID NO: 2 is selected from the group consisting of phenylalanine, tryptophan, and tyrosine, the amino acid at position 68 of SEQ ID NO: 2 is selected from the group consisting of histadine, lysine, and arginine, the amino acid at position 71 of SEQ ID NO: 2 is selected from the group consisting of serine, cysteine, theonine, asparagine, glutamine, the amino acid at position 84 of SEQ ID NO: 2 is selected from the group consisting of alanine, methionine, valine, leucine, isoleucine and norleucine or the amino acid at position 87 of SEQ ID NO: 2 is selected from the group consisting of aspartic acid or glutamic acid.

Additionally provided are selective binding agents such as antibodies and peptides capable of specifically binding the TNFr/OPG-like polypeptides of the invention. Such antibodies, polypeptides, peptides and small molecules may be agonistic or antagonistic.

Pharmaceutical compositions comprising the nucleotides, polypeptides, or selective binding agents of the present invention and one or more pharmaceutically acceptable formulation agents are also encompassed by the invention. The pharmaceutical compositions are used to provide therapeutically effective amounts of the nucleotides or polypeptides of the present invention. The invention is also directed to methods of using the polypeptides, nucleic acid molecules, and selective binding agents. The invention also provides for devices to administer a TNFr/OPG-like polypeptide encapsulated in a membrane.

The TNFr/OPG-like polypeptides and nucleic acid molecules of the present invention may be used to treat, prevent, ameliorate, diagnose and/or detect diseases and disorders, including those recited herein. Expression analysis in biological, cellular or tissue samples suggests that TNFr/OPG-like polypeptide may play a role in the diagnosis and/or treatment of the pathological conditions described herein. This expression can de detected with a diagnostic agent such as a TNFr/OPG-like polynucleotide.

The invention encompasses diagnosing a pathological condition or a susceptibility to a pathological condition in a subject caused by or resulting from abnormal levels of TNFr/OPG-like polypeptide comprising determining the presence or amount of expression of the TNFr/OPG-like polypeptide in a sample; and comparing the level of said polypeptide in a biological, tissue or cellular sample from either normal subjects or the subject at an earlier time, wherein susceptibility to a pathological condition is based on the presence or amount of expression of the polypeptide.

The present invention also provides a method of assaying test molecules to identify a test molecule which binds to a TNFr/OPG-like polypeptide. The method comprises contacting a TNFr/OPG-like polypeptide with a test molecule and to determine the extent of binding of the test molecule to the polypeptide. The method further comprises determining whether such test molecules are agonists or antagonists of a TNFr/OPG-like polypeptide. The present invention further provides a method of testing the impact of molecules on the expression of TNFr/OPG-like polypeptide or on the activity of TNFr/OPG-like polypeptide.

The present invention provides for methods of identifying antagonists of TNFr/OPG-like biological activity comprising contacting a small molecule compound with TNFr/OPG polypeptides and measuring TNFr/OPG-like biological activity in the presence and absence of these small molecules. These small molecules can be a naturally occurring medicinal compound or derived from combinational chemical libraries. In addition, the present invention also encompasses methods which identify TNFr/OPG-like binding partners, such as cyclins. These methods utilize a yeast two-hybrid approach comprising a bait construct consisting of a TNFr/OPG-like polynucleotide fused to GAL4 DNA binding domain. The bait construct is used to screen a cDNA library, wherein the library consists of nucleotide sequences fused to a GAL4 activation domain. Library sequences encoding TNFr/OPG-like interacting proteins can be identified by the transcriptional activation of reporter genes under the control of GAL4. See Guarente, Trend Gen., 9: 342-346 (1993); Bartel & Field, Meth. Enz., 254: 241-63 (1995).

Methods of regulating expression and modulating (i.e., increasing or decreasing) levels of a TNFr/OPG-like polypeptide are also encompassed by the invention. One method comprises administering to an animal a nucleic acid molecule encoding a TNFr/OPG-like polypeptide. In another method, a nucleic acid molecule comprising elements that regulate or modulate the expression of a TNFr/OPG-like polypeptide may be administered. Examples of these methods include gene therapy, cell therapy, and anti-sense therapy as further described herein.

In another aspect of the present invention, the TNFr/OPG-like polypeptides may be used for identifying binding partners thereof (“TNFr/OPG-like polypeptide binding partners”). Yeast two-hybrid screens have been extensively used to identify and clone binding partners and receptors for proteins. (Chien et al., Proc. Natl. Acad. Sci. USA, 88:9578-9583, 1991) The isolation of a TNFr/OPG-like polypeptide binding partner(s) is useful for identifying or developing novel agonists and antagonists of the TNFr/OPG-like polypeptide activity.

Such agonists and antagonists include soluble TNFr/OPG cofactors, anti-TNFr/OPG selective binding agents (such as TNFr/OPG-like antibodies and derivatives thereof), small molecules, peptides or derivatives thereof capable of binding TNFr/OPG-like polypeptides, or an antisense oligonucleotides, any of which can be used for potentially treating one or more diseases or disorders, including those recited herein. These pathological conditions include, but are not limited to, osteoporosis, Paget's disease, osteomyelitis, hypercalcemia, osteopenia and osteonecrosis.

In certain embodiments, a TNFr/OPG-like polypeptide agonist or antagonist may be a protein, peptide, carbohydrate, lipid, or small molecular weight molecule which interacts with TNFr/OPG-like polypeptide to regulate its activity.

### BRIEF DESCRIPTION OF THE FIGURES

FIG. 1 is SEQ ID NO: 1, and sets forth the cDNA sequence of the human TNFr/OPG-like nucleic acid molecule.

FIG. 2 is SEQ ID NO: 3, and sets forth the cDNA sequence of the mouse TNFr/OPG-like nucleic acid molecule.

FIG. 3 is SEQ ID NO: 2, and sets forth the amino acid sequence of the human TNFr/OPG-like polypeptide. In this figure, the predicted leader sequence is set forth in boldface, and the predicted transmembrane region is underlined.

FIG. 4 is SEQ ID NO: 4, and sets forth the amino acid sequence of the mouse TNFr/OPG-like polypeptide. In this figure, the predicted leader sequence is set forth in boldface, and the predicted transmembrane region is underlined.

FIG. 5 sets forth an overlap of the cDNA (CDR of SEQ ID NO: 1) and predicted amino acid sequence of the human TNFr/OPG-like polypeptide (SEQ ID NO: 2).

FIG. 6 sets forth an overlap of the cDNA (CDR of SEQ ID NO: 3) and predicted amino acid sequence of the mouse TNFr/OPG-like polypeptide (SEQ ID NO: 4).

FIG. 7 and sets forth the 543 nucleotide DNA fragment obtained through homology-based BLAST searches of a human genomic database (SEQ ID. NO: 5). In this figure, the predicted splicing donor (GTa) and acceptor (cAG) sequences are underlined. The predicted aminpo acid sequence (SEQ ID NO: 6) of this fragment is also shown.

FIG. 8 sets forth an amino acid sequence comparison of human osteoprotogerin (OPG; SEQ ID NO: 8) with TNFr/OPG-like polypeptide (SEQ ID NO: 7). SEQ ID NO: 7 represents amino acids 41 to 96 of SEQ ID NO: 2.

FIG. 9 shows the Western blot analysis of the TNFr/OPG-like Fc fusion protein that determined the TNFr/OPG-like fusion protein is cleaved by furin (left panel). The right panel displays the immunoprecipitation of full length TNFr/OPG-like recptor containing a N-terminal Flag tag from the conditioned media of TNFr/OPG-like-Fc fusion protein overexpressing 293-T cells.

FIG. 10 shows the flow cytometry studies performed on 20 cells lines. This analysis determined TNFr/OPG-like receptor extracellular domain binds to to Wehi-3 cells.

FIG. 11 shows Northern blot analysis detecting expression of TNFr/OPG-like mRNA in various tissues.

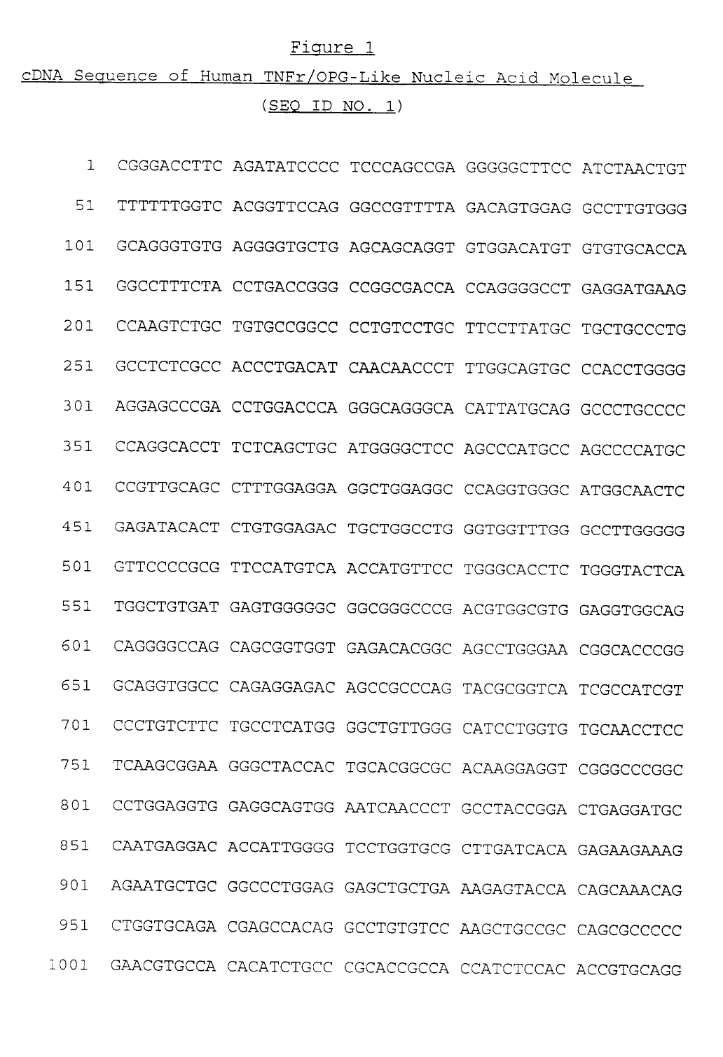

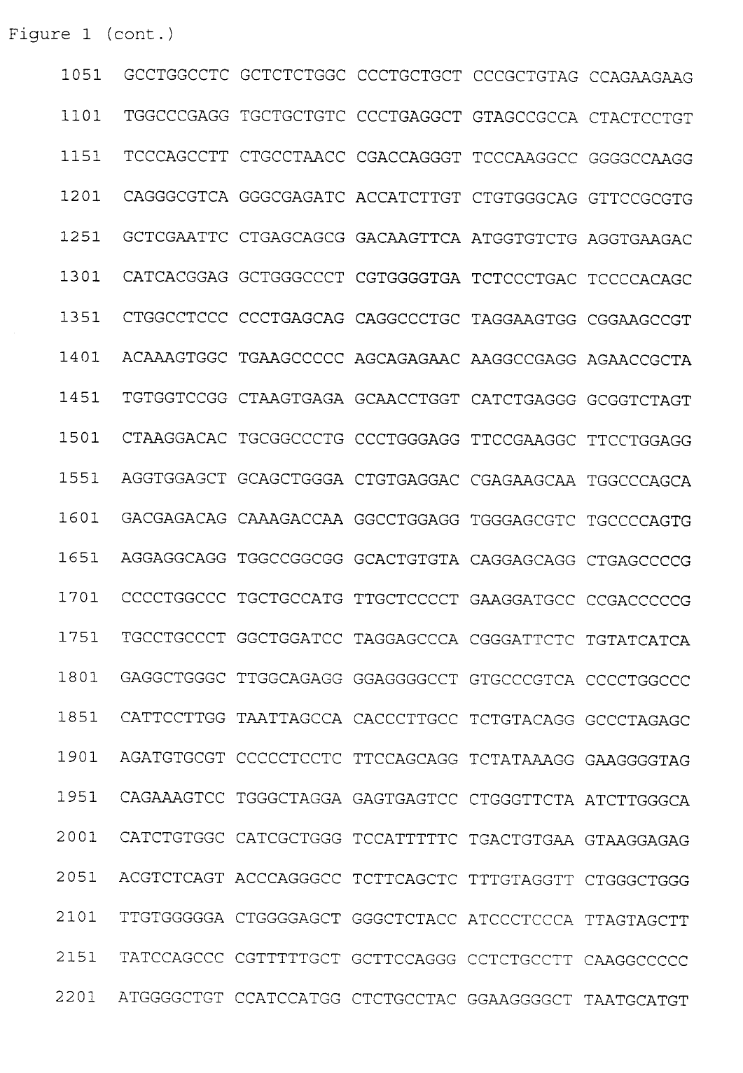

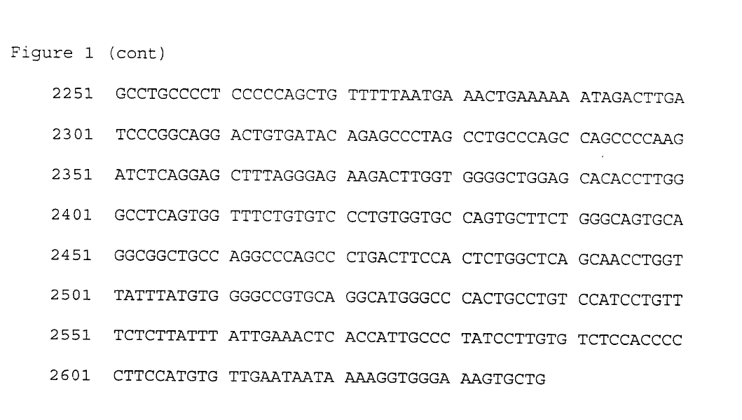

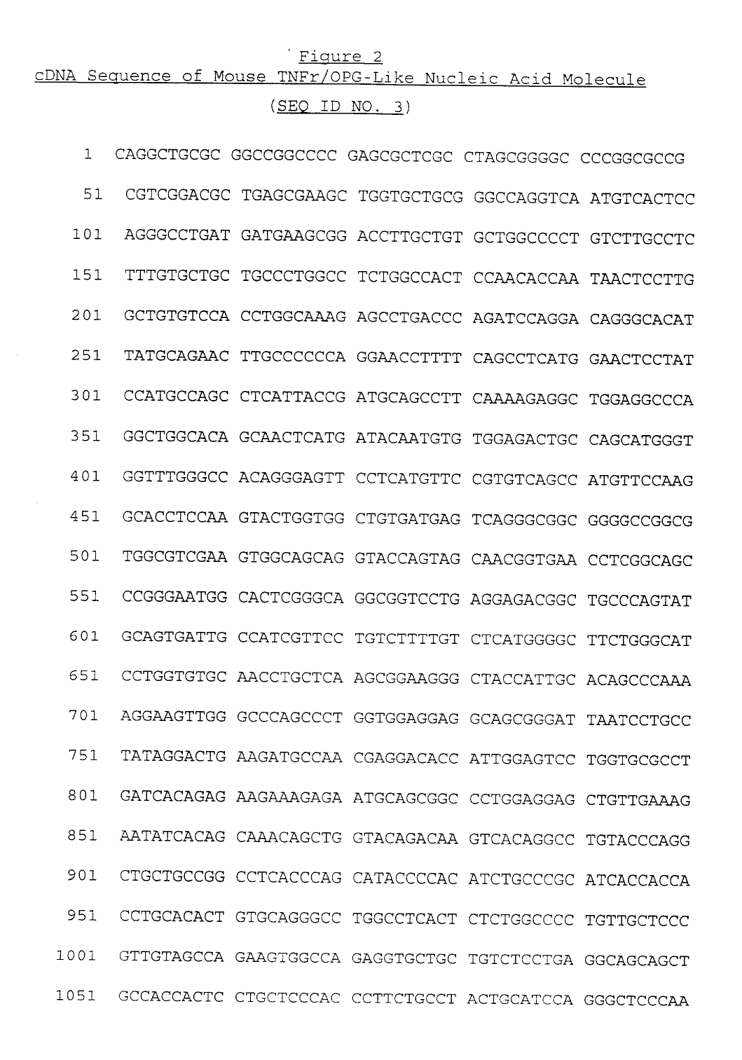

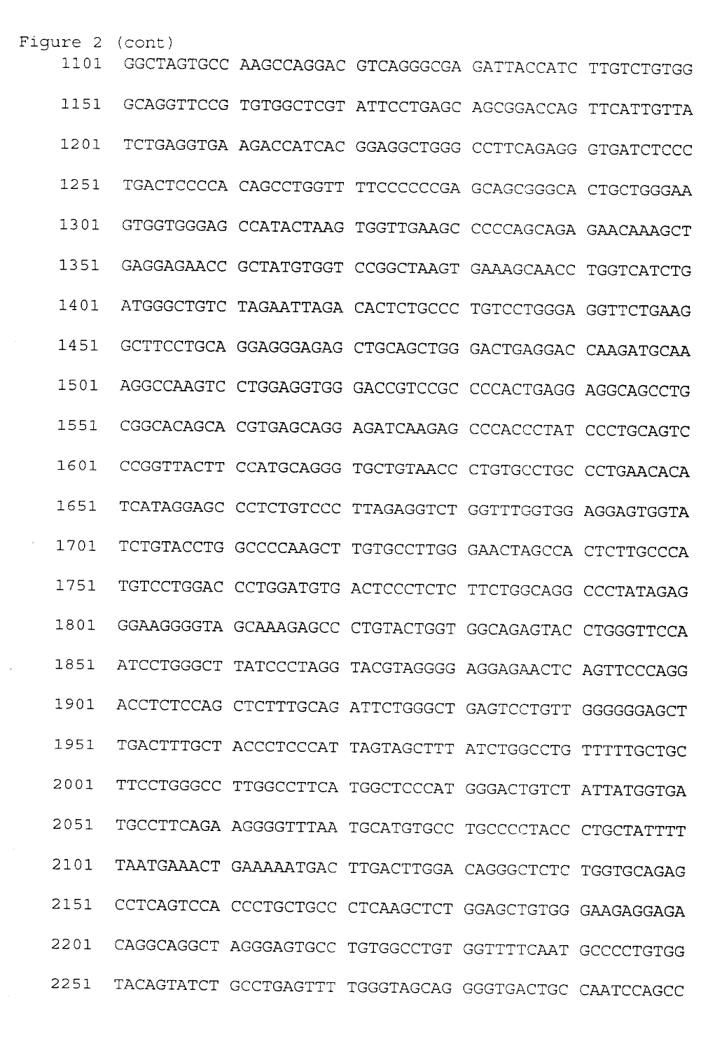

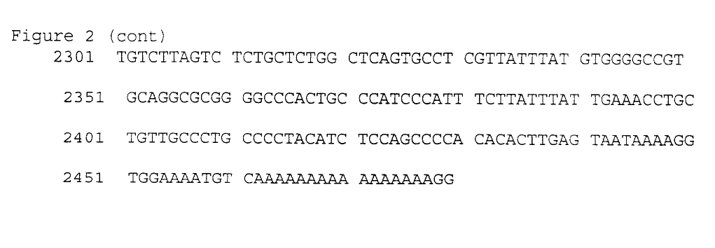

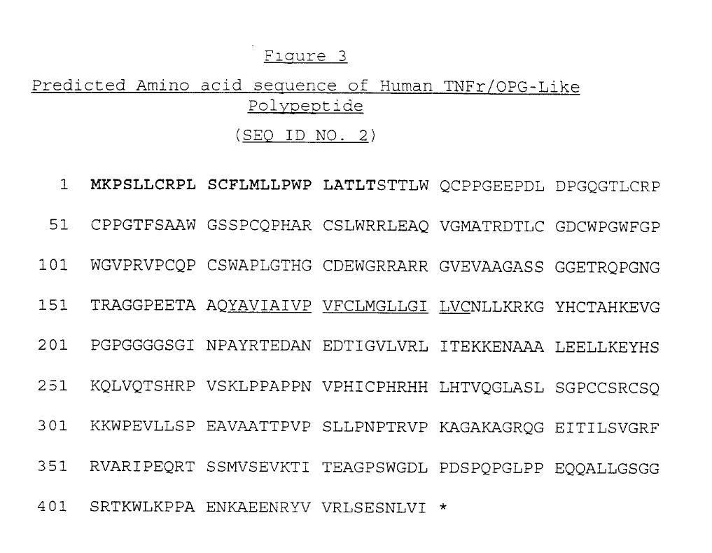

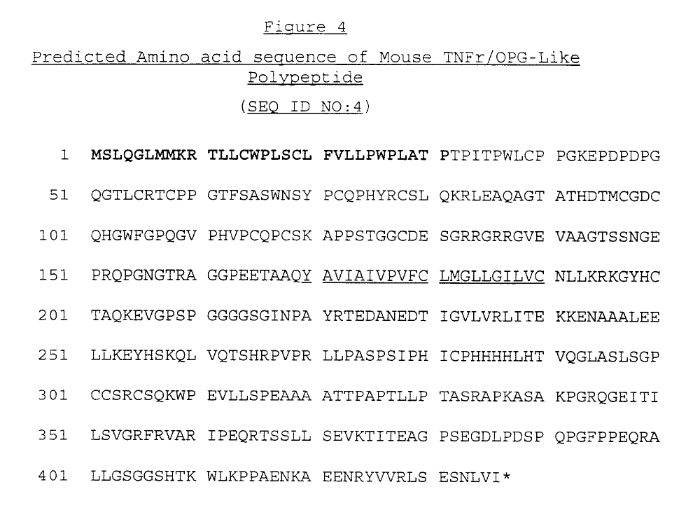

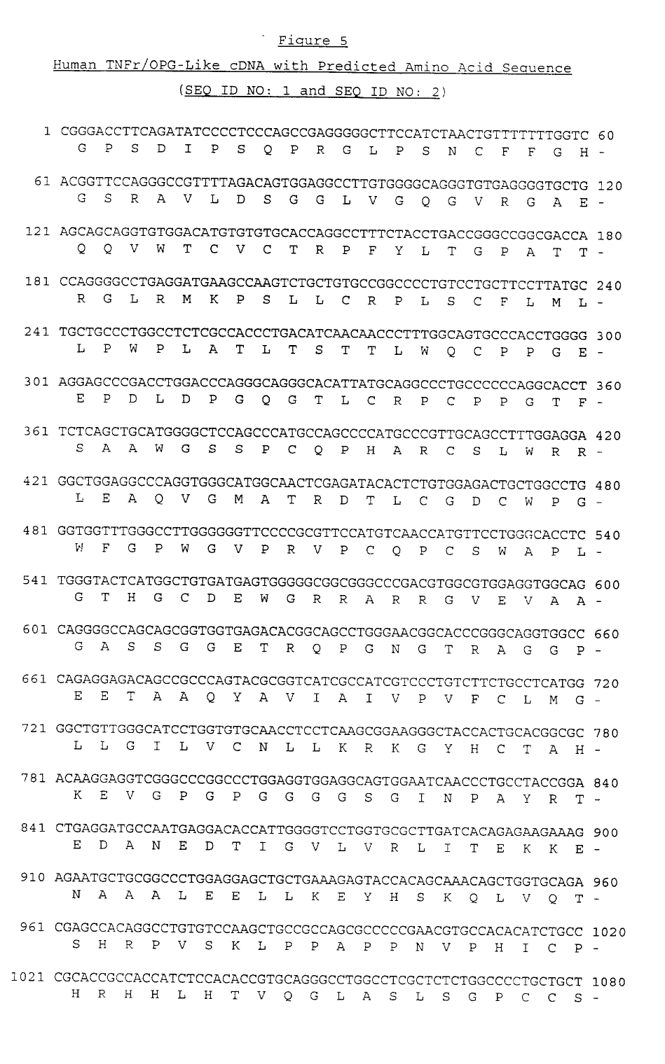

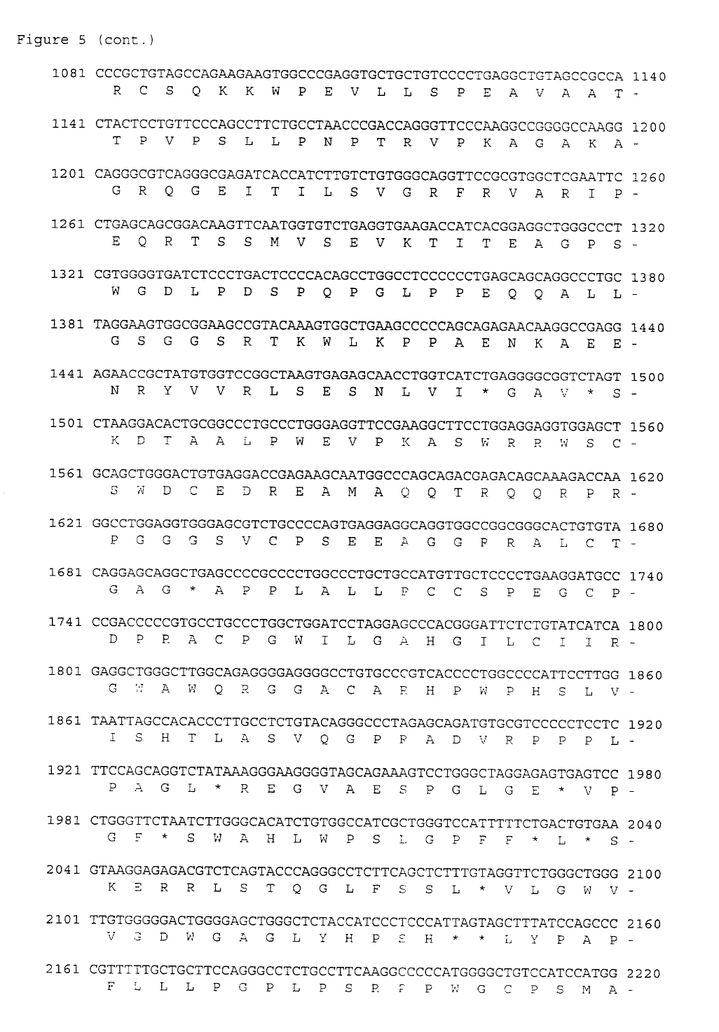

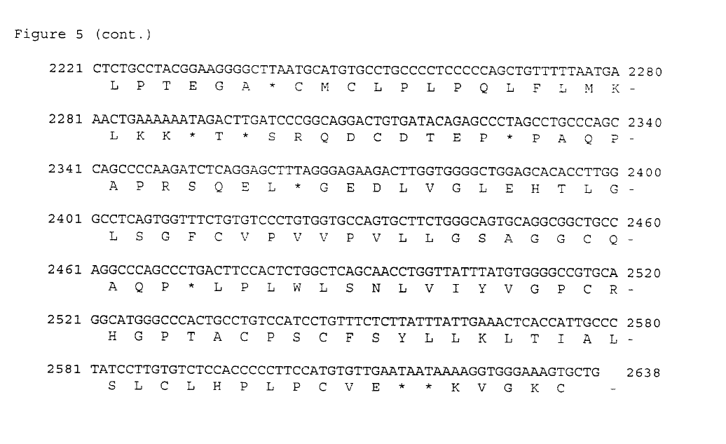

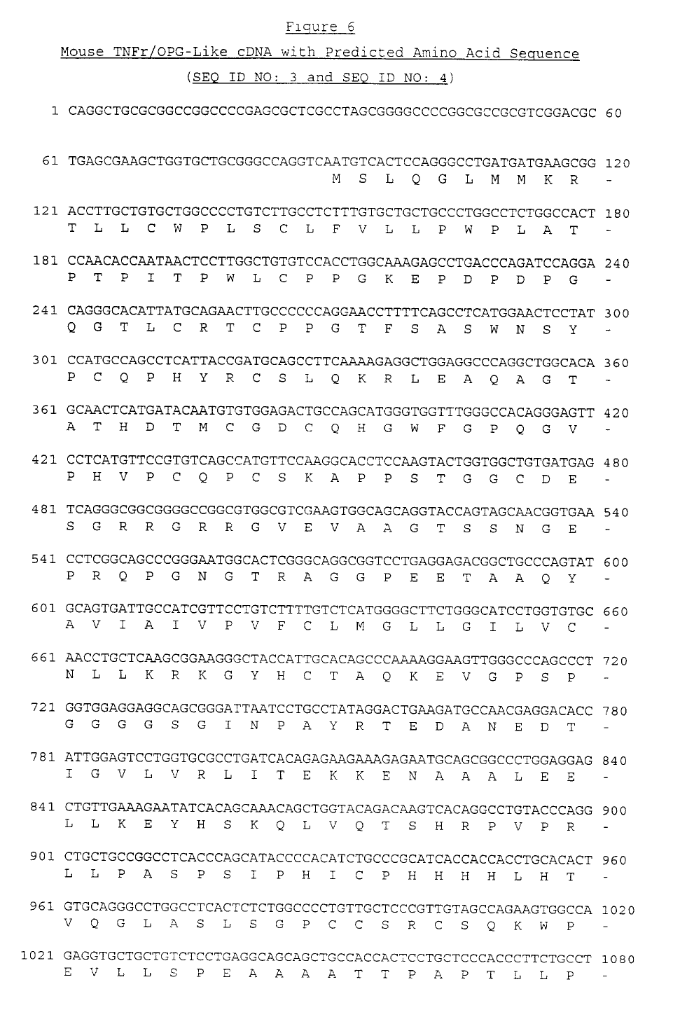

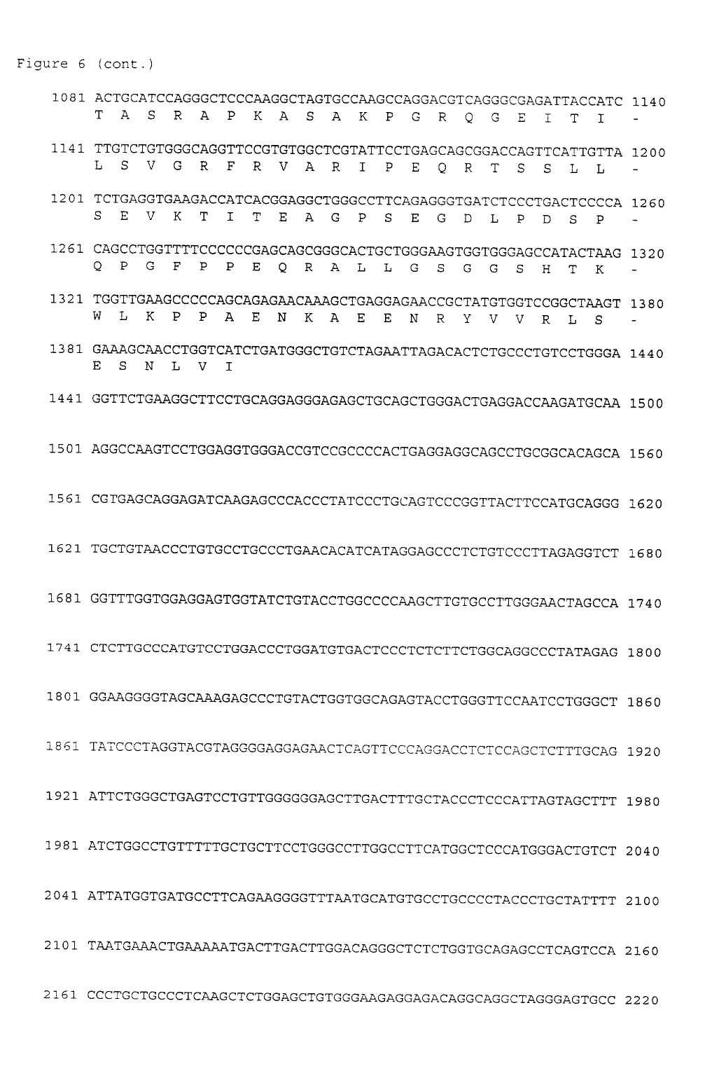

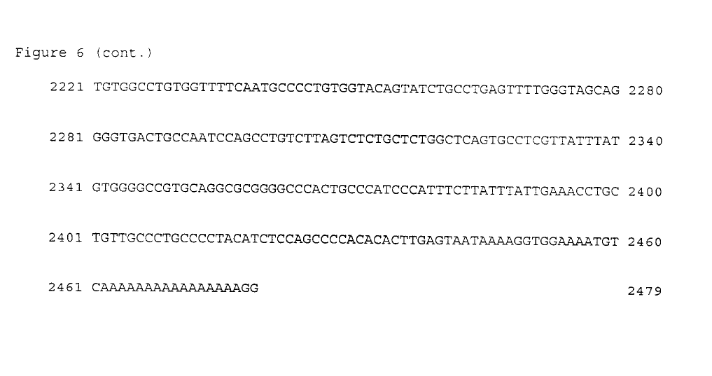

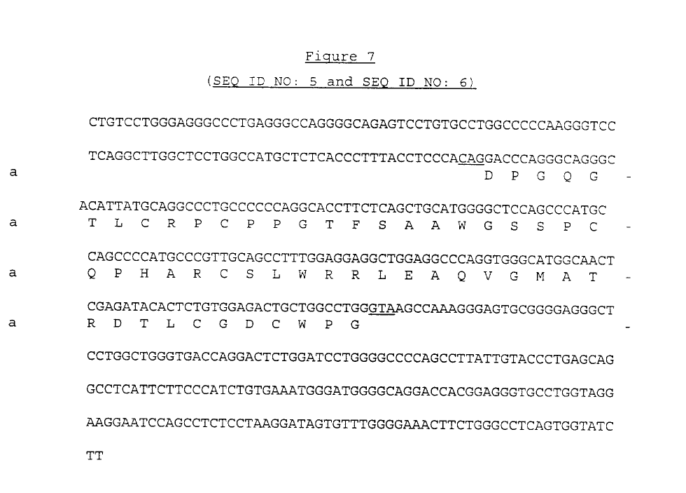

### DETAILED DESCRIPTION OF THE INVENTION

The section headings used herein are for organizational purposes only and are not to be construed as limiting the subject matter described therein. All references cited in this application are expressly incorporated by reference herein.

Definitions

The term “TNFr/OPG-like nucleic acid molecule” or “polynucleotide” refers to a nucleic acid molecule comprising or consisiting of a nucleotide sequence as set forth in either SEQ ID NOS: 1 or 3, a nucleotide sequence encoding the polypeptide as set forth in either SEQ ID NO: 2 or SEQ ID NO: 4, a nucleotide sequence of a DNA insert in ATCC deposit no. PTA-1758 and nucleic acid molecules as defined herein. Related nucleic acid molecules include a nucleotide sequence that is at least about 70 percent identical to the nucleotide sequence as shown in either SEQ ID NOS: 1 or 3, or comprise or consist essentially of a nucleotide sequence encoding a polypeptide that is at least about 70 percent identical to the polypeptide as set forth in either SEQ ID NO: 2 or SEQ ID NO 4. In preferred embodiments, the nucleotide sequences are about 75 percent, or about 80 percent, or about 85 percent, or about 90 percent, or about 95, 96, 97, 98, or 99 percent identical to the nucleotide sequence as shown in either SEQ ID NOS: 1 or 3, or the nucleotide sequences encode a polypeptide that is about 75 percent, or about 80 percent, or about 85 percent, or about 90 percent, or about 95, 96, 97, 98, or 99 percent identical to the polypeptide sequence as set forth in either SEQ ID NO: 2 or SEQ ID NO: 4.

Related nucleic acid molecules also include fragments of the TNFr/OPG-like nucleic acid molecules which fragments contain at least about 10 contiguous nucleotides, or about 15, or about 20, or about 25, or about 50, or about 75, or about 100, or greater than about 100 contiguous nucleotides of a TNFr/OPG-like nucleic acid molecule of either SEQ ID NOS: 1 or 3. Related nucleic acid molecules also include fragments of the above TNFr/OPG-like nucleic acid molecules which encode a polypeptide of at least about 25 amino acid residues, or about 50, or about 75, or about 100, or greater than about 100 amino acid residues of the TNFr/OPG-like polypeptide of either SEQ ID NO: 2 or SEQ ID NO: 4. Related nucleic acid molecules also include a nucleotide sequence encoding a polypeptide comprising or consisting essentially of a substitution, modification, addition and/or a deletion of one or more amino acid residues compared to the polypeptide set forth in either SEQ ID NO: 2 or SEQ ID NO: 4. In addition, related TNFr/OPG-like nucleic acid molecules include those molecules which comprise nucleotide sequences which hybridize under moderately or highly stringent conditions as defined herein with the fully complementary sequence of any of the TNFr/OPG-like nucleic acid molecules of either SEQ ID NOS: 1 or 3. In preferred embodiments, the related nucleic acid molecules comprise sequences which hybridize under moderately or highly stringent conditions with a complement of a molecule having a sequence as shown in either SEQ ID NOS: 1 or 3, or of a molecule encoding a polypeptide, which polypeptide comprises the sequence as shown in either SEQ ID NO: 2 or SEQ ID NO: 4 or of a nucleic acid fragment as defined herein, or of a nucleic acid fragment encoding a polypeptide as defined herein. It is also understood that related nucleic acid molecules include allelic or splice variants of a TNFr/OPG-like nucleic acid molecule of either SEQ ID NOS: 1 or 3, and include sequences which are complementary to any of the above nucleotide sequences. The related encoded polypeptides possess at least one activity of the polypeptide depicted in either SEQ ID NO: 2 or SEQ ID NO: 4.

The term “isolated nucleic acid molecule” refers to a nucleic acid molecule of the invention that (1) has been separated from at least about 50 percent of proteins, lipids, carbohydrates or other materials with which it is naturally found when total DNA is isolated from the source cells, (2) is not linked to all or a portion of a polynucleotide to which the “isolated nucleic acid molecule” is linked in nature, (3) is operably linked to a polynucleotide which it is not linked to in nature, or (4) does not occur in nature as part of a larger polynucleotide sequence. Preferably, the isolated nucleic acid molecule of the present invention is substantially free from any other contaminating nucleic acid molecule(s) or other contaminants that are found in its natural environment that would interfere with its use in polypeptide production or its therapeutic, diagnostic, prophylactic or research use.

A “nucleic acid sequence” or “nucleic acid molecule” refers to as used herein refers to a DNA or RNA sequence. The term encompasses molecules formed from any of the known base analogs of DNA and RNA such as, but not limited to 4-acetylcytosine, 8-hydroxy-N6-methyladenosine, aziridinyl-cytosine, pseudoisocytosine, 5-(carboxyhydroxylmethyl) uracil, 5-fluorouracil, 5-bromouracil, 5-carboxymethylaminomethyl-2-thiouracil, 5-carboxy-methylaminomethyluracil, dihydrouracil, inosine, N6-iso-pentenyladenine, 1-methyladenine, 1-methylpseudouracil, 1-methylguanine, 1-methylinosine, 2,2-dimethyl-guanine, 2-methyladenine, 2-methylguanine, 3-methylcytosine, 5-methylcytosine, N6-methyladenine, 7-methylguanine, 5-methylaminome-thyluracil, 5-methoxyaminomethyl-2-thiouracil, beta-D-mannosylqueosine, 5′-methoxycarbonyl-methyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

The term “operably linked” is used herein to refer to an arrangement of flanking sequences wherein the flanking sequences so described are configured or assembled so as to perform their usual function. Thus, a flanking sequence operably linked to a coding sequence may be capable of effecting the replication, transcription and/or translation of the coding sequence. For example, a coding sequence is operably linked to a promoter when the promoter is capable of directing transcription of that coding sequence. A flanking sequence need not be contiguous with the coding sequence, so long as it functions correctly. Thus, for example, intervening untranslated yet transcribed sequences can be present between a promoter sequence and the coding sequence and the promoter sequence can still be considered “operably linked” to the coding sequence.

The term “naturally occurring” or “native” when used in connection with biological materials such as nucleic acid molecules, polypeptides, host cells, and the like, refers to materials which are found in nature and are not manipulated by man. Similarly, “non-naturally occurring” or “non-native” as used herein refers to a material that is not found in nature or that has been structurally modified or synthesized by man.

The term “allelic variant” refers to one of several possible naturally occurring alternate forms of a gene occupying a given locus on a chromosome of an organism or a population of organisms.

The term “TNFr/OPG-like splice variant” refers to a nucleic acid molecule, usually RNA, which is generated by alternative processing of intron sequences in an RNA transcript of TNFr/OPG-like polypeptide amino acid sequences as set forth in SEQ ID NOS: 2 or 4.

The term “expression vector” refers to a vector which is suitable for use in a host cell and contains nucleic acid sequences which direct and/or control the expression of inserted heterologous nucleic acid sequences. Expression includes, but is not limited to, processes such as transcription, translation, and RNA splicing, if introns are present.

The term “host cell” is used to refer to a cell which has been transformed, or is capable of being transformed with a nucleic acid sequence and then of expressing a selected gene of interest. The term includes the progeny of the parent cell, whether or not the progeny is identical in morphology or in genetic make-up to the original parent, so long as the selected gene is present.

The term “vector” is used to refer to any molecule (e.g., nucleic acid, plasmid, or virus) used to transfer coding information to a host cell.

The term “host cell” . . . .

The term “transformation” as used herein refers to a change in a cell's genetic characteristics, and a cell has been transformed when it has been modified to contain a new DNA. For example, a cell is transformed where it is genetically modified from its native state. Following transfection or transduction, the transforming DNA may recombine with that of the cell by physically integrating into a chromosome of the cell, may be maintained transiently as an episomal element without being replicated, or may replicate independently as a plasmid. A cell is considered to have been stably transformed when the DNA is replicated with the division of the cell.

The term “transfection” is used to refer to the uptake of foreign or exogenous DNA by a cell, and a cell has been “transfected” when the exogenous DNA has been introduced inside the cell membrane. A number of transfection techniques are well known in the art and are disclosed herein. See, for example, Graham et al., Virology, 52:456 (1973); Sambrook et al., Molecular Cloning, a laboratory Manual, Cold Spring Harbor Laboratories (New York, 1989); Davis et al., Basic Methods in Molecular Biology, Elsevier, 1986; and Chu et al., Gene, 13:197 (1981). Such techniques can be used to introduce one or more exogenous DNA moieties into suitable host cells.

The term “transduction” is used to refer to the transfer of genes from one bacterium to another, usually by a phage. “Transduction” also refers to the acquisition and transfer of eukaryotic cellular sequences by retroviruses.

The term “host cell” is used to refer to a cell which has been transformed, or is capable of being transformed, by a vector bearing a selected gene of interest which is then expressed by the cell. The term includes the progeny of the parent cell, whether or not the progeny is identical in morphology or in genetic make-up to the original parent, so long as the selected gene is present.

The term “highly stringent conditions” refers to those conditions that are designed to permit hybridization of DNA strands whose sequences are highly complementary, and to exclude hybridization of significantly mismatched DNAs. Hybridization stringency is principally determined by temperature, ionic strength, and the concentration of denaturing agents such as formamide. Examples of “highly stringent conditions” for hybridization and washing are 0.015M sodium chloride, 0.0015M sodium citrate at 65-68° C. or 0.015M sodium chloride, 0.0015M sodium citrate, and 50% formamide at 42° C. See Sambrook, Fritsch & Maniatis, Molecular Cloning: A Laboratory Manual, 2^{nd} Ed., Cold Spring Harbor Laboratory, (Cold Spring Harbor, N.Y. 1989); Anderson et al., Nucleic Acid Hybridisation: a practical approach, Ch. 4, IRL Press Limited (Oxford, England).

More stringent conditions (such as higher temperature, lower ionic strength, higher formamide, or other denaturing agent) may also be used, however, the rate of hybridization will be affected. Other agents may be included in the hybridization and washing buffers for the purpose of reducing non-specific and/or background hybridization. Examples are 0.1% bovine serum albumin, 0.1% polyvinyl-pyrrolidone, 0.1% sodium pyrophosphate, 0.1% sodium dodecylsulfate, NaDodSO₄ or(SDS), ficoll, Denhardt's solution, sonicated salmon sperm DNA (or other non-complementary DNA), and dextran sulfate, although other suitable agents can also be used. The concentration and types of these additives can be changed without substantially affecting the stringency of the hybridization conditions. Hybridization experiments are usually carried out at pH 6.8-7.4, however, at typical ionic strength conditions, the rate of hybridization is nearly independent of pH. See Anderson et al., Nucleic Acid Hybridisation: a Practical Approach, Ch. 4, IRL Press Limited (Oxford, England).

Factors affecting the stability of a DNA duplex include base composition, length, and degree of base pair mismatch. Hybridization conditions can be adjusted by one skilled in the art in order to accommodate these variables and allow DNAs of different sequence relatedness to form hybrids. The melting temperature of a perfectly matched DNA duplex can be estimated by the following equation:

Tₘ (° C.)=81.5+16.6(log[Na+])+0.41(%G+C)−600/N−0.72(%formamide)

where N is the length of the duplex formed, [Na+] is the molar concentration of the sodium ion in the hybridization or washing solution, %G+C is the percentage of (guanine+cytosine) bases in the hybrid. For imperfectly matched hybrids, the melting temperature is reduced by approximately 1° C. for each 1% mismatch.

The term “moderately stringent conditions” refers to conditions under which a DNA duplex with a greater degree of base pair mismatching than could occur under “highly stringent conditions” is able to form. Examples of typical “moderately stringent conditions” are 0.015M sodium chloride, 0.0015M sodium citrate at 50-65° C. or 0.015M sodium chloride, 0.0015M sodium citrate, and 20% formamide at 37-50° C. By way of example, a “moderately stringent” condition of 50° C. in 0.015 M sodium ion will allow about a 21% mismatch.

It will be appreciated by those skilled in the art that there is no absolute distinction between “highly” and “moderately” stringent conditions. For example, at 0.015M sodium ion (no formamide), the melting temperature of perfectly matched long DNA is about 71° C. With a wash at 65° C. (at the same ionic strength), this would allow for approximately a 6% mismatch. To capture more distantly related sequences, one skilled in the art can simply lower the temperature or raise the ionic strength.

A good estimate of the melting temperature in 1M NaCl* for oligonucleotide probes up to about 20 nt is given by:

Tm=2° C. per A-T base pair+4° C. per G-C base pair

*The sodium ion concentration in 6×salt sodium citrate (SSC) is 1M. See Suggs et al., Developmental Biology Using Purified Genes, p. 683, Brown and Fox (eds.) (1981).

High stringency washing conditions for oligonucleotides are usually at a temperature of 0-5° C. below the Tm of the oligonucleotide in 6×SSC, 0.1% SDS.

The term “TNFr/OPG-like polypeptide” refers to a polypeptide comprising the amino acid sequence of either SEQ ID NO: 2 or SEQ ID NO: 4, and related polypeptides having a natural sequence or mutated sequence. Related polypeptides include: allelic variants; splice variants; fragments; derivatives; substitution, deletion, and insertion variants; fusion polypeptides; and orthologs of the TNFr/OPG-like polypeptides of either SEQ ID NO: 2 or SEQ ID NO: 4, and which possess at least one activity of the polypeptide depicted in either SEQ ID NO: 2 or SEQ ID NO: 4. TNFr/OPG-like polypeptides may be mature polypeptides, as defined herein, and may or may not have an amino terminal methionine residue, depending on the method by which they are prepared.

The term “isolated polypeptide” refers to a polypeptide of the present invention that (1) has been separated from at least about 50 percent of polynucleotides, lipids, carbohydrates or other materials with which it is naturally found when isolated from the source cell, (2) is not linked (by covalent or noncovalent interaction) to all or a portion of a polypeptide to which the “isolated polypeptide” is linked in pature, (3) is operably linked (by covalent or noncovalent interaction) to a polypeptide with which it is not linked in nature, or (4) does not occur in nature. Preferably, the isolated polypeptide is substantially free from any other contaminating polypeptides or other contaminants that are found in its natural environment that would interfere with its therapeutic, diagnostic, prophylactic or research use.

The term “TNFr/OPG-like polypeptide fragment” refers to a polypeptide that comprises less than the full length amino acid sequence of a TNFr/OPG-like polypeptide as set forth in either SEQ ID NO: 2 or SEQ ID NO: 4. Such TNFr/OPG-like fragments can be 6 amino acids or more in length, and may arise, for example, from a truncation at the amino terminus (with or without a leader sequence), a truncation at the carboxy terminus, and/or an internal deletion of one or more residues from the amino acid sequence. TNFr/OPG-like fragments may result from alternative RNA splicing or from in vivo protease activity. Membrane-bound forms of a TNFr/OPG-like polypeptide are also contemplated by the present invention. In preferred embodiments, truncations and/or deletions comprise about 10 amino acids, or about 20 amino acids, or about 50 amino acids, or about 75 amino acids, or about 100 amino acids, or more than about 100 amino acids. The polypeptide fragments so produced will comprise about 25 contiguous amino acids, or about 50 amino acids, or about 75 amino acids, or about 100 amino acids, or about 150 amino acids, or about 200 amino acids. Such TNFr/OPG-like polypeptide fragments may optionally comprise an amino terminal methionine residue. It will be appreciated that such fragments can also be used, for example, to generate antibodies to TNFr/OPG-like polypeptides.

The term “TNFr/OPG-like polypeptide variants” refers to TNFr/OPG-like polypeptides which contain one or more amino acid sequence substitutions, deletions, and/or additions as compared to the TNFr/OPG-like polypeptide amino acid sequence set forth in either SEQ ID NO: 2 or SEQ ID NO: 4. Variants may be naturally occurring or artificially constructed. Such TNFr/OPG-like polypeptide variants may be prepared from the corresponding nucleic acid molecules encoding said variants, which have a DNA sequence that varies accordingly from the DNA sequences for wild type TNFr/OPG-like polypeptides as set forth in either SEQ ID NOS: 1 or 3. In preferred embodiments, the variants have form 1 to 3, or 1 to 5, or 1 to 10, or 1 to 15, or 1 to 20, or 1 to 25, or 1 to 50, or 1 to 75, or 1 to 100, or more than 100 amino acid substations, insertions, additions and/or deletions, wherein tehh substitutions may be conservative, or non-conservative, or any combination thereof.

One skilled in the art will be able to determine suitable variants of the native TNFr/OPG-like polypeptide using well known techniques. For example, one may predict suitable areas of the molecule that may be changed without destroying biological activity. Also, one skilled in the art will realize that even areas that may be important for biological activity or for structure may be subject to conservative amino acid substitutions without destroying the biological activity or without adversely affecting the polypeptide structure.

For example, when similar polypeptides with similar activities from the same species or from other species are known, one skilled in the art may compare the amino acid sequence of a TNFr/OPG-like polypeptide to such similar polypeptides. With such a comparison, one can identify residues and portions of the molecules that are conserved among similar polypeptides. It will be appreciated that changes in areas of a TNFr/OPG-like polypeptide that are not conserved relative to such similar polypeptides would be less likely to adversely affect the biological activity and/or structure of the TNFr/OPG-like like polypeptide. One skilled in the art would also know that, even in relatively conserved regions, one may substitute chemically similar amino acids for the naturally occurring residues while retaining activity (conservative amino acid residue substitutions). Therefore, even areas that may be important for biological activity or for structure may be subject to conservative amino acid substitutions without destroying the biological activity or without adversely affecting the polypeptide structure.

For predicting suitable areas of the molecule that may be changed without destroying activity, one skilled in the art may target areas not believed to be important for activity. For example, when similar polypeptides with similar activities from the same species or from other species are known, one skilled in the art may compare the amino acid sequence o f TNFr/OPG-like polypeptide to such similar polypeptides. After making such a comparison, one skilled in the art can determine residues and portions of the molecules that are conserved among similar polypeptides. One skilled in the art would know that changes in areas of the TNFr/OPG-like molecule that are not conserved would be less likely to adversely affect the biological activity and/or structure of a TNFr/OPG-like polypeptide. One skilled in the art would also know that, even in relatively conserved regions, one may substitute chemically similar amino acids for the naturally occurring residues while retaining activity (conservative amino acid residue substitutions).

Additionally, one skilled in the art can review structure-function studies identifying residues in similar polypeptides that are important for activity or structure. In view of such a comparison, one skilled in the art can predict the importance of amino acid residues in a TNFr/OPG-like polypeptide that correspond to amino acid residues that are important for activity or structure in similar polypeptides. One skilled in the art may opt for chemically similar amino acid substitutions for such predicted important amino acid residues of TNFr/OPG-like polypeptides.

If available, one skilled in the art can also analyze the three-dimensional structure and amino acid sequence in relation to that structure in similar polypeptides. In view of that information, one skilled in the art may predict the alignment of amino acid residues of TNFr/OPG-like polypeptide with respect to its three dimensional structure. One skilled in the art may choose not to make radical changes to amino acid residues predicted to be on the surface of the protein, since such residues may be involved in important interactions with other molecules.

TNFr/OPG-like polypeptide analogs of the invention can be determined by comparing the amino acid sequence of TNFr/OPG-like polypeptide with related family members. Exemplary TNFr/OPG-like polypeptide related family members include, but are not limited to, Osteoprotegerin (OPG), and the TNF receptor. This comparison can be accomplished by using a Pileup alignment (Wisconsin GCG Program Package) or an equivalent (overlapping) comparison with multiple family members within conserved and non-conserved regions.

As shown in FIG. 8, the predicted amino acid sequence of human TNFr/OPG-like polypeptide (SEQ ID NO: 7; which represent amino acid 41 to 96 of SEQ ID NO: 2) is aligned with human OPG (SEQ ID NO: 8). Other TNFr/OPG-like polypeptide analogs can be determined using these or other methods known to those of skill in the art. These overlapping sequences provide guidance for conservative and non-conservative amino acids substitutions resulting in additional TNFr/OPG-like analogs. It will be appreciated that these amino acid substitutions can consist of naturally occurring or non-naturally occurring amino acids. For example, as depicted in FIG. 8, alignment of the of related family members indicates potential TNFr/OPG analogs may have the Pro residue at position 42 of SEQ ID NO: 2 (position 37 on FIG. 8) substituted with a Gly residue, the Cys residue at position 51 of SEQ ID NO: 2 (position 46 on FIG. 8) substituted with a Ser, Thr, Asn or Glu residue and/or the Phe residue at position 56 of SEQ ID NO: 2 (position 51 on FIG. 8) substituted with a Trp, or Tyr residue. In addition, potential TNFr/OPG analogs may have the His residue at position 68 of SEQ ID NO: 2 (position 63 on FIG. 8) substituted with a Lys or Arg residue, the Ser residue at position 71 of SEQ ID NO: 2 (position 67 on FIG. 8) substituted with a Cys, Thr, Asn or Gln residue, the Ala residue at position 84 of SEQ ID NO: 2 (position 79 on FIG. 8) substituted with a Met, Val, Leu, Ile or norleucine residue, and/or the Asp residue at position 87 of SEQ ID NO: 2 (position 83 on FIG. 8) substituted with a Glu residue.

Moreover, one skilled in the art may generate test variants containing a single amino acid substitution at each amino acid residue. The variants could be screened using activity assays described herein. Such variants could be used to gather information about suitable variants. For example, if one discovered that a change to a particular amino acid residue resulted in destroyed, undesirably reduced, or unsuitable activity, variants with such a change would be avoided. In other words, based on information gathered from such routine experiments, one skilled in the art can readily determine the amino acids where further substitutions should be avoided either alone or in combination with other mutations.

In making such changes of an equivalent nature, the hydropathic index of amino acids may be considered. Each amino acid has been assigned a hydropathic index on the basis of their hydrophobicity and charge characteristics, these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is understood in the art. Kyte et al., J. Mol. Biol., 157:105-131 (1982). It is known that certain amino acids may be substituted for other amino acids having a similar hydropathic index or score and still retain a similar biological activity. In making changes based upon the hydropathic index, the substitution of amino acids whose hydropathic indices are within ±2 is preferred, those which are within ±1 are particularly preferred, and those within ±0.5 are even more particularly preferred.

It is also understood in the art that the substitution of like amino acids can be made effectively on the basis of hydrophilicity, particularly where the biologically functionally equivalent protein or peptide thereby created is intended for use in immunological embodiments, as in the present case.

U.S. Pat. No. 4,554,101 states that the greatest local average hydrophilicity of a protein, as governed by the hydrophilicity of its adjacent amino acids, correlates with its immunogenicity and antigenicity, i.e., with a biological property of the protein. As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4).

In making changes based upon similar hydrophilicity values, the substitution of amino acids whose hydrophilicity values are within ±2 is preferred, those which are within ±1 are particularly preferred, and those within ±0.5 are even more particularly preferred. U.S. Pat. No. 4,554,101 also teaches the identification and preparation of epitopes from primary amino acid sequences on the basis of hydrophilicity. Through the methods disclosed in U.S. Pat. No. 4,554,101 one of skill in the art is able to identify epitopes from within a given amino acid sequence. These regions are also referred to as “epitopic core regions”.

Desired amino acid substitutions (whether conservative or non-conservative) can be determined by those skilled in the art at the time such substitutions are desired. For example, amino acid substitutions can be used to identify important residues of the TNFr/OPG-like polypeptide, or to increase or decrease the affinity of the TNFr/OPG-like polypeptides described herein.

Numerous scientific publications have been devoted to the prediction of secondary structure, and to the identification of epitopes, from analyses of amino acid sequences. See Chou et al., Biochemistry, 13(2):222-245 (1974); Chou et al., Biochemistry, 113(2):211-222 (1974); Chou et al., Adv. Enzymol. Relat. Areas Mol. Biol., 47:45-148 (1978); Chou et al., Ann. Rev. Biochem., 47:251-276 and Chou et al., Biophys. J., 26:367-384 (1979). Moreover, computer programs are currently available to assist with predicting antigenic portions and epitopic core regions of proteins. Examples include those programs based upon the Jameson-Wolf analysis (Jameson et al., Comput. Appl. Biosci., 4(1):181-186 (1998) and Wolf et al., Comput. Appl. Biosci., 4(1):187-191 (1988), the program PepPlot® (Brutlag et al., CABS, 6:237-245 (1990), and Weinberger et al., Science, 228:740-742 (1985), and other new programs for protein tertiary structure prediction (Fetrow et al., Biotechnology, 11:479-483 (1993).

Moreover, computer programs are currently available to assist with predicting secondary structure. One method of predicting secondary structure is based upon homology modeling. For example, two polypeptides or proteins which have a sequence identity of greater than 30%, or similarity greater than 40% often have similar structural topologies. The recent growth of the protein structural data base ( PDB) has provided enhanced predictability of secondary structure, including the potential number of folds within a polypeptide's or protein's structure. See Holm et al., Nucl. Acid. Res., 27(1):244-247 (1999). It has been suggested (Brenner et al., Curr. Op. Struct. Biol., 7(3):369-376 (1997)) that there are a limited number of folds in a given polypeptide or protein and that once a critical number of structures have been resolved, structural prediction will gainbecome dramatically in accuracy.more accurate.

Additional methods of predicting secondary structure include “threading”“threading” (Jones, D., Curr. Opin. Struct. Biol., 7(3):377-87 (1997); Sippl et al., Structure, 4(1):15-9 (1996)), “profile analysis” 4(1):15-19 (1996)), “profile analysis” (Bowie et al., Science, 253:164-170 (1991); Gribskov et al., Meth. Enzym., 183:146-159 (1990); Gribskov et al., Proc. Nat. Acad. Sci., 84(13):4355-4358 (1987)), and “evolutionary linkage” (See Home, supra, “evolutionary linkage” (See Holm, supra (1999), and Brenner, supra).

In preferred embodiments, the variants have from 1 to 3, or from 1 to 5, or from 1 to 10, or from 1 to 15, or from 1 to 20, or from 1 to 25, or from 1 to 50, or from 1 to 75, or from 1 to 100, or more than 100 amino acid substitutions, insertions, additions and/or deletions, wherein the substitutions may be conservative, as described herein, or non-conservative, or any combination thereof. In addition, the variants can have additions of amino acid residues either at the carboxy terminus or at the amino terminus (with or without a leader sequence).

Preferred TNFr/OPG-like polypeptide variants include glycosylation variants wherein the number and/or type of glycosylation sites has been altered compared to native TNFr/OPG-like polypeptide. In one embodiment, TNFr/OPG-like polypeptide variants comprise a greater or a lesser number of N-linked glycosylation sites. An N-linked glycosylation site is characterized by the sequence: Asn—X—Ser or Thr, wherein the amino acid residue designated as X may be any amino acid residue except proline. The substitution(s) of amino acid residues to create this sequence provides a potential new site for the addition of an N-linked carbohydrate chain. Alternatively, substitutions which eliminate this sequence will remove an existing N-linked carbohydrate chain. Also provided is a rearrangement of N-linked carbohydrate chains wherein one or more N-linked glycosylation sites (typically those that are naturally occurring) are eliminated and one or more new N-linked sites are created. Additional preferred TNFr/OPG-like variants include cysteine variants, wherein one or more cysteine residues are deleted or substituted with another amino acid (e.g., serine). Cysteine variants are useful when TNFr/OPG-like polypeptides must be refolded into a biologically active conformation such as after the isolation of insoluble inclusion bodies. Cysteine variants generally have fewer cysteine residues than the native protein, and typically have an even number to minimize interactions resulting from unpaired cysteines.

The term “TNFr/OPG-like fusion polypeptide” refers to a fusion of TNFr/OPG-like polypeptide, fragment, and/or variant thereof, with a heterologous peptide or polypeptide. Heterologous peptides and polypeptides include, but are not limited to: an epitope to allow for the detection and/or isolation of a TNFr/OPG-like fusion polypeptide; a transmembrane receptor protein or a portion thereof, such as an extracellular domain, or a transmembrane and intracellular domain; a ligand or a portion thereof which binds to a transmembrane receptor protein; an enzyme or portion thereof which is catalytically active; a polypeptide or peptide which promotes oligomerization, such as a leucine zipper domain; a polypeptide or peptide which increases stability, such as an immunoglobulin constant region, and a polypeptide which has a therapeutic activity different from the TNFr/OPG-like polypeptide.

In addition, a TNFr/OPG-like polypeptide may be fused to itself or to a fragment, variant, or derivative thereof. Fusions can be made either at the amino terminus or at the carboxy terminus of a TNFr/OPG-like polypeptide. Fusions may be direct with no linker or adapter molecule or may be through a linker or adapter molecule, such as one or more amino acid residues up to about 20 amino acids residues, or up to about 50 amino acid residues. A linker or adapter molecule may also be designed with a cleavage site for a DNA restriction endonuclease or for a protease to allow for the separation of the fused moieties. It will be appreciated that once constructed, the fusion polypeptides can be derivatized according to the methods described herein.

In a further embodiment of the invention, a TNFr/OPG-like polypeptide, including a fragment, variant, and/or derivative, is fused to an Fc region of human IgG. Antibodies comprise two functionally independent narts, a variable domain known as “Fab”, which binds antigen, and a constant domain known as “Fc”, which links to such effector functions as complement activation and attack by phagocytic cells. An Fc has a long serum half-life, whereas an Fab is short-lived. Capon et al., Nature, 337: 525-31 (1989). When constructed together with a therapeutic protein, an Fc domain can provide longer half-life or incorporate such functions as Fc receptor binding, protein binding, complement fixation and perhaps even placental transfer. Id. Table I summarizes the use of certain Fc fusions known in the art, including materials and methods applicable to the production of fused TNFr/OPG-like polypeptides.

TABLE I
Fc fusion with therapeutic proteins

| | Fusion | Therapeutic | |
|---|---|---|---|
| Form of Fc | partner | implications | Reference |
| IgG1 | N-terminus | Hodgkin's | U.S. Pat. No. |
| | of CD30-L | disease; | 5,480,981 |
| | | anaplastic | |
| | | lymphoma; T-cell | |
| | | leukemia | |
| Murine | IL-10 | anti- | Zheng et al. |
| Fcγ2a | | inflammatory; | (1995) , J. |
| | | transplant | Immunol., 154: |
| | | rejection | 5590-600 |
| IgG1 | TNF | septic shock | Fisher et al. |
| | receptor | | (1996), N. Engl. |
| | | | J. Med., 334: |
| | | | 1697-1702; Van |
| | | | Zee et al., |
| | | | (1996) , J. |
| | | | Immunol., 156: |
| | | | 2221-30 |
| IgG, IgA, | TNF | inflammation, | U.S. Pat. No. |
| IgM, or | receptor | autoimmune | 5,808,029, issued |
| IgE | | disorders | Sep. 15, |
| (excluding | | | 1998 |
| the first | | | |
| domain) | | | |
| IgG1 | CD4 | AIDS | Capon et al. |
| | receptor | | (1989) , Nature |
| | | | 337: 525-31 |
| IgG1, | N-terminus | anti-cancer, | Harvill et al. |
| IgG3 | of IL-2 | antiviral | (1995) |
| | | | Immunotech., 1: |
| | | | 95-105 |
| IgG1 | C-terminus | osteoarthritis; | WO 97/23614, |
| | of OPG | bone density | published Jul. 3, |
| | | | 1997 |
| IgG1 | N-terminus | anti-obesity | PCT/US 97/23183, |
| | of leptin | | filed Dec. |
| | | | 11, 1997 |
| Human Ig | CTLA-4 | autoimmune | Linsley (1991), |
| Cγ1 | | disorders | J. Exp. Med., |
| | | | 174: 561-9 |

In one example, all or a portion of the human IgG hinge, CH2 and CH3 regions may be fused at either the N-terminus or C-terminus of the TNFr/OPG-like polypeptides using methods known to the skilled artisan. In another example, a portion of a hinge regions and CH2 and CH3 regions may be fused. The resulting TNFr/OPG-like Fc-fusion polypeptide may be purified by use of a Protein A affinity column. Peptides and proteins fused to an Fc region have been found to exhibit a substantially greater half-life in vivo than the unfused counterpart. Also, a fusion to an Fc region allows for dimerization/multimerization of the fusion polypeptide. The Fc region may be a naturally occurring Fc region, or may be altered to improve certain qualities, such as therapeutic qualities, circulation time, reduction of aggregation, etc.

The term “TNFr/OPG-like polypeptide derivatives” refers to TNFr/OPG-like polypeptides, fragments, or variants, as defined herein, that have been chemically modified. The derivatives are modified in a manner that is different from naturally occurring TNFr/OPG-like polypeptides, either in the type or location of the molecules attached to the polypeptide. Derivatives may further include molecules formed by the deletion of one or more chemical groups which are naturally attached to the TNFr/OPG-like polypeptide.

For example, the polypeptides may be modified by the covalent attachment of one or more polymers, including, but not limited to, water soluble polymers, N-linked or O-linked carbohydrates, sugars, phosphates, and/or other such molecules. For example, the polymer selected is typically water soluble so that the protein to which it is attached does not precipitate in an aqueous environment, such as a physiological environment. The polymer may be of any molecular weight, and may be branched or unbranched. Included within the scope of suitable polymers is a mixture of polymers. Preferably, for therapeutic use of the end-product preparation, the polymer will be pharmaceutically acceptable.

Suitable water soluble polymers or mixtures thereof include, but are not limited to, polyethylene glycol (PEG), monomethoxy-polyethylene glycol, dextran (such as low molecular weight dextran, of, for example about 6 kD), cellulose, or other carbohydrate based polymers, poly-(N-vinyl pyrrolidone) polyethylene glycol, propylene glycol homopolymers, a polypropylene oxide/ethylene oxide co-polymer, polyoxyethylated polyols (e.g., glycerol) and polyvinyl alcohol. Also encompassed by the present invention are bifunctional PEG crosslinking molecules which may be used to prepare covalently attached TNFr/OPG-like multimers.

For the acylation reactions, the polymer(s) selected should have a single reactive ester group. For reductive alkylation, the polymer(s) selected should have a single reactive aldehyde group. A reactive aldehyde is, for example, polyethylene glycol propionaldehyde, which is water stable, or mono C₁-C₁₀ alkoxy or aryloxy derivatives thereof (see U.S. Pat. No. 5,252,714).

The pegylation of TNFr/OPG-like polypeptides may be carried out by any of the pegylation reactions known in the art, as described for example in the following references: Francis et al., Focus on Growth Factors, 3:4-10 (1992); EP 0154316; EP 0401384 and U.S. Pat. No. 4,179,337. Pegylation may be carried out via an acylation reaction or an alkylation reaction with a reactive polyethylene glycol molecule (or an analogous reactive water-soluble polymer) as described herein.

Polyethylene glycol (PEG) is a water-soluble polymer suitable for use herein. As used herein, the terms “polyethylene glycol” and “PEG” are meant to encompass any of the forms of PEG that have been used to derivatize proteins, including mono-(C₁-C₁₀) alkoxy- or aryloxy-polyethylene glycol.

In general, chemical derivatization may be performed under any suitable conditions used to react a biologically active substance with an activated polymer molecule. Methods for preparing pegylated TNFr/OPG-like polypeptides will generally comprise the steps of (a) reacting the polypeptide with polyethylene glycol (such as a reactive ester or aldehyde derivative of PEG) under conditions whereby TNFr/OPG-like polypeptide becomes attached to one or more PEG groups, and (b) obtaining the reaction product(s). In general, the optimal reaction conditions for the acylation reactions will be determined based on known parameters and the desired result. For example, the larger the ratio of PEG:protein, the greater the percentage of poly-pegylated product. In one embodiment, the TNFr/OPG-like polypeptide derivative may have a single PEG moiety at the amino terminus. See, for example, U.S. Pat. No. 5,234,784.

Generally, conditions which may be alleviated or modulated by the administration of the present TNFr/OPG-like polypeptide derivative include those described herein. However, the TNFr/OPG-like polypeptide derivatives disclosed herein may have additional activities, enhanced or reduced biological activity, or other characteristics, such as increased or decreased half-life, as compared to the non-derivatized molecules.

The terms “biologically active TNFr/OPG-like polypeptides”, “biologically active TNFr/OPG-like polypeptide fragments”, “biologically active TNFr/OPG-like polypeptide variants”, and “biologically active TNFr/OPG-like polypeptide derivatives” refer to TNFr/OPG-like polypeptides having at least one activity characteristic of a TNFr/OPG-like polypeptide, such as the activity of the polypeptide set forth in either SEQ ID NO: 2 or SEQ ID NO: 4. In general, TNFr/OPG-like polypeptides, fragments, variants, and derivatives thereof, will have at least one activity characteristic of a TNFr/OPG-like polypeptide such as depicted in either SEQ ID NO: 2 or SEQ ID NO: 4. In addition, a TNFr/OPG-like polypeptide may be active as an immunogen, that is, the polypeptide contains at least one epitope to which antibodies may be raised.

“Naturally occurring” or “native” when used in connection with biological materials such as nucleic acid molecules, polypeptides, host cells, and the like, refers to materials which are found in nature and are not manipulated by man. Similarly, “non-naturally occurring” or “non-native” as used herein refers to a material that is not found in nature or that has been structurally modified or synthesized by man.

The term “isolated polypeptide” refers to a polypeptide of the present invention that is free from at least one contaminating polypeptide that is found in its natural environment. Preferably, the isolated polypeptide is substantially free from any other contaminating mammalian polypeptides which would interfere with its therapeutic, preventative, or diagnostic use.

The term “ortholog” refers to a polypeptide from another species that corresponds to TNFr/OPG-like polypeptide amino acid sequence as set forth in SEQ ID NOS: 2 or 4. For example, mouse and human TNFr/OPG-like polypeptides are considered orthologs of each other.

The term “mature TNFr/OPG-like polypeptide” refers to a polypeptide lacking a leader sequence. A mature polypeptide may also include other modifications such as proteolytic processing of the amino terminus (with or without a leader sequence) and/or the carboxy terminus, cleavage of a smaller polypeptide from a larger precursor, N-linked and/or O-linked glycosylation, and the like. An exemplary mature TNFr/OPG-like polypeptide is depicted by amino acid residue _ thorough amino acid residue of SEQ ID NO: 2 or amino acid residue through amino acid residue of SEQ ID NO: 4. FILL IN BLANKS

The terms “effective amount” and “therapeutically effective amount” refer to the amount of a TNFr/OPG-like polypeptide or TNFr/OPG-like nucleic acid molecule used to support an observable level of one or more biological activities of the TNFr/OPG-like polypeptides as set forth herein.

The term “selective binding agent” refers to a molecule or molecules having specificity for TNFr/OPG-like molecules. Selective binding agents include antibodies, such as polyclonal antibodies, monoclonal antibodies (mAbs), chimeric antibodies, CDR-grafted antibodies, anti-idiotypic (anti-Id) antibodies to antibodies that can be labeled in soluble or bound form, as well as fragments, regions, or derivatives thereof which are provided by known techniques, including, but not limited to enzymatic cleavage, peptide synthesis, or recombinant techniques. The anti-TNFr/OPG-like selective binding agents of the present invention are capable, for example, of binding portions of TNFr/OPG-like molecules that inhibit the binding of TNF/OPG-like molecules to TNFr/OPG-like receptors.

As used herein, the terms, “specific” and “specificity” refer to the ability of the selective binding agents to bind to human TNFr/OPG-like polypeptides and not to human non-TNFr/OPG-like polypeptides. It will be appreciated, however, that the selective binding agents may also bind orthologs of TNFr/OPG-like polypeptides, that is, interspecies versions of TNFr/OPG-like polypeptides, such as mouse and rat TNFr/OPG-like polypeptides. A preferred embodiment relates to antibodies that are highly specific to TNFr/OPG-like polypeptides yet do not cross-react (that is, they fail to bind) with specificity to non-TNFr/OPG-like polypeptides.

The term “antigen” refers to a molecule or a portion of a molecule capable of being bound by a selective binding agent, such as an antibody, which is additionally capable of inducing an animal to produce antibodies capable of binding to an epitope of that antigen. An antigen can have one or more epitopes. The specific binding reaction referred to above is meant to indicate that the antigen will react, in a highly selective manner, with its corresponding antibody and not with the multitude of other antibodies which can be evoked by other antigens.

TNFr/OPG-like polypeptides, fragments, variants, and derivatives may be used to prepare TNFr/OPG-like selective binding agents using methods known in the art. Thus, antibodies and antibody fragments that bind TNFr/OPG-like polypeptides are within the scope of the present invention. Antibody fragments include those portions of the antibody which bind to an epitope on the TNFr/OPG-like polypeptide. Examples of such fragments include Fab and F(ab′) fragments generated by enzymatic cleavage of full-length antibodies. Other binding fragments include those generated by recombinant DNA techniques, such as the expression of recombinant plasmids containing nucleic acid sequences encoding antibody variable regions. These antibodies may be, for example, polyclonal monospecific polyclonal, monoclonal, recombinant, chimeric, humanized, human, single chain, and/or bispecific.

Relatedness of Nucleic Acid Molecules and/or Polypeptides

It is understood that related nucleic acid molecules include allelic or splice variants of the nucleic acid molecule of SEQ ID NOS: 1 or 3, and include sequences which are complementary to any of the above nucleotide sequences. Related nucleic acid molecules also include a nucleotide sequence encoding a polypeptide comprising or consisting essentially of a substitution, modification, addition and/ora deletion of one or more amino acid residues compared to the polypeptide in SEQ ID NOS: 2 or 4.

Fragments include molecules which encode a polypeptide of at least about 25 amino acid residues, or about 50, or about 75, or about 100, or greater than about 100, amino acid residues of the polypeptide of SEQ ID NOS: 2 or 4.

In addition, related TNFr/OPG-like nucleic acid molecules include those molecules which comprise nucleotide sequences which hybridize under moderately or highly stringent conditions as defined herein with the fully complementary sequence of the nucleic acid molecule of SEQ ID NOS: 1 or 3, or of a molecule encoding a polypeptide, which polypeptide comprises the amino acid sequence as shown in SEQ ID NOS: 3 or 4, or of a nucleic acid fragment as defined herein, or of a nucleic acid fragment encoding a polypeptide as defined herein. Hybridization probes may be prepared using the TNFr/OPG-like sequences provided herein to screen cDNA, genomic or synthetic DNA libraries for related sequences. Regions of the DNA and/or amino acid sequence of TNFr/OPG-like polypeptide that exhibit significant identity to known sequences are readily determined using sequence alignment algorithms as described herein, and those regions may be used to design probes for screening.

The term “identity” as known in the art, refers to a relationship between the sequences of two or more polypeptide molecules or two or more nucleic acid molecules, as determined by comparing the sequences. In the art, “identity” also means the degree of sequence relatedness between nucleic acid molecule or polypeptide sequences, as the case may be, as determined by the match between strings of two or more nucleotide or two or more amino acid sequences. “Identity” measures the percent of identical matches between the smaller of two or more sequences with gap alignments (if any) addressed by a particular mathematical model or computer programs (i.e., “algorithms”).

The term “similarity” is a related concept, but in contrast to “identity”, refers to a measure of similarity which includes both identical matches and conservative substitution matches. If two polypeptide sequences have, for example, 10/20 identical amino acids, and the remainder are all non-conservative substitutions, then the percent identity and similarity would both be 50%. If in the same example, there are 5 more positions where there are conservative substitutions, then the percent identity remains 50%, but the per cent similarity would be 75% (15/20). Therefore, in cases where there are conservative substitutions, the degree of similarity between two polypeptide sequences will be higher than the percent identity between those two polypeptides.

In another embodiment, related nucleic acid molecules comprise or consist of a nucleotide sequence that is about 70 percent (70%) identical to the nucleotide sequence as shown in SEQ ID NOS: 1 or 3, or comprise or consist essentially of a nucleotide sequence encoding a polypeptide that is about 70 percent (70%) identical to the polypeptide as set forth in SEQ ID NOS: 2 or 4. In preferred embodiments, the nucleotide sequences are about 75 percent, or about 80 percent, or about 85 percent, or about 90 percent, or about 95, 96, 97, 98, or 99 percent identical to the nucleotide sequence as shown in SEQ ID NOS: 1 or 3, or the nucleotide sequences encode a polypeptide that is about 75 percent, or about 80 percent, or about 85 percent, or about 90 percent, or about 95, 96, 97, 98, or 99 percent identical to the polypeptide sequence as set forth in SEQ ID NOS: 2 or 4.

Differences in the nucleic acid sequence may result in conservative and/or non-conservative modifications of the amino acid sequence relative to the amino acid sequence of SEQ ID NOS: 2 or 4.

The term “conservative amino acid substitution” refers to a substitution of a native amino acid residue with a nonnative residue such that there is little or no effect on the polarity or charge of the amino acid residue at that position. For example, a conservative substitution results from the replacement of a non-polar residue in a polypeptide with any other non-polar residue. Furthermore, any native residue in the polypeptide may also be substituted with alanine, as has been previously described for “alanine scanning mutagenesis.” General rules for making amino acid substitutions are set forth in Table II.

TABLE II
Amino Acid Substitutions

| Original | Exemplary | Preferred |
|---|---|---|
| Residues | Substitutions | Substitutions |
| Ala | Val, Leu, Ile | Val |
| Arg | Lys, Gln, Asn | Lys |
| Asn | Gln | Gln |
| Asp | Glu | Glu |
| Cys | Ser, Ala | Ser |
| Gln | Asn | Asn |
| Glu | Asp | Asp |
| Gly | Pro, Ala | Ala |
| His | Asn, Gln, Lys, Arg | Arg |
| Ile | Leu, Val, Met, Ala, | Leu |
| | Phe, Norleucine | |
| Leu | Norleucine, Ile, | Ile |
| | Val, Met, Ala, Phe | |
| Lys | Arg, 1,4 Diamino- | Arg |
| | butyric Acid, Gln, | |
| | Asn | |
| Met | Leu, Phe, Ile | Leu |
| Phe | Leu, Val, Ile, Ala, | Leu |
| | Tyr | |
| Pro | Ala | Gly |
| Ser | Thr, Ala, Cys | Thr |
| Thr | Ser | Ser |
| Trp | Tyr, Phe | Tyr |
| Tyr | Trp, Phe, Thr, Ser | Phe |
| Val | Ile, Met, Leu, Phe, | Leu |
| | Ala, Norleucine | |

Conservative amino acid substitutions also encompass non-naturally occurring amino acid residues which are typically incorporated by chemical peptide synthesis rather than by synthesis in biological systems. These include peptidomimetics, and other reversed or inverted forms of amino acid moieties. It will be appreciated by those skilled in the art the nucleic acid and polypeptide molecules described herein may be chemically synthesized as well as produced by recombinant means.

Conservative modifications to the amino acid sequence (and the corresponding modifications to the encoding nucleotides) will produce TNFr/OPG-like polypeptides having functional and chemical characteristics similar to those of naturally occurring TNFr/OPG-like polypeptides. In contrast, substantial modifications in the functional and/or chemical characteristics of TNFr/OPG-like polypeptides may be accomplished by selecting substitutions that differ significantly in their effect on maintaining (a) the structure of the molecular backbone in the area of the substitution, for example, as a sheet or helical conformation, (b) the charge or hydrophobicity of the molecule at the target site, or (c) the bulk of the side chain. Naturally occurring residues may be divided into classes based on common side chain properties:

1) hydrophobic: norleucine, Met, Ala, Val, Leu, Ile;

2) neutral hydrophilic: Cys, Ser, Thr, Asn, Gln;

3) acidic: Asp, Glu;

4) basic: His, Lys, Arg;

5) residues that influence chain orientation: Gly, Pro; and

6) aromatic: Trp, Tyr, Phe.

Non-conservative substitutions may involve the exchange of a member of one of these classes for a member from another class. Such substituted residues may be introduced into regions of the human TNFr/OPG-like polypeptide that are homologous with non-human TNFr/OPG-like polypeptides, or into the non-homologous regions of the molecule.

Identity and similarity of related nucleic acid molecules and polypeptides can be readily calculated by known methods. Such methods include, but are not limited to, those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; Sequence Analysis Primer, Gribskov, M. and Devereux, J., eds., M. Stockton Press, New York, 1991; and Carillo et al., SIAM J. Applied Math., 48: 1073 (1988).

Preferred methods to determine identity and/or similarity are designed to give the largest match between the sequences tested. Methods to determine identity and similarity are described in publicly available computer programs. Preferred computer program methods to determine identity and similarity between two sequences include, but are not limited to, the GCG program package, including GAP (Devereux et al., Nucl. Acid. Res., 12: 387 (1984); Genetics Computer Group, University of Wisconsin, Madison, Wis.), BLASTP, BLASTN, and FASTA (Altschul et al., J. Mol. Biol., 215:403-410 (1990)). The BLASTX program is publicly available from the National Center for Biotechnology Information (NCBI) and other sources (BLAST Manual, Altschul et al. NCB/NLM/NIH Bethesda, Md. 20894; Altschul et al., supra). The well known Smith Waterman algorithm may also be used to determine identity.

Certain alignment schemes for aligning two amino acid sequences may result in the matching of only a short region of the two sequences, and this small aligned region may have very high sequence identity even though there is no significant relationship between the two full length sequences. Accordingly, in a preferred embodiment, the selected alignment method (GAP program) will result in an alignment that spans at least 50 contiguous amino acids of the target polypeptide.

For example, using the computer algorithm GAP (Genetics Computer Group, University of Wisconsin, Madison, Wis.), two polypeptides for which the percent sequence identity is to be determined are aligned for optimal matching of their respective amino acids (the “matched span”, as determined by the algorithm). A gap opening penalty (which is calculated as 3× the average diagonal; the “average diagonal” is the average of the diagonal of the comparison matrix being used; the “diagonal” is the score or number assigned to each perfect amino acid match by the particular comparison matrix) and a gap extension penalty (which is usually 1/10 times the gap opening penalty), as well as a comparison matrix such as PAM 250 or BLOSUM 62 are used in conjunction with the algorithm. A standard comparison matrix (see Dayhoff et al., Atlas of Protein Sequence and Structure, vol. 5, supp.3 (1978) for the PAM 250 comparison matrix; Henikoff et al., Proc. Natl. Acad. Sci USA, 89: 10915-10919 (1992) for the BLOSUM 62 comparison matrix) is also used by the algorithm.

Preferred parameters for a polypeptide sequence comparison include the following:

Algorithm: Needleman et al., J. Mol. Diol., 48, 443-453 (1970);

Comparison matrix: BLOSUM 62 from Henikoff et al., Proc. Natl. Acad. Sci. USA, 89: 10915-10919 (1992);

Gap Penalty: 12

Gap Length Penalty: 4

Threshold of Similarity: 0

The GAP program is useful with the above parameters. The aforementioned parameters are the default parameters for polypeptide comparisons (along with no penalty for end gaps) using the GAP algorithm.

Preferred parameters for nucleic acid molecule sequence comparisons include the following:

Algorithm: Needleman et al., J. Mol Biol., 48: 443-453 (1970);

Comparison matrix: matches=+10, mismatch=0

Gap Penalty: 50

Gap Length Penalty: 3

The GAP program is also useful with the above parameters. The aforementioned parameters are the default parameters for nucleic acid molecule comparisons.

Other exemplary algorithms, gap opening penalties, gap extension penalties, comparison matrices, thresholds of similarity, etc. may be used by those of skill in the art, including those set forth in the Program Manual, Wisconsin Package, Version 9, September, 1997. The particular choices to be made will be apparent to those of skill in the art and will depend on the specific comparison to be made, such as DNA to DNA, protein to protein, protein to DNA; and additionally, whether the comparison is between given pairs of sequences (in which case GAP or BestFit are generally preferred) or between one sequence and a large database of sequences (in which case FASTA or BLASTA are preferred).

Synthesis

It will appreciated by those skilled in the art the nucleic acid and polypeptide molecules described herein may be produced by recombinant and other means.

Nucleic Acid Molecules

The nucleic acid molecules encode a polypeptide comparing the amino acid sequence of a TNFr/OPG-like polypeptide can readily be obtained in a varienty of ways including, without limitation, chemical synthesis, cDNA or genomic library screening, expression library screening and/or PCR amplification of cDNA.

Recombinant DNA methods used herein are generally those set forth in Sambrook et al., Molecular Cloning: A Laboratory Manual, Cold Spring Harbor Laboratory Press, Cold Spring Harbor, N.Y. (1989), and/or Ausubel et al., eds., Current Protocols in Molecular Biology, Green Publishers Inc. and Wiley and Sons, NY (1994). The present invention provides for nucleic acid molecules as described herein and methods for obtaining the molecules.

A gene or cDNA encoding a TNFr/OPG-like polypeptide or fragment thereof may be obtained by hybridization screening of a genomic or cDNA library, or by PCR amplification. Where a gene encoding the amino acid sequence of a TNFr/OPG-like polypeptide has been identified from one species, all or a portion of that gene may be used as a probe to identify corresponding genes from other species (orthologs) or related genes from the same species (homologs). The probes or primers may be used to screen cDNA libraries from various tissue sources believed to express the TNFr/OPG-like polypeptide. In addition, part or all of a nucleic acid molecule having the sequence as set forth in either SEQ ID NOS: 1 or 3 may be used to screen a genomic library to identify and isolate a gene encoding the amino acid sequence of a TNFr/OPG-like polypeptide. Typically, conditions of moderate or high stringency will be employed for screening to minimize the number of false positives obtained from the screen.

Nucleic acid molecules encoding the amino acid sequence of TNFr/OPG-like polypeptides may also be identified by expression cloning which employs the detection of positive clones based upon a property of the expressed protein. Typically, nucleic acid libraries are screened by the binding of an antibody or other binding partner (e.g. receptor or ligand) to cloned proteins which are expressed and displayed on a host cell surface. The antibody or binding partner is modified with a detectable label to identify those cells expressing the desired clone.

Recombinant expression techniques conducted in accordance with the descriptions set forth below may be followed to produce these polynucleotides and to express the encoded polypeptides. For example, by inserting a nucleic acid sequence which encodes the amino acid sequence of a TNFr/OPG-like polypeptide into an appropriate vector, one skilled in the art can readily produce large quantities of the desired nucleotide sequence. The sequences can then be used to generate detection probes or amplification primers. Alternatively, a polynucleotide encoding the amino acid sequence of an TNFr/OPG-like polypeptide can be inserted into an expression vector. By introducing the expression vector into an appropriate host, the encoded TNFr/OPG-like polypeptide may be produced in large amounts.

Another method for obtaining a suitable nucleic acid sequence is the polymerase chain reaction (PCR). In this method, cDNA is prepared from poly(A)+RNA or total RNA using the enzyme reverse transcriptase. Two primers, typically complementary to two separate regions of cDNA (oligonucleotides) encoding the amino acid sequence of an TNFr/OPG-like polypeptide, are then added to the cDNA along with a polymerase such as Taq polymerase, and the polymerase amplifies the cDNA region between the two primers.

Another means of preparing a nucleic acid molecule encoding the amino acid sequence of a TNFr/OPG-like polypeptide, including a fragment or variant, is chemical synthesis using methods well known to the skilled artisan such as those described by Engels et al., Angew. Chem. Intl. Ed., 28: 716-734 (1989). These methods include, inter alia, the phosphotriester, phosphoramidite, and H-phosphonate methods for nucleic acid synthesis. A preferred method for such chemical synthesis is polymer-supported synthesis using standard phosphoramidite chemistry. Typically, the DNA encoding the amino acid sequence of a TNFr/OPG-like polypeptide will be several hundred nucleotides in length. Nucleic acids larger than about 100 nucleotides can be synthesized as several fragments using these methods. The fragments can then be ligated together to form the full length nucleotide sequence of a TNFr/OPG-like polypeptide. Usually, the DNA fragment encoding the amino terminus of the polypeptide will have an ATG, which encodes a methionine residue. This methionine may or may not be present on the mature form of the TNFr/OPG-like polypeptide, depending on whether the polypeptide produced in the host cell is designed to be secreted from that cell.

In some cases, it may be desirable to prepare nucleic acid molecules encoding TNFr/OPG-like polypeptide variants. Nucleic acid molecules encoding variants may be produced using site directed mutagenesis, PCR amplification, or other appropriate methods, where the primer(s) have the desired point mutations (see Sambrook et al., supra, and Ausubel et al., supra, for descriptions of mutagenesis techniques). Chemical synthesis using methods described by Engels et al., supra, may also be used to prepare such variants. Other methods known to the skilled artisan may be used as well.

In certain embodiments, nucleic acid variants contain codons which have been altered for the optimal expression of a TNFr/OPG-like polypeptide in a given host cell. Particular codon alterations will depend upon the TNFr/OPG-like polypeptide(s) and host cell(s) selected for expression. Such “codon optimization” can be carried out by a variety of methods, for example, by selecting codons which are preferred for use in highly expressed genes in a given host cell. Computer algorithms which incorporate codon frequency tables such as “Ecohigh.cod” for codon preference of highly expressed bacterial genes may be used and are provided by the University of Wisconsin Package Version 9.0, Genetics Computer Group, Madison, Wis. Other useful codon frequency tables include “Celegans_high.cod”, “Celegans_low.cod”, “Drosophila_high.cod”, “Human_high.cod”, “Maize_high.cod”, and “Yeast_high.cod”.

In other embodiments, nucleic acid molecules encode TNFr/OPG-like variants with conservative amino acid substitutions as described herein, TNFr/OPG-like variants comprising an addition and/or a deletion of one or more N-linked or O-linked glycosylation sites, TNFr/OPG-like variants having deletions and/or substitutions of one or more cysteine residues, or TNFr/OPG-like polypeptide fragments as described herein. In addition, nucleic acid molecules may encode any combination of TNFr/OPG-like variants, fragments, and fusion polypeptides described herein.

Vectors and Host Cells

A nucleic acid molecule encoding the amino acid sequence of a TNFr/OPG-like polypeptide is inserted into an appropriate expression vector using standard ligation techniques. The vector is typically selected to be functional in the particular host cell employed (i.e., the vector is compatible with the host cell machinery such that amplification of the gene and/or expression of the gene can occur). A nucleic acid molecule encoding the amino acid sequence of a TNFr/OPG-like polypeptide may be amplified/expressed in prokaryotic, yeast, insect (baculovirus systems), and/or eukaryotic host cells. Selection of the host cell will depend in part on whether a TNFr/OPG-like polypeptide is to be post-translationally modified (e.g., glycosylated and/or phosphorylated). If so, yeast, insect, or mammalian host cells are preferable. For a review of expression vectors, see Meth. Enz., v.185, D. V. Goeddel, ed. Academic Press Inc., San Diego, Calif. (1990).

Typically, expression vectors used in any of the host cells will contain sequences for plasmid maintenance and for cloning and expression of exogenous nucleotide sequences. Such sequences, collectively referred to as “flanking sequences” in certain embodiments will typically include one or more of the following nucleotide sequences: a promoter, one or more enhancer sequences, an origin of replication, a transcriptional termination sequence, a complete intron sequence containing a donor and acceptor splice site,a sequence encoding a leader sequence for polypeptide secretion, a ribosome binding site, a polyadenylation sequence, a polylinker region for inserting the nucleic acid encoding the polypeptide to be expressed, and a selectable marker element. Each of these sequences is discussed below.

Optionally, the vector may contain a “tag”-encoding sequence, i.e., an oligonucleotide molecule located at the 5′ or 3′ end of the TNFr/OPG-like polypeptide coding sequence; the oligonucleotide sequence encodes polyHis (such as hexaHis), or other “tag” such as FLAG, HA (hemaglutinin Influenza virus) or myc for which commercially available antibodies exist. This tag is typically fused to the polypeptide upon expression of the polypeptide, and can serve as a means for affinity purification of the TNFr/OPG-like polypeptide from the host cell. Affinity purification can be accomplished, for example, by column chromatography using antibodies against the tag as an affinity matrix. Optionally, the tag can subsequently be removed from the purified TNFr/OPG-like polypeptide by various means such as using certain peptidases for cleavage.

Flanking sequences may be homologous (i.e., from the same species and/or strain as the host cell), heterologous (i.e., from a species other than the host cell species or strain), hybrid (i.e., a combination of flanking sequences from more than one source), or synthetic, or the flanking sequences may be native sequences which normally function to regulate TNFr/OPG-like polypeptide expression. As such, the source of a flanking sequence may be any prokaryotic or eukaryotic organism, any vertebrate or invertebrate organism, or any plant, provided that the flanking sequences is functional in, and can be activated by, the host cell machinery.

The flanking sequences useful in the vectors of this invention may be obtained by any of several methods well known in the art. Typically, flanking sequences useful herein other than endogenous TNFr/OPG-like gene flanking sequences will have been previously identified by mapping and/or by restriction endonuclease digestion and can thus be isolated from the proper tissue source using the appropriate restriction endonucleases. In some cases, the full nucleotide sequence of a flanking sequence may be known. Here, the flanking sequence may be synthesized using the methods described herein for nucleic acid synthesis or cloning.

Where all or only a portion of the flanking sequence is known, it may be obtained using PCR and/or by screening a genomic library with suitable oligonucleotide and/or flanking sequence fragments from the same or another species. Where the flanking sequence is not known, a fragment of DNA containing a flanking sequence may be isolated from a larger piece of DNA that may contain, for example, a coding sequence or even another gene or genes. Isolation may be accomplished by restriction endonuclease digestion to produce the proper DNA fragment followed by isolation using agarose gel purification, Qiagen® column chromatography (Chatsworth, Calif.), or other methods known to the skilled artisan. The selection of suitable enzymes to accomplish this purpose will be readily apparent to one of ordinary skill in the art.

An origin of replication is typically a part of those prokaryotic expression vectors purchased commercially, and the origin aids in the amplification of the vector in a host cell. Amplification of the vector to a certain copy number can, in some cases, be important for the optimal expression of the TNFr/OPG-like polypeptide. If the vector of choice does not contain an origin of replication site, one may be chemically synthesized based on a known sequence, and ligated into the vector. For example, the origin of replication from the plasmid pBR322 (Product No. 303-3s, New England Biolabs, Beverly, Mass.) is suitable for most Gram-negative bacteria and various origins (e.g., SV40, polyoma, adenovirus, vesicular stomatitus virus (VSV) or papillomaviruses such as HPV or BPV) are useful for cloning vectors in mammalian cells. Generally, the origin of replication component is not needed for mammalian expression vectors (for example, the SV40 origin is often used only because it contains the early promoter).

A transcription termination sequence is typically located 3′ of the end of a polypeptide coding region and serves to terminate transcription. Usually, a transcription termination sequence in prokaryotic cells is a G-C rich fragment followed by a poly T sequence. While the sequence is easily cloned from a library or even purchased commercially as part of a vector, it can also be readily synthesized using methods for nucleic acid synthesis such as those described herein.

A selectable marker gene element encodes a protein necessary for the survival and growth of a host cell grown in a selective culture medium. Typical selection marker genes encode proteins that (a) confer resistance to antibiotics or other toxins, e.g., ampicillin, tetracycline, or kanamycin for prokaryotic host cells, (b) complement auxotrophic deficiencies of the cell; or (c) supply critical nutrients not available from complex media. Preferred selectable markers are the kanamycin resistance gene, the ampicillin resistance gene, and the tetracycline resistance gene. A neomycin resistance gene may also be used for selection in prokaryotic and eukaryotic host cells.

Other selection genes may be used to amplify the gene which will be expressed. Amplification is the process wherein genes which are in greater demand for the production of a protein critical for growth are reiterated in tandem within the chromosomes of successive generations of recombinant cells. Examples of suitable selectable markers for mammalian cells include dihydrofolate reductase (DHFR) and thymidine kinase. The mammalian cell transformants are placed under selection pressure which only the transformants are uniquely adapted to survive by virtue of the selection gene present in the vector. Selection pressure is imposed by culturing the transformed cells under conditions in which the concentration of selection agent in the medium is successively changed, thereby leading to the amplification of both the selection gene and the DNA that encodes TNFr/OPG-like polypeptides. As a result, increased quantities of TNFr/OPG-like polypeptides are synthesized from the amplified DNA.

A ribosome binding site is usually necessary for translation initiation of mRNA and is characterized by a Shine-Dalgarno sequence (prokaryotes) or a Kozak sequence (eukaryotes). The element is typically located 3′ to the promoter and 5′ to the coding sequence of the TNFr/OPG-like polypeptide to be expressed. The Shine-Dalgarno sequence is varied but is typically a polypurine (i.e., having a high A-G content). Many Shine-Dalgarno sequences have been identified, each of which can be readily synthesized using methods set forth herein and used in a prokaryotic vector.

A leader, or signal, sequence may be used to direct a TNFr/OPG-like polypeptide out of the host cell. Typically, a nucleotide sequence encoding the signal sequence is positioned in the coding region of the TNFr/OPG-like nucleic acid molecule, or directly at the 5′ end of the TNFr/OPG-like polypeptide coding region. Many signal sequences have been identified, and any of those that are functional in the selected host cell may be used in conjunction with the TNFr/OPG-like nucleic acid molecule. Therefore, a signal sequence may be homologous (naturally occurring) or heterologous to the TNFr/OPG-like gene or cDNA. Additionally, a signal sequence may be chemically synthesized using methods described herein. In most cases, the secretion of a TNFr/OPG-like polypeptide from the host cell via the presence of a signal peptide will result in the removal of the signal peptide from the secreted TNFr/OPG-like polypeptide. The signal sequence may be a component of the vector, or it may be a part of TNFr/OPG-like nucleic acid molecule that is inserted into the vector.

Included within the scope of this invention is the use of either nucleotide sequence encoding a native TNFr/OPG-like signal sequence joined to a TNFr/OPG-like polypeptide coding region or a nucleotide sequence encoding a a heterologous signal sequence joined to a TNFr/OPG-like polypeptide coding region. The heterologous signal sequence selected should be one that is recognized and processed, i.e., cleaved by a signal peptidase, by the host cell. For prokaryotic host cells that do not recognize and process the native TNFr/OPG-like polypeptide signal sequence, the signal sequence is substituted by a prokaryotic signal sequence selected, for example, from the group of the alkaline phosphatase, penicillinase, or heat-stable enterotoxin II leaders. For yeast secretion, the native TNFr/OPG-like polypeptide signal sequence may be substituted by the yeast invertase, alpha factor, or acid phosphatase leaders. In mammalian cell expression the native signal sequence is satisfactory, although other mammalian signal sequences may be suitable.

In some cases, such as where glycosylation is desired in a eukaryotic host cell expression system, one may manipulate the various presequences to improve glycosylation or yield. For example, one may alter the peptidase cleavage site of a particular signal peptide, or add presequences, which also may affect glycosylation. The final protein product may have, in the −1 position (relative to the first amino acid of the mature protein) one or more additional amino acids incident to expression, which may not have been totally removed. For example, the final protein product may have one or two amino acid residues found in the peptidase cleavage site, attached to the N-terminus. Alternatively, use of some enzyme cleavage sites may result in a slightly truncated form of the desired TNFr/OPG-like polypeptide, if the enzyme cuts at such area within the mature polypeptide.

In many cases, transcription of a nucleic acid molecule is increased by the presence of one or more introns in the vector; this is particularly true where a polypeptide is produced in eukaryotic host cells, especially mammalian host cells. The introns used may be naturally occurring within the TNFr/OPG-like gene, especially where the gene used is a full length genomic sequence or a fragment thereof. Where the intron is not naturally occurring within the gene (as for most cDNAs), the intron(s) may be obtained from another source. The position of the intron with respect to flanking sequences and the TNFr/OPG-like gene is generally important, as the intron must be transcribed to be effective. Thus, when a TNFr/OPG-like cDNA molecule is being transcribed, the preferred position for the intron is 3′ to the transcription start site, and 5′ to the polyA transcription termination sequence. Preferably, the intron or introns will be located on one side or the other (i.e., 5′ or 3′) of the cDNA such that it does not interrupt the coding sequence. Any intron from any source, including any viral, prokaryotic and eukaryotic (plant or animal) organisms, may be used to practice this invention, provided that it is compatible with the host cell(s) into which it is inserted. Also included herein are synthetic introns. optionally, more than one intron may be used in the vector.

The expression and cloning vectors of the present invention will each typically contain a promoter that is recognized by the host organism and operably linked to the molecule encoding a TNFr/OPG-like polypeptide. Promoters are untranscribed sequences located upstream (5′) to the start codon of a structural gene (generally within about 100 to 1000 bp) that control the transcription and translation of the structural gene. Promoters are conventionally grouped into one of two classes, inducible promoters and constitutive promoters. Inducible promoters initiate increased levels of transcription from DNA under their control in response to some change in culture conditions, such as the presence or absence of a nutrient or a change in temperature. Constitutive promoters, on the other hand, initiate continual gene product production; that is, there is little or no control over gene expression. A large number of promoters, recognized by a variety of potential host cells, are well known. A suitable promoter is operably linked to the DNA encoding a TNFr/OPG-like polypeptide by removing the promoter from the source DNA by restriction enzyme digestion and inserting the desired promoter sequence into the vector. The native TNFr/OPG-like gene promoter sequence may be used to direct amplification and/or expression of TNFr/OPG-like nucleic acid molecule. A heterologous promoter is preferred, however, if it permits greater transcription and higher yields of the expressed protein as compared to the native promoter, and if it is compatible with the host cell system that has been selected for use.

Promoters suitable for use with prokaryotic hosts include the beta-lactamase and lactose promoter systems; alkaline phosphatase, a tryptophan (trp) promoter system; and hybrid promoters such as the tac promoter. Other known bacterial promoters are also suitable. Their sequences have been published, thereby enabling one skilled in the art to ligate them to the desired DNA sequence(s), using linkers or adapters as needed to supply any useful restriction sites.

Suitable promoters for use with yeast hosts are also well known in the art. Yeast enhancers are advantageously used with yeast promoters. Suitable promoters for use with mammalian host cells are well known and include, but are not limited to, those obtained from the genomes of viruses such as polyoma virus, fowlpox virus, adenovirus (such as Adenovirus 2), bovine papilloma virus, avian sarcoma virus, cytomegalovirus (CMV), a retrovirus, hepatitis-B virus and most preferably Simian Virus 40 (SV40). Other suitable mammalian promoters include heterologous mammalian promoters, e.g., heat-shock promoters and the actin promoter.

Additional promoters which may be of interest in controlling TNFr/OPG-like gene transcription include, but are not limited to: the SV40 early promoter region (Bernoist and Chambon, Nature, 290:304-310, 1981); the CMV promoter; the promoter contained in the 3′ long terminal repeat of Rous sarcoma virus (Yamamoto et al., Cell, 22: 787-797, 1980); the herpes thymidine kinase promoter (Wagner et al., Proc. Natl. Acad. Sci. USA, 78: 144-1445, 1981); the regulatory sequences of the metallothionine gene (Brinster et al., Nature, 296: 39-42, 1982); prokaryotic expression vectors such as the beta-lactamase promoter (Villa-Kamaroff, et al., Proc. Natl. Acad. Sci. USA, 75:3727-3731, 1978); or the tac promoter (DeBoer, et al., Proc. Natl. Acad. Sci. USA, 80:21-25, 1983). Also of interest are the following animal transcriptional control regions, which exhibit tissue specificity and have been utilized in transgenic animals: the elastase I gene control region which is active in pancreatic acinar cells (Swift et al., Cell, 38: 639-646, 1984; Ornitz et al., Cold Spring Harbor Symp. Quant. Biol., 50:399-409 (1986); MacDonald, Hepatology, 7:425-515, 1987); the insulin gene control region which is active in pancreatic beta cells (Hanahan, Nature, 315:115-122, 1985); the immunoglobulin gene control region which is active in lymphoid cells (Grosschedl et al., Cell, 38:647-658 (1984); Adames et al., Nature, 318:533-538 (1985); Alexander et al., Mol. Cell. Biol., 7:1436-1444, 1987); the mouse mammary tumor virus control region which is active in testicular, breast, lymphoid and mast cells (Leder et al., Cell, 45:485-495, 1986); the albumin gene control region which is active in liver (Pinkert et al., Genes and Devel., 1:268-276, 1987); the alphafetoprotein gene control region which is active in liver (Krumlauf et al., Mol. Cell. Biol., 5:1639-1648, 1985; Hammer et al., Science, 235:53-58, 1987); the alpha 1-antitrypsin gene control region which is active in the liver (Kelsey et al., Genes and Devel., 1:161-171, 1987); the beta-globin gene control region which is active in myeloid cells (Mogram et al., Nature, 315:338-340, 1985; Kollias et al., Cell, 46:89-94, 1986); the myelin basic protein gene control region which is active in oligodendrocyte cells in the brain (Readhead et al., Cell, 48:703-712, 1987); the myosin light chain-2 gene control region which is active in skeletal muscle (Sani, Nature, 314:283-286, 1985); and the gonadotropic releasing hormone gene control region which is active in the hypothalamus (Mason et al., Science, 234:1372-1378, 1986).

An enhancer sequence may be inserted into the vector to increase the transcription of a DNA encoding a TNFr/OPG-like polypeptide of the present invention by higher eukaryotes. Enhancers are cis-acting elements of DNA, usually about 10-300 bp in length, that act on the promoter to increase transcription. Enhancers are relatively orientation and position independent. They have been found 5′ and 3′ to the transcription unit. Several enhancer sequences available from mammalian genes are known (e.g., globin, elastase, albumin, alpha-feto-protein and insulin). Typically, however, an enhancer from a virus will be used. The SV40 enhancer, the cytomegalovirus early promoter enhancer, the polyoma enhancer, and adenovirus enhancers are exemplary enhancing elements for the activation of eukaryotic promoters. While an enhancer may be spliced into the vector at a position 5′ or 3′ to TNFr/OPG-like nucleic acid molecule, it is typically located at a site 5′ from the promoter.

Expression vectors of the invention may be constructed from a starting vector such as a commercially available vector. Such vectors may or may not contain all of the desired flanking sequences. Where one or more of the desired flanking sequences are not already present in the vector, they may be individually obtained and ligated into the vector. Methods used for obtaining each of the flanking sequences are well known to one skilled in the art.

Preferred vectors for practicing this invention are those which are compatible with bacterial, insect, and mammalian host cells. Such vectors include, inter alia, PCRII, pCR3, and pcDNA3.1 (Invitrogen Company, Carlsbad, Calif.), pBSII (Stratagene Company, La Jolla, Calif.), pET15 (Novagen, Madison, Wis.), pGEX (Pharmacia Biotech, Piscataway, N.J.), pEGFP-N2 (Clontech, Palo Alto, Calif.), pETL (BlueBacII; Invitrogen), PDSR-alpha (PCT Publication No. WO90/14363) and pFastBacDual (Gibco/BRL, Grand Island, N.Y.).

Additional suitable vectors include, but are not limited to, cosmids, plasmids or modified viruses, but it will be appreciated that the vector system must be compatible with the selected host cell. Such vectors include, but are not limited to plasmids such as Bluescript® plasmid derivatives (a high copy number ColE1-based phagemid, Stratagene Cloning Systems Inc., La Jolla Calif.), PCR cloning plasmids designed for cloning Taq-amplified PCR products (e.g., TOPO™ TA Cloning® Kit, PCR2.1® plasmid derivatives, Invitrogen, Carlsbad, Calif.), and mammalian, yeast, or virus vectors such as a baculovirus expression system (pBacPAK plasmid derivatives, Clontech, Palo Alto, Calif.). The recombinant molecules can be introduced into host cells via transformation, transfection, infection, electroporation, or other known techniques.

After the vector has been constructed and a nucleic acid molecule encoding a TNFr/OPG-like polypeptide has been inserted into the proper site of the vector, the completed vector may be inserted into a suitable host cell for amplification and/or polypeptide expression. Host cells may be prokaryotic host cells (such as E. coli) or eukaryotic host cells (such as a yeast cell, an insect cell, or a vertebrate cell). The host cell, when cultured under appropriate conditions, synthesizes a TNFr/OPG-like polypeptide which can subsequently be collected from the culture medium (if the host cell secretes it into the medium) or directly from the host cell producing it (if it is not secreted). The selection of an appropriate host cell will depend upon various factors, such as desired expression levels, polypeptide modifications that are desirable or necessary for activity, such as glycosylation or phosphorylation, and ease of folding into a biologically active molecule.

A number of suitable host cells are known in the art and many are available from the American Type Culture Collection (ATCC), 10801 University Boulevard Manassas, Va. 20110-2209. Examples include, but are not limited to, mammalian cells, such as Chinese hamster ovary cells (CHO) (ATCC No. CCL61) CHO DHFR-cells (Urlaub et al., Proc. Natl. Acad. Sci. USA, 97:4216-4220 (1980)), human embryonic kidney (HEK) 293 or 293T cells (ATCC No. CRL1573), or 3T3 cells (ATCC No. CCL92). The selection of suitable mammalian host cells and methods for transformation, culture, amplification, screening and product production and purification are known in the art. Other suitable mammalian cell lines, are the monkey COS-1 (ATCC No. CRL1650) and COS-7 cell lines (ATCC No. CRL1651), and the CV-1 cell line (ATCC No. CCL70). Further exemplary mammalian host cells include primate cell lines and rodent cell lines, including transformed cell lines. Normal diploid cells, cell strains derived from in vitro culture of primary tissue, as well as primary explants, are also suitable. Candidate cells may be genotypically deficient in the selection gene, or may contain a dominantly acting selection gene. Other suitable mammalian cell lines include but are not limited to, mouse neuroblastoma N2A cells, HeLa, mouse L-929 cells, 3T3 lines derived from Swiss, Balb-c or NIH mice, BHK or HaK hamster cell lines, which are available from the ATCC). Each of these cell lines is known by and available to those skilled in the art of protein expression.

Similarly useful as host cells suitable for the present invention are bacterial cells. For example, the various strains of E. coli (e.g., HB101, (ATCC No. 33694) DH5α, DH10, and MC1061 (ATCC No. 53338)) are well-known as host cells in the field of biotechnology. Various strains of B. subtilis, Pseudomonas spp., other Bacillus spp., Streptomyces spp., and the like may also be employed in this method.

Many strains of yeast cells known to those skilled in the art are also available as host cells for the expression of the polypeptides of the present invention. Preferred yeast cells include, for example, Saccharomyces cerivisae and Pichia pastoris.

Additionally, where desired, insect cell systems may be utilized in the methods of the present invention. Such systems are described for example in Kitts et al., Biotechniques, 14:810-817 (1993); Lucklow, Curr. Opin. Biotechnol., 4:564-572 (1993); and Lucklow et al. (J. Virol., 67:4566-4579 (1993). Preferred insect cells are Sf-9 and Hi5 (Invitrogen, Carlsbad, Calif.).

The transformation of an expression vector for a TNFr/OPG-like polypeptide into a selected host cell may be accomplished by well known methods including methods such as calcium chloride, electroporation, microinjection, lipofection or the DEAE-dextran method. The method selected will in part be a function of the type of host cell to be used. These methods and other suitable methods are well known to the skilled artisan, and are set forth, for example, in Sambrook et al., supra.

One may also use transgenic animals to express glycosylated TNFr/OPG-like polypeptides. For example, one may use a transgenic milk-producing animal (a cow or goat, for example) and obtain the present glycosylated polypeptide in the animal milk. One may also use plants to produce TNfr/OPG-like polypeptides, however, in general, the glycosylation occurring in plants is different from that produced in mammalian cells, and may result in a glycosylated product which is not suitable for human therapeutic use.

Polypeptide Production

Host cells comprising a TNFr/OPG-like polypeptide expression vector may be cultured using standard media well known to the skilled artisan. The media will usually contain all nutrients necessary for the to allow growth and survival of the cells. Suitable media for culturing E. coli cells include for example, Luria Broth (LB) and/or Terrific Broth (TB). Suitable media for culturing eukaryotic cells include Roswell Park Memorial Institute medium 1640 (RPMI 1640), Minimal Essential Medium (MEM) and/or Dulbecco's Modified Eagle Medium (DMEM), all of which may be supplemented with serum and/or growth factors as indicated by the particular cell line being cultured. A suitable medium for insect cultures is Grace's medium supplemented with yeastolate, lactalbumin hydrolysate, and/or fetal calf serum, as necessary.

Typically, an antibiotic or other compound useful for selective growth of transformed cells is added as a supplement to the media. The compound to be used will be dictated by the selectable marker element present on the plasmid with which the host cell was transformed. For example, where the selectable marker element is kanamycin resistance, the compound added to the culture medium will be kanamycin. Other compounds for selective growth include ampicillin, tetracycline, and neomycin.

The amount of a TNFr/OPG-like polypeptide produced by a host cell can be evaluated using standard methods known in the art. Such methods include, without limitation, Western blot analysis, SDS-polyacrylamide gel electrophoresis, non-denaturing gel electrophoresis, High Performance Liquid Chromatography (HPLC) separation, immunoprecipitation, and/or activity assays such as DNA binding gel shift assays.

If a TNFr/OPG-like polypeptide has been designed to be secreted from the host cells, the majority of polypeptide may be found in the cell culture medium. If however, the TNFr/OPG-like polypeptide is not secreted from the host cells, it will be present in the cytoplasm and/or the nucleus (for eukaryotic host cells) or in the cytosol (for bacterial host cells).

For a TNFr/OPG-like polypeptide situated in the host cell cytoplasm and/or the nucleus (for eukaryotic host cells) or in the cytosol (for bacterial host cells), the host cells are typically first disrupted mechanically or with a detergent to release the intra-cellular contents into a buffered solution. TNFr/OPG-like polypeptide can then be isolated from this solution.

The purification of a TNFr/OPG-like polypeptide from solution can be accomplished using a variety of techniques. If the polypeptide has been synthesized such that it contains a tag such as Hexahistidine (TNFr/OPG-like polypeptide/hexaHis) or other small peptide such as FLAG (Eastman Kodak Co., New Haven, Conn.) or myc (Invitrogen, Carlsbad, Calif.) at either its carboxyl or amino terminus, it may essentially be purified in a one-step process by passing the solution through an affinity column where the column matrix has a high affinity for the tag. For example, polyhistidine binds with great affinity and specificity to nickel, thus an affinity column of nickel (such as the Qiagen® nickel columns) can be used for purification of TNFr/OPG-like polypeptide/polyHis. See for example, Ausubel et al., eds., Current Protocols in Molecular Biology, Section 10.11.8, John Wiley & Sons, New York (1993).

Additionally, the TNFr/OPG-like polypeptide may be purified through the use of a monoclonal antibody whih is capable of specifically recognizing and binding to the TNFr/OPG-like polypeptide.

Where a TNFr/OPG-like polypeptide is prepared without a tag attached, and no antibodies are available, other well known procedures for purification can be used. Such procedures include, without limitation, ion exchange chromatography, molecular sieve chromatography, High Performance Liquid Chromatography (HPLC), native gel electrophoresis in combination with gel elution, and preparative isoelectric focusing (“Isoprime” machine/technique, Hoefer Scientific, San Francisco, Calif.). In some cases, two or more of these techniques may be combined to achieve increased purity.

If a TNFr/OPG-like polypeptide is produced intracellularly, the intracellular material (including inclusion bodies for gram-negative bacteria) can be extracted from the host cell using any standard technique known to the skilled artisan. For example, the host cells can be lysed to release the contents of the periplasm/cytoplasm by French press, homogenization, and/or sonication followed by centrifugation.

If a TNFr/OPG-like polypeptide has formed inclusion bodies in the cytosol, the inclusion bodies can often bind to the inner and/or outer cellular membranes and thus will be found primarily in the pellet material after centrifugation. The pellet material can then be treated at pH extremes or with a chaotropic agent such as a detergent, guanidine, guanidine derivatives, urea, or urea derivatives in the presence of a reducing agent such as dithiothreitol at alkaline pH or tris carboxyethyl phosphine at acid pH to release, break apart, and solubilize the inclusion bodies. This solubilized TNFr/OPG-like polypeptide in its now soluable form can then be analyzed using gel electrophoresis, immunoprecipitation or the like. If it is desired to isolate the TNFr/OPG-like polypeptide, isolation may be accomplished using standard methods such as those described herein and in Marston et al., Meth. Enz., 182:264-275 (1990).

In some cases, a TNFr/OPG-like polypeptide may not be biologically active upon isolation. Various methods for “refolding” or converting the polypeptide to its tertiary structure and generating disulfide linkages, can be used to restore biological activity. Such methods include exposing the solubilized polypeptide to a pH usually above 7 and in the presence of a particular concentration of a chaotrope. The selection of chaotrope is very similar to the choices used for inclusion body solubilization, but usually the chaotrope is used at a lower concentration and is not necessarily the same as chaotropes used for the solubilization. In most cases the refolding/oxidation solution will also contain a reducing agent or the reducing agent plus its oxidized form in a specific ratio to generate a particular redox potential allowing for disulfide shuffling to occur in the formation of the protein's cysteine bridge(s). Some of the commonly used redox couples include cysteine/cystamine, glutathione (GSH)/dithiobis GSH, cupric chloride, dithiothreitol(DTT)/dithiane DTT, and 2-2mercaptoethanol(bME)/dithio-b(ME). A cosolvent may be used to increase the efficiency of the refolding, and the more common reagents used for this purpose include glycerol, polyethylene glycol of various molecular weights, arginine and the like.

If inclusion bodies are not formed to a significant degree upon expression of a TNFr/OPG-like polypeptide, then the polypeptide will be found primarily in the supernatant after centrifugation of the cell homogenate. The polypeptide may be further isolated from the supernatant using methods such as those described herein.

Suitable procedures for purification thus include, without limitation, affinity chromatography, immunoaffinity chromatography, ion exchange chromatography, molecular sieve chromatography, High Performance Liquid Chromatography (HPLC), electrophoresis (including native gel electrophoresis) followed by gel elution, and preparative isoelectric focusing (“Isoprime” machine/technique, Hoefer Scientific, San Francisco, Calif.). In some cases, two or more purification techniques may be combined to achieve increased purity.

TNFr/OPG-like polypeptides, including fragments, variants, and/or derivatives thereof may also be prepared by chemical synthesis methods (such as solid phase peptide synthesis) using techniques known in the art, such as those set forth by Merrifield et al., J. Am. Chem. Soc., 85:2149 (1963), Houghten et al., Proc Natl Acad. Sci. USA, 82:5132 (1985), and Stewart and Young, Solid Phase Peptide Synthesis, Pierce Chemical Co., Rockford, Ill. (1984). Such polypeptides may be synthesized with or without a methionine on the amino terminus. Chemically synthesized TNFr/OPG-like polypeptides may be oxidized using methods set forth in these references to form disulfide bridges. Chemically synthesized TNFr/OPG-like polypeptides are expected to have comparable biological activity to the corresponding TNFr/OPG-like polypeptides produced recombinantly or purified from natural sources, and thus may be used interchangeably with a recombinant or natural TNFr/OPG-like polypeptide.

Another means of obtaining a TNFr/OPG-like polypeptide is via purification from biological samples such as source tissues and/or fluids in which the TNFr/OPG-like polypeptide is naturally found. Such purification can be conducted using methods for protein purification as described herein. The presence of the TNFr/OPG-like polypeptide during purification may be monitored using, for example, an antibody prepared against recombinantly produced TNFr/OPG-like polypeptide or peptide fragments thereof.

A number of additional methods for producing nucleic acids and polypeptides are known in the art, and the methods can be used to produce polypeptides having specificity for TNFr/OPG-like. See for example, Roberts et al., Proc. Natl. Acad. Sci. USA, 94:12297-12303 (1997), which describes the production of fusion proteins between an mRNA and its encoded peptide. See also Roberts, R., Curr. Opin. Chem. Biol., 3:268-273 (1999). Additionally, U.S. Pat. No. 5,824,469 describes methods of obtaining oligonucleotides capable of carrying out a specific biological function. The procedure involves generating a heterogeneous pool of oligonucleotides, each having a 5′ randomized sequence, a central preselected sequence, and a 3′ randomized sequence. The resulting heterogeneous pool is introduced into a population of cells that do not exhibit the desired biological function. Subpopulations of the cells are then screened for those which exhibit a predetermined biological function. From that subpopulation, oligonucleotides capable of carrying out the desired biological function are isolated. U.S. Pat. Nos. 5,763,192, 5,814,476, 5,723,323, and 5,817,483 describe processes for producing peptides or polypeptides. This is done by producing stochastic genes or fragments thereof, and then introducing these genes into host cells which produce one or more proteins encoded by the stochastic genes. The host cells are then screened to identify those clones producing peptides or polypeptides having the desired activity.

Another method for producing peptides or polypeptides is described in PCT/US98/20094 (WO99/15650) filed by Athersys, Inc. Known as “Random Activation of Gene Expression for Gene Discovery” (RAGE-GD), the process involves the activation of endogenous gene expression or over-expression of a gene by in situ recombination methods. For example, expression of an endogenous gene is activated or increased by integrating a regulatory sequence into the target cell which is capable of activating expression of the gene by non-homologous or illegitimate recombination. The target DNA is first subjected to radiation, and a genetic promoter inserted. The promoter eventually locates a break at the front of a gene, initiating transcription of the gene. This results in expression of the desired peptide or polypeptide.

It will be appreciated that these methods can also be used to create comprehensive IL-17 like protein expression libraries, which can subsequently be used for high throughput phenotypic screening in a variety of assays, such as biochemical assays, cellular assays, and whole organism assays (e.g., plant, mouse, etc.).

Chemical Derivatives Chemically modified derivatives of the TNFr/OPG-like polypeptides may be prepared by one skilled in the art, given the disclosures set forth hereinbelow. TNFr/OPG-like polypeptide derivatives are modified in a manner that is different, either in the type or location of the molecules naturally attached to the polypeptide. Derivatives may include molecules formed by the deletion of one or more naturally-attached chemical groups. The polypeptide comprising the amino acid sequence of SEQ ID NOS: 2 or 4, or an TNFr/OPG-like polypeptide variant, may be modified by the covalent attachment of one or more polymers. For example, the polymer selected is typically water soluble so that the protein to which it is attached does not precipitate in an aqueous environment, such as a physiological environment. Included within the scope of suitable polymers is a mixture of polymers. Preferably, for therapeutic use of the end-product preparation, the polymer will be pharmaceutically acceptable. The polymers each may be of any molecular weight and may be branched or unbranched. The polymers each typically have an average molecular weight of between about 2 kDa to about 100 kDa (the term “about” “about” indicating that in preparations of a water soluble polymer, some molecules will weigh more, some less, than the stated molecular weight). The average molecular weight of each polymer is preferablyis between about 5 kDa and 5 kDa, about 50 kDa, more preferably between about 12 kDa andto about 40 kDa and most preferably between about 20 kDa andto about 35 kDa.

Suitable water soluble polymers or mixtures thereof include, but are not limited to, N-linked or O-linked carbohydrates, sugars, phosphates,carbohydrates; sugars; phosphates; polyethylene glycol (PEG) (including the forms of PEG that have been used to derivatize proteins, including mono-(C1-C10) alkoxy- or aryloxy-polyethylene glycol),glycol); monomethoxy-polyethylene glycol,glycol; dextran (such as low molecular weight dextran, of, for example about 6 kD), cellulose, or otherdextran of, for example, about 6 kDa);, cellulose; or carbohydrate basedother carbohydrate-based polymers, poly-(N-vinyl pyrrolidone) polyethylene glycol, propylene glycol homopolymers, a polypropylene oxide/ethylene oxide co-polymer, polyoxyethylated polyols (e.g., glycerol) and polyvinyl alcohol. Also encompassed by the present invention are bifunctional crosslinking molecules which may be used to prepare covalently attached multimers of the polypeptide comprising the amino acid sequence of SEQ ID NOS: 2 or 4 or an TNFr/OPG-like polypeptide variant.

In general, chemical derivatization may be performed under any suitable condition used to react a protein with an activated polymer molecule. Methods for preparing chemical derivatives of polypeptides will generally comprise the steps of (a) reacting the polypeptide with the activated polymer molecule (such as a reactive ester or aldehyde derivative of the polymer molecule) under conditions whereby the polypeptide comprising the amino acid sequence of SEQ ID NOS: 2 or 4, or an TNFr/OPG-like polypeptide variant becomes attached to one or more polymer molecules, and (b) obtaining the reaction product(s). The optimal reaction conditions will be determined based on known parameters and the desired result. For example, the larger the ratio of polymer molecules:protein, the greater the percentage of attached polymer molecule. In one embodiment, the TNFr/OPG-like polypeptide derivative may have a single polymer molecule moiety at the amino terminus. See,terminus (see, for example, U.S. Pat. No. 5,234,784).

The pegylation of the polypeptide may be specifically may be carried out by any of the pegylation reactions known in the art, as described for example in the following references: Francis et al., Focus on Growth Factors, 3:4-10 (1992); EP 0154316; EP 0401384 and U.S. Pat. No. 4,179,337. For example, pegylation may be carried out via an acylation reaction or an alkylation reaction with a reactive polyethylene glycol molecule (or an analogous reactive water-soluble polymer) as described herein. For the acylation reactions, the polymer(s) selected should have a single reactive ester group. For reductive alkylation, the polymer(s) selected should have a single reactive aldehyde group. A reactive aldehyde is, for example, polyethylene glycol propionaldehyde, which is water stable, or mono C1-C10 alkoxy or aryloxy derivatives thereof (see U.S. Pat. No. 5,252,714).

In another embodiment, TNFr/OPG-like polypeptides may be chemically coupled to biotin, and the biotin/TNFr/OPG-like polypeptide molecules which are conjugated are then allowed to bind to avidin, resulting in tetravalent avidin/biotin/TNFr/OPG-like polypeptide molecules. TNFr/OPG-like polypeptides may also be covalently coupled to dinitrophenol (DNP) or trinitrophenol (TNP) and the resulting conjugates precipitated with anti-DNP or anti-TNP-IgM to form decameric conjugates with a valency of 10.

Generally, conditions which may be alleviated or modulated by the administration of the present TNFr/OPG-like polypeptide derivatives include those described herein for TNFr/OPG-like polypeptides. However, the AGP-TNFr/OPG-like polypeptide derivatives disclosed herein may have additional activities, enhanced or reduced biological activity, or other characteristics, such as increased or decreased half-life, as compared to the non-derivatized molecules.

Microarray

It will be appreciated that DNA microarray technology can be utilized in accordance with the present invention. DNA microarrays are miniature, high density arrays of nucleic acids positioned on a solid support, such as glass. Each cell or element within the array has numerous copies of a single species of DNA which acts as a target for hybridization for its cognate mRNA. In expression profiling using DNA microarray technology, mRNA is first extracted from a cell or tissue sample and then converted enzymatically to fluorescently labeled cDNA. This material is hybridized to the microarray and unbound cDNA is removed by washing. The expression of discrete genes represented on the array is then visualized by quantitating the amount of labeled cDNA which is specifically bound to each target DNA. In this way, the expression of thousands of genes can be quantitated in a high throughput, parallel manner from a single sample of biological material.

This high throughput expression profiling has a broad range of applications with respect to the TNFr/OGP-like molecules of the invention, including, but not limited to: the identification and validation of TNFr/OGP-like disease-related genes as targets for therapeutics; molecular toxicology of TNFr/OGP-like molecules and inhibitors thereof; stratification of populations and generation of surrogate markers for clinical trials; and the enhancement of TNFr/OGP-like related small molecule drug discovery by aiding in the identification of selective compounds in high throughput screens (HTS).

Selective Binding Agents

As used herein, the term “selective binding agent” reffers to a molecule which has specificity for one or more TNFr/OGP-like polypeptides. Suitable selective binding agents include antibodies and dervatives thereof, polypeptides, and samll molecules. Suitable selective binding agents may be prepared using methods known in the art. An exemplary TNFr/OGP-like polypeptide selective binding agent of the present invention is capable of binding a certain portion of the TNFr/OGP-like polypeptide thereby inhibiting the binding of the polypeptide to the TNFr/OGP-like receptor(s).

Selective binding agents such as antibodies and antibody fragemtns that bind TNFr/OGP-like are within the scope of the present invention. The antibodies may be may be polyclonal including monospecific polyclonal, monoclonal (mAbs), recombinant, chimeric, humanized (such as CDR-grafted), human, single chain, and/or bispecific, as well as fragements, variants, or derivatives thereof. Antibody fragments include those portions of the antibody which bind to an epitope on the TNFr/OPG-like polypeptide. Examples of such fragments include Fab and F(ab′) fragments generated by enzymatic cleavage of full-length antibodies. Other binding fragments include those generated by recombinant DNA techniques, such as the expression of recombinant plasmids containing nucleic acid sequences encoding antibody variable regions.

Polyclonal antibodies directed toward a TNFr/OPG-like polypeptide generally are produced in animals (e.g., rabbits or mice) by means of multiple subcutaneous or intraperitoneal injections of TNFr/OPG-like polypeptide and an adjuvant. It may be useful to conjugate a TNFr/OPG-like polypeptide, or a variant, fragment, or derivative thereof to a carrier protein that is immunogenic in the species to be immunized, such as keyhole limpet heocyanin, serum, albumin, bovine thyroglobulin, or soybean trypsin inhibitor. Also, aggregating agents such as alum are used to enhance the immune response. After immunization, the animals are bled and the serum is assayed for anti-TNFr/OPG-like antibody titer.

Monoclonal antibodies directed toward TNFr/OPG-like polypeptides are produced using any method which provides for the production of antibody molecules by continuous cell lines in culture. Examples of suitable methods for preparing monoclonal antibodies include the hybridoma methods of Kohler et al., Nature, 256:495-497 (1975) and the human B-cell hybridoma method, Kozbor, J. Immunol., 133:3001 (1984); Brodeur et al., Monoclonal Antibody Production Techniques and Applications, pp. 51-63 (Marcel Dekker, Inc., New York, 1987). Also provided by the invention are hybridoma cell lines which produce monoclonal antibodies reactive with TNFr/OPG-like polypeptides.

Monoclonal antibodies of the invention may be modified for use as therapeutics. One embodiment is a “chimeric” antibody in which a portion of the heavy and/or light chain is identical with or homologous to a corresponding sequence in antibodies derived from a particular species or belonging to a particular antibody class or subclass, while the remainder of the chain(s) is identical with or homologous to a corresponding sequence in antibodies derived from another species or belonging to another antibody class or subclass. Also included are fragments of such antibodies, so long as they exhibit the desired biological activity. See, U.S. Pat. No. 4,816,567; Morrison et al., Proc. Natl. Acad. Sci., 81: 6851-6855 (1985).

In another embodiment, a monoclonal antibody of the invention is a “humanized” antibody. Methods for humanizing non-human antibodies are well known in the art. See U.S. Pat. Nos. 5,585,089 and 5,693,762. Generally, a humanized antibody has one or more amino acid residues introduced into it from a source which is non-human. Humanization can be performed, for example, using methods known in the art (Jones et al., Nature 321: 522-525 (1986); Riechmann et al., Nature, 332: 323-327 (1988); Verhoeyen et al., Science 239:1534-1536 (1988)), by substituting at least a portion of a rodent complementarity-determining region (CDR) for the corresponding regions of a human antibody.

Also encompassed by the invention are human antibodies which bind TNFr/OPG-like polypeptides. Using transgentic animals (e.g.) mice that are capable of producing a repertoire of human antibodies in the absence of endogenous immunoglobin production such antibodies are produced by immunization with a TNFr/OPG-like antigen (i.e., having at least 6 contiguous amino acids), optionally conjugated to a carrier. See for example, Jakobovits et al., Proc. Natl. Acad. Sci., 90: 2551-2555 (1993); Jakobovits et al., Nature 362: 255-258 (1993); Bruggermann et al., Year in Immuno., 7:33 (1993). In one method, such transgenic animals are produced by incapacitating the endogenous loci encoding the heavy and light immunoglobulin chains therein, and inserting loci encoding human heavy and light chain proteins into the genome thereof. Partially modified animals, that is those having less than the full complement of modifications, are then cross-bred to obtain an animal having all of the desired immune system modifications. When administered an immunogen, these transgenic animals produce antibodies with human variable regions, including human (rather than e.g., murine) amino acid sequences, including variable regions which are immunospecific for these antigens. See PCT application nos. PCT/US96/05928 and PCT/US93/06926. Additional methods are described in U.S. Pat. No. 5,545,807, PCT application nos. PCT/US91/245, PCT/GB89/01207, and in EP 546073B1 and EP 546073B1.

Human antibodies may also be produced by the expression of recombinant DNA in host cells or by expression in hybridoma cells as described herein.

In an alternate emboidment, human antibdoies can be produced from phage-display libraries (Hoogenboom et al., J. Mol. Biol. 227: 381 (1991); Marks et al., J. Mol. Biol. 222:581 (1991). These processes mimic immune selection through the display of antibody repertoires on the surface of filamentous bacteriophage, and subsequent selection of phage by their binding to an antigen of choice. One such technique is described in PCT Application no. PCT/US98/17364, which describes the isolation of high affinity and functional agonistic antibodies for MPL- and msk- receptors using such an approach.

Chimeric, CDR grafted, and humanized antibodies are typically produced by recombinant methods. Nucleic acids encoding the antibodies are introduced into host cells and expressed using materials and procedures described herein. In a preferred embodiment, the antibodies are produced in mammalian host cells, such as CHO cells. Monoclonal (e.g. human) antibodies may be produced by the expression of recombinant DNA in host cells or by expression in hybridoma cells as described herein.

The anti-TNFr/OGP-like antibodies of the invention may be employed in any known assay method, such as competitive binding assays, direct and indirect sandwich assays, and immunoprecipitation assays (Sola, Monoclonal Antibodies: A Manual of Techniques, pp. 147-158 (CRC Press, Inc., 1987)) for the detection and quantitation of TNFr/OPG-like polypeptides. The antibodies will bind TNFr/OGP-like polypeptides with an affinity which is appropriate for the assay method being employed.

For diagnostic applications, in certain embodiments, anti-TNFr/OPG-like antibodies typically will be labeled with a detectable moiety. The detectable moiety can be any one which is capable of producing, either directly or indirectly, a detectable signal. For example, the detectable moiety may be a radioisotope, such as ³H, ¹⁴C, ³²P, ³⁵S, or ¹²⁵I a fluorescent or chemiluminescent compound, such as fluorescein isothiocyanate, rhodamine, or luciferin; or an enzyme, such as alkaline phosphatase, β-galactosidase, or horseradish peroxidase. Bayer et al., Meth. Enz., 184: 138-163 (1990

Competitive binding assays rely on the ability of a labeled standard (e.g., a TNFr/OPG-like polypeptide, or an immunologically reactive portion thereof) to compete with the test sample analyte (a TNFr/OPG-like polypeptide) for binding with a limited amount of anti TNFr/OPG-like antibody. The amount of a TNFr/OPG-like polypeptide in the test sample is inversely proportional to the amount of standard that becomes bound to the antibodies. To facilitate determining the amount of standard that becomes bound, the antibodies typically are insolubilized before or after the competition, so that the standard and analyte that are bound to the antibodies may conveniently be separated from the standard and analyte which remain unbound.

Sandwich assays typically involve the use of two antibodies, each capable of binding to a different immunogenic portion, or epitope, of the protein to be detected and/or quantitated. In a sandwich assay, the test sample analyte is typically bound by a first antibody which is immobilized on a solid support, and thereafter a second antibody binds to the analyte, thus forming an insoluble three part complex. See, e.g., U.S. Pat. No. 4,376,110. The second antibody may itself be labeled with a detectable moiety (direct sandwich assays) or may be measured using an anti-immunoglobulin antibody that is labeled with a detectable moiety (indirect sandwich assays). For example, one type of sandwich assay is an enzyme-linked immunosorbent assay (ELISA), in which case the detectable moiety is an enzyme.

The selective binding agents of the invention, including TNFr/OGP-like antibodies, may be used as therapeutics. These therapeutic agents are generally agonists or antagonists, in that they either enhance or reduce, respectively, at least one of the biological activities of a TNFr/OPG-like polypeptide. In one embodiment, antagonist antibodies of the invention are antibodies or binding fragments thereof which are capable of specifically binding to a TNFr/OPG-like polypeptide and which are capable of inhibiting or eliminating the functional activity of a TNFr/OPG-like polypeptide in vivo or in vitro. In preferred embodiments, the selctive binidng agent e.g., an antagonist antibody will inhibit the functional activity of a TNFr/OPG-like polypeptide by at least about 50%, and preferably by at least about 80%. In another embodiment, the selective binding agent may be an antibody that is capable of interacting with a TNFr/OPG-like binding partner (a ligand or receptor) thereby inhibiting or eliminating TNFr/OPG-like activity in vitro or in vivo. Selective binding agents, including agonist and antagonist anti-TNFr/OPG-like antibodies are identified by screening assays which are well known in the art.

The anti-TNFr/OPG-like antibodies of the invention also are useful for in vivo imaging. An antibody labeled with a detectable moiety may be administered to an animal, preferably into the bloodstream, and the presence and location of the labeled antibody in the host is assayed. The antibody may be labeled with any moiety that is detectable in an animal, whether by nuclear magnetic resonance, radiology, or other detection means known in the art.

The invention also relates to a kit comprising TNFr/OPG-like selective binding agents (such as antibodies) and other reagents useful for detecting TNFr/OPG-like polypeptide levels in biological samples. Such reagents may include a secondary activity, a detectable label, blocking serum, positive and negative control samples, and detection reagents.

As noted, the TNFr/OPG-like receptor(s) recited herein are useful for identifying or developing novel agonists and antagonists of the TNFr/OPG-like signaling pathway. Such agonists and antagonists include soluble TNFr/OPG-like receptor(s), anti-TNFr/OPG-like receptor antibodies, small molecules, or antisense oligonucleotides, and they may also be used for treating one or more of the diseases/disorders described herein.

Additional Agonist and Antagonist Molecules

As defined herein, agonist or antagonist molecules either enhance or reduce, respectively, at least one of the biological activities of a TNFr/OPG-like polypeptide. Antagonists are capable of interacting with the TNFr/OPG-like receptor itself and/or with a TNFr/OPG-like binding partner (such as a ligand or receptor), thereby inhibiting or eliminating TNFr/OPG-like polypeptide activity in vitro or in vivo. Agonists are those molecules that can specifically bind to the TNFr/OPG-like molecule and function like their native ligands to activate the receptor. Agonists can also interact with a TNFr/OPG-like binding partner (such as a ligand) to enhance its binding to the TNFr/OPG-like polypeptides, thereby enhancing the biological activity of the TNFr/OPG-like molecule. It will be appreciated that the agonists and antagonists described herein are not limited to selective binding agents. In addition to selective binding agents, other suitable agonist and antagonist molecules include, but are not limited to, soluble TNFr/OPG-like polypeptides, small molecules, and antisense oligonucleotides, any of which can be used for treating one or more disease or disorder, including those described herein.

TNFr/OPG-like polypeptides can be used to clone TNFr/OPG-like ligand(s) using an “expression cloning” strategy. Radiolabeled (125-Iodine) TNFr/OPG-like polypeptide or “affinity/activity-tagged” TNFr/OPG-like polypeptide (such as an Fc fusion or an alkaline phosphatase fusion) can be used in binding assays to identify a cell type or a cell line or tissue that expresses TNFr/OPG-like ligand(s). RNA isolated from such cells or tissues can then be converted to cDNA, cloned into a mammalian expression vector, and transfected into mammalian cells (for example, COS, or 293) to create an expression library. Radiolabeled or tagged TNFr/OPG-like polypeptide can then be used as an affinity reagent to identify and isolate the subset of cells in this library expressing TNFr/OPG-like ligand(s). DNA is then isolated from these cells and transfected into mammalian cells to create a secondary expression library in which the fraction of cells expressing TNFr/OPG-like ligand(s) would be many-fold higher than in the original library. This enrichment process can be repeated iteratively until a single recombinant clone containing a TNFr/OPG-like ligand is isolated. isolation of TNFr/OPG-like ligand(s) is useful for identifying or developing novel agonists and antagonists of the TNFr/OPG-like signaling pathway. Such agonists and antagonists include TNFr/OPG-like ligand(s), anti-TNFr/OPG-like ligand antibodies, small molecules or antisense oligonucleotides.

Genetically Engineered Non-Human Animals

Additionally included within the scope of the present invention are non-human animals such as mice, rats, or other rodents, rabbits, goats, or sheep, or other farm animals, in which the gene (or genes) encoding a native TNFr/OPG-like polypeptide has (have) been disrupted (“knocked out”) such that the level of expression of this gene or genes is (are) significantly decreased or completely abolished. Such animals may be prepared using techniques and methods such as those described in U.S. Pat. No. 5,557,032.

The present invention further includes non-human animals such as mice, rats, or other rodents, rabbits, goats, or sheep, or other farm animals, in which either the native form of the TNFr/OPG-like polypeptide gene(s) for that animal or a heterologous TNFr/OPG-like polypeptide gene(s) is (are) over expressed by the animal, thereby creating a “transgenic” animal. Such transgenic animals may be prepared using well known methods such as those described in U.S. Pat. No 5,489,743 and PCT application No. WO94/28122.

The present invention further includes non-human animals in which the promoter for one or more of the TNFr/OPG-like polypeptides of the present invention is either activated or inactivated (e.g., by using homologous recombination methods) to alter the level of expression of one or more of the native TNFr/OPG-like polypeptides.

These non-human animals may be used for drug candidate screening. In such screening, the impact of a drug candidate on the animal may be measured. For example, drug candidates may decrease or increase the expression of the TNFr/OPG-like polypeptide gene. In certain embodiments, the amount of TNFr/OPG-like polypeptide, or a fragment(s), that is produced may be measured after the exposure of the animal to the drug candidate. Additionally, in certain embodiments, one may detect the actual impact of the drug candidate on the animal. For example, the overexpression of a particular gene may result in, or be associated with, a disease or pathological condition. In such cases, one may test a drug candidate's ability to decrease expression of the gene or its ability to prevent or inhibit a pathological condition. In other examples, the production of a particular metabolic product such as a fragment of a polypeptide, may result in, or be associated with, a disease or pathological condition. In such cases, one may test a drug candidate's ability to decrease the production of such a metabolic product or its ability to prevent or inhibit a pathological condition.

Assaying for Other Modulators of TNFr/OPG-Like Polypeptide Activity

In some situations, it may be desirable to identify molecules that are modulators, i.e., agonists or antagonists, of the activity of TNFr/OPG-like polypeptide. Natural or synthetic molecules that modulate TNFr/OPG-like polypeptides may be identified using one or more screening assays, such as those described herein. Such molecules may be administered either in an ex vivo manner, or in an in vivo manner by injection, or by oral delivery, implantation device, or the like.

“Test molecule(s)” refers to the molecules() that is/are under evaluation for the ability to modulate (i.e., increase or decrease) the activity of a TNFr/OPG-like polypeptide. Most commonly, a test molecule will interact directly with a TNFr/OPG-like polypeptide. However, it is also contemplated that a test molecule may also modulate TNFr/OPG-like polypeptide activity indirectly, such as by affecting TNFr/OPG-like gene expression, or by binding to a TNFr/OPG-like binding partner (e.g., receptor or ligand). In one embodiment, a test molecule will bind to a TNFr/OPG-like polypeptide with an affinity constant of at least about 10⁻⁶ M, preferably about 10⁻⁸ M, more preferably about 10⁻⁹ M, and even more preferably about 10⁻¹⁰ M.

Methods for identifying compounds which interact with TNFr/OPG-like polypeptides are encompassed by the present invention. In certain embodiments, a TNFr/OPG-like polypeptide is incubated with a test molecule under conditions which permit the interaction of the test molecule with a TNFr/OPG-like polypeptide, and the extent of the interaction can be measured. The test molecule(s) can be screened in a substantially purified form or in a crude mixture. Test molecule(s) can be nucleic acid molecules, proteins, peptides, carbohydrates, lipids, or small molecular weight organic or inorganic compounds. Once a set of test molecules has been identified as interacting with a TNFr/OPG-like polypeptide, the molecules may be further evaluated for their ability to increase or decrease TNFr/OPG-like polypeptide activity.

The measurement of the interaction of test molecules with TNFr/OPG-like polypeptides may be carried out in several formats, including cell-based binding assays, membrane binding assays, solution-phase assays and immunoassays. In general, test molecules are incubated with a TNFr/OPG-like polypeptide for a specified period of time, and TNFr/OPG-like polypeptide activity is determined by one or more assays described herein for measuring biological activity.

The interaction of test molecules with TNFr/OPG-like polypeptides may also be assayed directly using polyclonal or monoclonal antibodies in an immunoassay. Alternatively, modified forms of TNFr/OPG-like polypeptides containing epitope tags as described herein may be used in solution and immunoassays.

In certain embodiments, a TNFr/OPG-like polypeptide agonist or antagonist may be a protein, peptide, carbohydrate, lipid, or small molecular weight molecule which interacts with TNFr/OPG-like polypeptide to regulate its activity. Potential protein antagonists of TNFr/OPG-like polypeptide include antibodies which interact with active regions of the polypeptide and inhibit or eliminate at least one activity of TNFr/OPG-like molecules. Molecules which regulate TNFr/OPG-like polypeptide expression include nucleic acids which are complementary to nucleic acids encoding a TNFr/OPG-like polypeptide, or are complementary to nucleic acids sequences which direct or control the expression of TNFr/OPG-like polypeptide, and which act as anti-sense regulators of expression.

In the event that TNFr/OPG-like polypeptides display biological activity through an interaction with a binding partner (e.g., a ligand), a variety of in vitro assays may be used to measure the binding of a TNFr/OPG-like polypeptide to the corresponding binding partner (such as a selective binding agent or ligand). These assays may be used to screen test molecules for their ability to increase or decrease the rate and/or the extent of binding of a TNFr/OPG-like polypeptide to its binding partner. In one assay, a TNFr/OPG-like polypeptide is immobilized in the wells of a microtiter plate. Radiolabeled TNFr/OPG-like binding partner (for example, iodinated TNFr/OPG-like binding partner) and the test molecule(s) can then be added either one at a time (in either order) or simultaneously to the wells. After incubation, the wells can be washed and counted, using a scintillation counter, for radioactivity to determine the extent to which the binding partner bound to TNFr/OPG-like polypeptide. Typically, the molecules will be tested over a range of concentrations, and a series of control wells lacking one or more elements of the test assays can be used for accuracy in the evaluation of the results. An alternative to this method involves reversing the “positions” of the polypeptides, i.e., immobilizing TNFr/OPG-like binding partner to the microtiter plate wells, incubating with the test molecule and radiolabeled TNFr/OPG-like polypeptide, and determining the extent of TNFr/OPG-like polypeptide binding. See, for example, chapter 18, Current Protocols in Molecular Biology, Ausubel et al., eds., John Wiley & Sons, New York, N.Y. (1995).

As an alternative to radiolabelling, a TNFr/OPG-like polypeptide or its binding partner may be conjugated to biotin and the presence of biotinylated protein can then be detected using streptavidin linked to an enzyme, such as horseradish peroxidase (HRP) or alkaline phosphatase (AP), that can be detected colorometrically, or by fluorescent tagging of streptavidin. An antibody directed to a TNFr/OPG-like polypeptide or to a TNFr/OPG-like binding partner and conjugated to biotin may also be used and can be detected after incubation with enzyme-linked streptavidin linked to AP or HRP.

A TNFr/OPG-like polypeptide and a TNFr/OPG-like binding partner can also be immobilized by attachment to agarose beads, acrylic beads or other types of such inert solid phase substrates. The substrate-protein complex can be placed in a solution containing the complementary protein and the test compound. After incubation, the beads can be precipitated by centrifugation, and the amount of binding between a TNFr/OPG-like polypeptide and its binding partner can be assessed using the methods described herein. Alternatively, the substrate-protein complex can be immobilized in a column, and the test molecule and complementary protein are passed through the column. The formation of a complex between a TNFr/OPG-like polypeptide and its binding partner can then be assessed using any of the techniques set forth herein, i.e., radiolabelling, antibody binding, or the like.

Another in vitro assay that is useful for identifying a test molecule which increases or decreases the formation of a complex between a TNFr/OPG-like binding polypeptide and a TNFr/OPG-like binding partner is a surface plasmon resonance detector system such as the BIAcore assay system (Pharmacia, Piscataway, N.J.). The BIAcore system may be carried out using the manufacturer's protocol. This assay essentially involves the covalent binding of either TNFr/OPG-like polypeptide or a TNFr/OPG-like binding partner to a dextran-coated sensor chip which is located in a detector. The test compound and the other complementary protein can then be injected, either simultaneously or sequentially, into the chamber containing the sensor chip. The amount of complementary protein that binds can be assessed based on the change in molecular mass which is physically associated with the dextran-coated side of the sensor chip; the change in molecular mass can be measured by the detector system.

In some cases, it may be desirable to evaluate two or more test compounds together for their ability to increase or decrease the formation of a complex between a TNFr/OPG-like polypeptide and a TNFr/OPG-like binding partner. In these cases, the assays set forth herein can be readily modified by adding such additional test compound(s) either simultaneous with, or subsequent to, the first test compound. The remainder of the steps in the assay are as set forth herein.

In vitro assays such as those described herein may be used advantageously to screen large numbers of compounds for effects on complex formation by TNFr/OPG-like polypeptide and TNFr/OPG-like binding partner. The assays may be automated to screen compounds generated in phage display, synthetic peptide, and chemical synthesis libraries.

Compounds which increase or decrease the formation of a complex between a TNFr/OPG-like polypeptide and a TNFr/OPG-like binding partner may also be screened in cell culture using cells and cell lines expressing either TNFr/OPG-like polypeptide or TNFr/OPG-like binding partner. Cells and cell lines may be obtained from any mammal, but preferably will be from human or other primate, canine, or rodent sources. The binding of a TNFr/OPG-like polypeptide to cells expressing TNFr/OPG-like binding partner at the surface is evaluated in the presence or absence of test molecules, and the extent of binding may be determined by, for example, flow cytometry using a biotinylated antibody to a TNFr/OPG-like binding partner. Cell culture assays can be used advantageously to further evaluate compounds that score positive in protein binding assays described herein.

Cell cultures can also be used to screen the impact of a drug candidate. For example, drug candidates may decrease or increase the expression of the TNFr/OPG-like polypeptide gene. In certain embodiments, the amount of TNFr/OPG-like polypeptide or a fragment(s) that is produced may be measured after exposure of the cell culture to the drug candidate. In certain embodiments, one may detect the actual impact of the drug candidate on the cell culture. For example, the overexpression of a particular gene may have a particular impact on the cell culture. In such cases, one may test a drug candidate's ability to increase or decrease the expression of the gene or its ability to prevent or inhibit a particular impact on the cell culture. In other examples, the production of a particular metabolic product such as a fragment of a polypeptide, may result in, or be associated with, a disease or pathological condition. In such cases, one may test a drug candidate's ability to decrease the production of such a metabolic product in a cell culture.

A yeast two-hybrid system (Chien et al., Proc. Natl. Acad. Sci. USA, 88:9578-9583, 1991) can be used to identify novel polypeptides that bind to, or interact with, TNFr/OPG-like polypeptides. As an example, a yeast-two hybrid bait construct can be generated in a vector (such as the pAS2-1 from Clontech) which encodes a yeast GAL4-DNA binding domain fused to the TNFr/OPG-like polynucleotide. This bait construct may be used to screen human cDNA libraries wherein the cDNA library sequences are fused to GAL4 activation domains. Positive interactions will result in the activation of a reporter gene such as -Gal. Positive clones emerging from the screening may be characterized further to identify interacting proteins.

P38 Inhibitors

A new approach to intervention between the extracellular stimulus and the secretion of IL-1 and TNF from the cell involves blocking signal transduction through inhibition of a kinase which lies on the signal pathway. One example is through inhibition of P-38 (also called “RK” or “SAPK-2”, Lee et al., Nature, 372:739 (1994)), a known ser/thr kinase (clone reported in Han et al., Biochimica Biophysica Acta, 1265:224-227 (1995)). A linear relationship has been shown for effectiveness in a competitive binding assay to P-38, and the same inhibitor diminishing the levels of IL-1 secretion from monocytes following LPS stimulation. Following LPS stimulation of monocytes, the levels of messenger RNA for TNF-a have been shown to increase 100 fold, but the protein levels of TNF-a are increased 10,000 fold. Thus, a considerable amplification of the TNF signaling occurs at the translational level. Following LPS stimulation of monocytes in the presence of a P-38 inhibitor, the levels of mRNA are not affected, but the levels of final TNF protein are dramatically reduced (up to 80-90% depending on the effectiveness of the P-38 inhibitor). Thus, the above experiments lend strong support to the conclusion that inhibition of P-38 leads to diminished translational efficiency. Further evidence that TNF is under translational control is found in the deletion experiments of Beutler et al. and Lee, wherein segments of 3′ untranslated mRNA (3′ UTR) are removed resulting in high translational efficiency for TNF. More importantly, the P-38 inhibitors did not have an effect on the level of TNF (i.e., translational efficiency) when the appropriate segments of TNF mRNA are deleted. Thus, the correlative data between the level of binding of inhibitors to P-38 and the diminished IL-1 and TNF levels following LPS stimulation with the same inhibitors, plus the above biochemical evidence regarding the effect of P-38 inhibitors on translational efficiency of both TNF and IL-1 make a strong cause and effect relationship. The role of P-38 in the cell is still being delineated; so therefore, other beneficial effects regarding inflammatory diseases or other disease states obtained from its inhibition maybe forthcoming.

Elevated levels of TNF and/or IL-1 may contribute to the onset, etiology, or exacerbate a number of disease states, including, but not limited to: rheumatoid arthritis; osteoarthritis; rheumatoid spondylitis; gouty arthritis; inflammatory bowel disease; adult respiratory distress syndrome (ARDS); psoriasis; Crohn's disease; allergic rhinitis; ulcerative colitis; anaphylaxis; contact dermatitis; asthma; antiviral therapy including those viruses sensitive to TNF inhibition—HIV-1, HIV-2, HIV-3, cytomegalovirus (CMV), influenza, adenovirus, and the herpes viruses including HSV-1, HSV-2, and herpes zoster; muscle degeneration; cachexia; Reiter's syndrome; type II diabetes; bone resorption diseases; graft vs. host reaction; ischemia reperfusion injury; atherosclerosis; brain trauma; Alzheimer's disease; multiple sclerosis; cerebral malaria; sepsis; septic shock; toxic shock syndrome; fever and mylagias due to infection.

Substituted imidazole, pyrrole, pyridine, pyrimidine and the like compounds have been described for use in the treatment of cytokine mediated diseases by inhibition of proinflammatory cytokines, such as IL-1, IL-6, IL-8 and TNF. Substituted imidazoles for use in the treatment of cytokine mediated diseases have been described in U.S. Pat. No. 5,593,992; WO 93/14081; WO 97/18626; WO 96/21452; WO 96/21654; WO 96/40143; WO 97/05878; WO 97/05878; (each of which is incorporated herein by reference in its entirety). Substituted imidazoles for use in the treatment of inflammation has been described in U.S. Pat. No. 3,929,807 (which is incorporated herein by reference in its entirety). Substituted pyrrole compounds for use in the treatment of cytokine mediated diseases have been described in WO 97/05877; WO 97/05878; WO 97/16426; WO 97/16441; and WO 97/16442 (each of which is incorporated herein by reference in its entirety). Substituted aryl and heteroaryl fused pyrrole compounds for use in the treatment of cytokine mediated diseases have been described in WO 98/22457 (which is incorporated herein by reference in its entirety). Substituted pyridine, pyrimidine, pyrimidinone and pyridazine compounds for use in the treatment of cytokine mediated diseases have been described in WO 98/24780; WO 98/24782; WO 99/24404; and WO 99/32448 (each of which is incorporated herein by reference in its entirety).

Internalizing Proteins The tat protein sequence (from HIV) can be used to internalize proteins into a cell. See e.g., Falwell et al., Proc. Natl. Acad. Sci. USA, 91:664-668 (1994). For example, an 11 amino acid sequence (YGRKKRRQRRR: SEQ ID NO: 21) of the HIV tat protein (termed the “protein “protein transduction domain”,domain”, or TAT PDT) has been described as mediating delivery across the cytoplasmic membrane and the nuclear membrane of a cell. See Schwarze et al., Science, 285:569-285:1569-1572 (1999); and Nagahara et al., Nature Medicine, 4:1449-1452 (1998). In these procedures, FITC-constructs (FITC-GGGGYGRKKRRQRRR; SEQ ID NO: 22) are prepared which bind to cells as observed by fluorescence-fluorescence-activated cell sorting (FACS) analysis, and these constructs penetrate tissues after i.p. adminstration. Next, tat-bgal fusion proteins are constructed. Cells treated with this construct demonstrated b-gal activity. Following injection, a number of tissues, including liver, kidney, lung, heart, and brain tissue, have been found to demonstrate expression using these procedures. It is believed that these constructions underwent some degree of unfolding in order to enter the cell; as such, refolding may be required after entering the cell.

It will thus be appreciated that the tat protein sequence may be used to internalize a desired protein or polypeptide into a cell. For example, using the tat protein sequence, an TNFr/OPG-like antagonist (such as an anti-TNFr/OPG-like selective binding agent, small molecule, soluble receptor, or antisense oligonucleotide) can be administered intracellularly to inhibit the activity of an TNFr/OPG-like molecule. As used herein, the term “TNFr/OPG-like molecule” refers to both TNFr/OPG-like nucleic acid molecules and TNFr/OPG-like polypeptides as defined herein. Where desired, the TNFr/OPG-like protein itself may also be internally administered to a cell using these procedures. See also, Strauss, E., “Introducing Proteins Into the Body's Cells”, Science, 285:1466-1467 (1999).

Cell Source Identification using TNFr/OPG-Like Polypeptides

In accordance with certain embodiments of the invention, it may be useful to be able to determine the source of a certain cell type associated with a TNFr/OPG-like polypeptide. For example, it may be useful to determine the origin of a disease or pathological condition as an aid in selecting an appropriate therapy. In other embodiments, one may use the TNFr/OPG-like polypeptide to make antibodies that are specific for TNFr/OPG-like polypeptide.

Therapeutic Uses

Bone tissue provides support for the body and consists of mineral (largely calcium and phosphorous), a matrix of collagenous and noncollagenous proteins, and cells. Three types of cells found in bone, osteocytes, osteoblasts, and osteoclasts, are involved in the dynamic process by which bone is continually formed and resorbed. Osteoblasts promote formation of bone tissue whereas osteoclasts are associated with resorption. Resorption, or the dissolution of bone matrix and mineral, is a fast and efficient process compared to bone formation and can release large amounts of mineral from bone. Osteoclasts are involved in the regulation of the normal remodeling of skeletal tissue and in resorption induced by hormones. For instance, resorption is stimulated by the secretion of parathyroid hormone in response to decreasing concentrations of calcium ion in extracellular fluids. In contrast, inhibition of resorption is the principal function of calcitonin. In addition, metabolites of vitamin D alter the responsiveness of bone to parathyroid hormone and calcitonin.

Following skeletal maturity, the amount of bone in the skeleton reflects the balance (or imbalance) of bone formation and bone resorption. Peak bone mass occurs after skeletal maturity prior to the fourth decade. Between the fourth and fifth decades, the equilibrium shifts and bone resorption dominates. The inevitable decrease in bone mass with advancing years starts earlier in females than males and is distinctly accelerated after menopause in some females (principally those of Caucasian and Asian descent).

Osteopenia is a condition relating generally to any decrease in bone mass to below normal levels. Such a condition may arise from a decrease in the rate of bone synthesis or an increase in the rate of bone destruction or both. The most common form of osteopenia is primary osteoporosis, also referred to as postmenopausal and senile osteoporosis. This form of osteoporosis is a consequence of the universal loss of bone with age and is usually a result of increase in bone resorption with a normal rate of bone formation. About 25 to 30 percent of all white females in the United States develop symptomatic osteoporosis. A direct relationship exists between osteoporosis and the incidence of hip, femoral, neck, and inter-trochanteric fracture in women 45 years and older. Elderly males develop symptomatic osteoporosis between the ages of 50 and 70, but the disease primarily affects females.

The cause of postmenopausal and senile osteoporosis is unknown. Several factors have been identified which may contribute to the condition. They include alteration in hormone levels accompanying aging, and inadequate calcium consumption attributed to decreased intestinal absorption of calcium and other minerals. Treatments have usually included hormone therapy or dietary supplements in an attempt to retard the process. To date, however, an effective treatment for bone loss does not exist.

The invention provides for a method of treating, preventing, or diagnosing the diseases and disorders recited herein using a therapeutically effective amount of TNFr/OPG-like polypeptide. By way of example, the disease or disorder may be any disease or disorder characterized by a net bone loss (such as osteopenia or osteolysis). TNFr/OPG-like polypeptides may be used for example to suppress the rate of bone resorption. Thus, treatment may be done to reduce the rate of bone resorption where the resorption rate is above normal, or to reduce bone resorption to below normal levels in order to compensate for below normal levels of bone formation.

Conditions which are treatable with TNFr/OPG-like polypeptides include the following:

Osteoporosis, such as primary osteoporosis, endocrine osteoporosis (hyperthyroidism, hyperparathryoidism, Cushing's syndrome, and acromegaly), hereditary and congenital forms of osteoporosis (osteogenesis imperfecta, homocystinuria, Menkes' syndrome, and Riley-Day syndrome), and osteoporosis due to immobilization of extremities.

Paget's disease of bone (osteitis deformans) in adults and juveniles;

Osteomyelitis, or an infectious lesion in bone, leading to bone loss;

Hypercalcemia resulting from solid tumors (breast, lung, and kidney) and hematologic malignacies (multiple myeloma, lymphoma, and leukemia), idiopathic hypercalcemia, and hypercalcemia associated with hyperthryoidism and renal function disorders;

Osteopenia following surgery, induced by steroid administration, and associated with disorders of the small and large intestine and with chronic hepatic and renal diseases;

Osteonecrosis, or bone cell death, associated with traumatic injury or nontraumatic necrosis associated with Gaucher's disease, sickle cell anemia, systemic lupus erythematosus, rheumatoid arthritis, periodontal disease, osteolytic metastasis, and other conditions.

Other diseases associated with undesirable levels of one or more of TNF, OPG, IL-1, and/or the present TNFr/OPG-like polypeptide itself are encompassed within the scope of the invention. Undesirable levels include excessive and/or sub-normal levels of TNF, OPG, IL-1, and/or the present TNFr/OPG-like polypeptides described herein.

It is understood that the TNFr/OPG-like polypeptides may be used alone or in conjunction with other factors for the treatment of bone disorders. In one embodiment, TNFr/OPG-like polypeptides are used in conjunction with a therapeutically effective amount of one or more agents which stimulate bone formation or decrease bone destruction. Such agents include, but are not limited to, the bone morphogenic factors (BMP's) designated BMP-1 through BMP-12; transforming growth factor-β (TGF-β) and TGF-βfamily members; interleukin-1 (IL-1) inhibitors; TNF-α inhibitors; parathyroid hormone and analogs thereof, parathyroid related protein and analogs thereof; E series prostaglandins; bisphosphonates (such as alendronate and others); bone-enhancing minerals such as fluoride and calcium; non-steroidal anti-inflammatory drugs (NSAIDs), including COX-2 inhibitors, such as Celebrex™ and Vioxx™; immunosuppressants, such as methotrexate or leflunomide; serine protease inhibitors such as secretory leukocyte protease inhibitor (SLPI); IL-6 inhibitors (e.g., antibodies to IL-6), IL-8 inhibitors (e.g., antibodies to IL-8); IL-18 inhibitors (e.g., IL-18 binding protein or IL-18 antibodies); interleukin-1 converting enzyme (ICE) modulators; fibroblast growth factors FGF-1 to FGF-10 and FGF modulators; PAF antagonists; keratinocyte growth factor (KGF), KGF-related molecules, or KGF modulators; matrix metalloproteinase (MMP) modulators; Nitric oxide synthase (NOS) modulators, including modulators of inducible NOS; modulators of glucocorticoid receptor; modulators of glutamate receptor; modulators of lipopolysaccharide (LPS) levels; and noradrenaline and modulators and mimetics thereof.

A non-exclusive list of acute and chronic diseases treatable in accordance with the invention include, but is not limited to, the following: cachexia/anorexia; cancer (e.g., leukemias); chronic fatigue syndrome; coronary conditions and indications, including congestive heart failure, coronary restenosis, myocardial infarction, and coronary artery bypass graft; depression; diabetes (e.g., juvenile onset Type 1 and diabetes mellitus); endometriosis, endometritis, and related conditions; fibromyalgia or analgesia; graft versus host rejection; hyperalgesia; inflammatory bowel diseases, including Crohn's disease and Clostridium difficile-associated diarrhea; ischemic, including cerebral ischemia (brain injury as a result of trauma, epilepsy, hemorrhage or stroke, each of which may lead to neurodegeneration); lung diseases (e.g., adult respiratory distress syndrome, asthma, and pulmonary fibrosis); multiple sclerosis; neuroinflammatory diseases; ocular diseases and conditions, including corneal transplant, ocular degeneration and uveitis; pain, including cancer-related pain; pancreatitis; periodontal diseases; prostatitis (bacterial or non-bacterial) and related conditions; psoriasis and related conditions; pulmonary fibrosis; reperfusion injury; rheumatic diseases (e.g., rheumatoid arthritis, osteoarthritis, juvenile (rheumatoid) arthritis, seronegative polyarthritis, ankylosing spondylitis, Reiter's syndrome and reactive arthritis, Still's disease, psoriatic arthritis, enteropathic arthritis, polymyositis, dermatomyositis, scleroderma, systemic sclerosis, vasculitis (e.g., Kawasaki's disease), cerebral vasculitis, Lyme disease, staphylococcal-induced (“septic”) arthritis, Sjögren's syndrome, rheumatic fever, polychondritis and polymyalgia rheumatica and giant cell arteritis); septic shock; side effects from radiation therapy; systemic lupus erythematosus; temporal mandibular joint disease; thyroiditis; tissue transplantation or an inflammatory condition resulting from strain, sprain, cartilage damage, trauma, orthopedic surgery, infection (e.g., HIV, Ciostridium difficile and related species) or other disease process.

As contemplated by the present invention, a TNFr/OPG-like polypeptide may be administered as an adjunct to other therapy and also with other pharmaceutical agents suitable for the indication being treated. A TNFr/OPG-like polypeptide and any of one or more additional therapies or pharmaceutical agents may be administered separately, sequentially, or simultaneously.

In a specific embodiment, the present invention is directed to the use of a TNFr/OPG-like polypeptide in combination (pretreatment, post-treatment or concurrent treatment) with any of one or more interleukin-1 inhibitors for the treatment of TNF-responsive disease. Classes of interleukin-1 inhibitors include interleukin-1 receptor antagonists (any compound capable of specifically preventing activation of cellular receptors to IL-1) such as IL-1ra, as described herein; anti-IL-1 receptor monocional antibodies (e.g., EP 623674, the disclosure of which is hereby incorporated by reference); IL-1 binding proteins such as soluble IL-1 receptors (e.g., U.S. Pat. Nos. 5,492,888, 5,488,032, 5,464,937, 5,319,071 and 5,180,812, the disclosures of which are hereby incorporated by reference); anti-IL-1 monoclonal antibodies (e.g., WO 95/01997, WO 94/02627, WO 90/06371, U.S. Pat. No. 4,935,343, EP 364778, EP 267611 and EP 220063, the disclosures of which are hereby incorporated by reference); IL-1 receptor accessory proteins (e.g., WO 96/23067), and other compounds and proteins which block in vivo synthesis or extracellular release of IL-1.

Interleukin-1 receptor antagonist (IL-1ra) is a human protein that acts as a natural inhibitor of interleukin-1. Interleukin-1 receptor antagonists, as well as the methods of making and methods of using thereof, are described in U.S. Pat. No. 5,075,222; WO 91/08285; WO 91/17184; AU 9173636; WO 92/16221; WO 93/21946; WO 94/06457; WO 94/21275; FR 2706772; WO 94/21235; DE 4219626; WO 94/20517; WO 96/22793 and WO 97/28828, the disclosures of which are incorporated herein by reference. The proteins include glycosylated as well as non-glycosylated IL-1 receptor antagonists.

Specifically, three exemplary forms of IL-1ra (IL-1raα, IL-1raβ and IL-1rax), are disclosed and described in U.S. Pat. No. 5,075,222. Methods for producing IL-1 inhibitors, particularly IL-1ras, are also disclosed in the 5,075,222 patent.

An additional class of interleukin-1 inhibitors includes compounds capable of specifically preventing activation of cellular receptors to IL 1. Such compounds include IL-1 binding proteins, such as soluble receptors and monoclonal antibodies. Such compounds also include monoclonal antibodies to the receptors.

A further class of interleukin-1 inhibitors includes compounds and proteins which block in vivo synthesis and/or extracellular release of IL-1. Such compounds include agents which affect transcription of IL-1 genes or processing of IL-1 preproteins.

In a specific embodiment, the present invention is directed to the use of a TNFr/OPG-like polypeptide in combination (pretreatment, post-treatment, or concurrent treatment) with secreted or soluble human fas antigen or recombinant versions thereof (WO 96/20206 and Mountz et al., J. Immunology, 155:4829-4837; and EP 510 691). WO 96/20206 discloses secreted human fas antigen (native and recombinant, including an Ig fusion protein), methods for isolating the genes responsible for coding the soluble recombinant human fas antigen, methods for cloning the gene in suitable vectors and cell types, and methods for expressing the gene to produce the inhibitors. EP 510 691 teaches DNAs coding for human fas antigen, including soluble fas antigen, vectors expressing for said DNAs and transformants transfected with the vector. When administered parenterally, doses of a secreted or soluble fas antigen fusion protein each are generally from about 1 microgram/kg to about 100 micrograms/kg.

Present treatment of diseases associated with TNF, including acute and chronic inflammation such as rheumatic diseases, commonly involves the use of first line drugs for control of pain and inflammation; these drugs are classified as non-steroidal, anti-inflammatory drugs (NSAIDs). Secondary treatments include corticosteroids, slow acting antirheumatic drugs (SAARDs) or disease modifying (DM) drugs. Information regarding the following compounds can be found in The Merck Manual of Diagnosis and Therapy, Sixteenth Edition, Merck, Sharp & Dohme Research Laboratories, Merck & Co., Rahway, N.J. (1992) and in Pharmaprojects, PJB Publications Ltd.

In a specific embodiment, the present invention is directed to the use of a TNFr/OPG-like polypeptide and any of one or more NSAIDs for the treatment of the diseases and disorder recited herein, including acute and chronic inflammation such as rheumatic diseases; and graft versus host disease. NSAIDs owe their anti-inflammatory action, at least in part, to the inhibition of prostaglandin synthesis (Goodman and Gilman in “The Pharmacological Basis of Therapeutics,” MacMillan 7th Edition (1985)). NSAIDs can be characterized into at least nine groups: (1) salicylic acid derivatives; (2) propionic acid derivatives; (3) acetic acid derivatives; (4) fenamic acid derivatives; (5) carboxylic acid derivatives; (6) butyric acid derivatives; (7) oxicams; (8) pyrazoles and (9) pyrazolones.

In another embodiment, the present invention is directed to the use of a TNFr/OPG-like polypeptide in combination (pretreatment, post-treatment, or concurrent treatment) with any of one or more salicylic acid derivatives, prodrug esters or pharmaceutically acceptable salts thereof. Such salicylic acid derivatives, prodrug esters and pharmaceutically acceptable salts thereof comprise: acetaminosalol, aloxiprin, aspirin, benorylate, bromosaligenin, calcium acetylsalicylate, choline magnesium trisalicylate, magnesium salicylate, choline salicylate, diflusinal, etersalate, fendosal, gentisic acid, glycol salicylate, imidazole salicylate, lysine acetylsalicylate, mesalamine, morpholine salicylate, 1-naphthyl salicylate, olsalazine, parsalmide, phenyl acetylsalicylate, phenyl salicylate, salacetamide, salicylamide O-acetic acid, salsalate, sodium_salicylate and sulfasalazine. Structurally related salicylic acid derivatives having similar analgesic and anti-inflammatory properties are also intended to be encompassed by this group.

In an additional specific embodiment, the present invention is directed to the use of a TNFr/OPG-like polypeptide in combination (pretreatment, post-treatment or concurrent treatment) with any of one or more propionic acid derivatives, prodrug esters or pharmaceutically acceptable salts thereof. The propionic acid derivatives, prodrug esters and pharmaceutically acceptable salts thereof comprise: alminoprofen, benoxaprofen, bucloxic acid, carprofen, dexindoprofen, fenoprofen, flunoxaprofen, fluprofen, flurbiprofen, furcloprofen, ibuprofen, ibuprofen aluminum, ibuproxam, indoprofen, isoprofen, ketoprofen, loxoprofen, miroprofen, naproxen, naproxen sodium, oxaprozin, piketoprofen, pimeprofen, pirprofen, pranoprofen, protizinic acid, pyridoxiprofen, suprofen, tiaprofenic acid and tioxaprofen. Structurally related propionic acid derivatives having similar analgesic and anti-inflammatory properties are also intended to be encompassed by this group.

In another specific embodiment, the present invention is directed to the use of a TNFr/OPG-like polypeptide in combination (pretreatment, post-treatment or concurrent treatment) with any of one or more acetic acid derivatives, prodrug esters or pharmaceutically acceptable salts thereof. The acetic acid derivatives, prodrug esters and pharmaceutically acceptable salts thereof comprise: acemetacin, alclofenac, amfenac, bufexamac, cinmetacin, clopirac, delmetacin, diclofenac potassium, diclofenac sodium, etodolac, felbinac, fenclofenac, fenclorac, fenclozic acid, fentiazac, furofenac, glucametacin, ibufenac, indomethacin, isofezolac, isoxepac, lonazolac, metiazinic acid, oxametacin, oxpinac, pimetacin, proglumetacin, sulindac, talmetacin, tiaramide, tiopinac, tolmetin, tolmetin sodium, zidometacin and zomepirac. Structurally related acetic acid derivatives having similar analgesic and anti-inflammatory properties are also intended to be encompassed by this group.

In yet another more specific embodiment, the present invention is directed to the use of a TNFr/OPG-like polypeptide in combination (pretreatment, post-treatment or concurrent treatment) with any of one or more fenamic acid derivatives, prodrug esters or pharmaceutically acceptable salts thereof. The fenamic acid derivatives, prodrug esters and pharmaceutically acceptable salts thereof comprise: enfenamic acid, etofenamate, flufenamic acid, isonixin, meclofenamic acid, meclofenamate sodium, medofenamic acid, mefenamic acid, niflumic acid, talniflumate, terofenamate, tolfenamic acid and ufenamate. Structurally related fenamic acid derivatives having similar analgesic and anti-inflammatory properties are also intended to be encompassed by this group.

In still another more specific embodiment, the present invention is directed to the use of a TNFr/OPG-like polypeptide in combination (pretreatment, post-treatment or concurrent treatment) with any of one or more carboxylic acid derivatives, prodrug esters or pharmaceutically acceptable salts thereof. The carboxylic acid derivatives, prodrug esters and pharmaceutically acceptable salts thereof which can be used comprise: clidanac, diflunisal, flufenisal, inoridine, ketorolac and tinoridine. Structurally related carboxylic acid derivatives having similar analgesic and anti-inflammatory properties are also intended to be encompassed by this group.

In another specific embodiment, the present invention is directed to the use of a TNFr/OPG-like polypeptide in combination (pretreatment, post-treatment or concurrent treatment) with any of one or more butyric acid derivatives, prodrug esters or pharmaceutically acceptable salts thereof. The butyric acid derivatives, prodrug esters and pharmaceutically acceptable salts thereof comprise: bumadizon, butibufen, fenbufen and xenbucin. Structurally related butyric acid derivatives having similar analgesic and anti-inflammatory properties are also intended to be encompassed by this group.

In a further specific embodiment, the present invention is directed to the use of a TNFr/OPG-like polypeptide in combination (pretreatment, post-treatment or concurrent treatment) with any of one or more oxicams, prodrug esters or pharmaceutically acceptable salts thereof. The oxicams, prodrug esters and pharmaceutically acceptable salts thereof comprise: droxicam, enolicam, isoxicam, piroxicam, sudoxicam, tenoxicam and 4-hydroxyl-1,2-benzothiazine 1,1-dioxide 4-(N-phenyl)-carboxamide. Structurally related oxicams having similar analgesic and anti-inflammatory properties are also intended to be encompassed by this group.

In still another specific embodiment, the present invention is directed to the use of a TNFr/OPG-like polypeptide in combination (pretreatment, post-treatment, or concurrent treatment) with any of one or more pyrazoles, prodrug esters or pharmaceutically acceptable salts thereof. The pyrazoles, prodrug esters and pharmaceutically acceptable salts thereof which may be used comprise: difenamizole and epirizole. Structurally related pyrazoles having similar analgesic and anti-inflammatory properties are also intended to be encompassed by this group.

In an additional specific embodiment, the present invention is directed to the use of a TNFr/OPG-like polypeptide in combination (pretreatment, post-treatment or concurrent treatment) with any of one or more pyrazolones, prodrug esters or pharmaceutically acceptable salts thereof. The pyrazolones, prodrug esters and pharmaceutically acceptable salts thereof which may be used comprise: apazone, azapropazone, benzpiperylon, feprazone, mofebutazone, morazone, oxyphenbutazone, phenylbutazone, pipebuzone, propylphenazone, ramifenazone, suxibuzone and thiazolinobutazone. Structurally related pyrazalones having similar analgesic and anti-inflammatory properties are also intended to be encompassed by this group.

In another specific embodiment, the present invention is directed to the use of a TNFr/OPG-like polypeptide in combination (pretreatment, post-treatment or concurrent treatment) with any of one or more of the following NSAIDs: ε-acetamidocaproic acid, S-adenosylmethionine, 3-amino-4-hydroxybutyric acid, amixetrine, anitrazafen, antrafenine, bendazac, bendazac lysinate, benzydamine, beprozin, broperamole, bucolome, bufezolac, ciproquazone, cloximate, dazidamine, deboxamet, detomidine, difenpiramide, difenpyramide, difisalamine, ditazol, emorfazone, fanetizole mesylate, fenflumizole, floctafenine, flumizole, flunixin, fluproquazone, fopirtoline, fosfosal, guaimesal, guaiazolene, isonixirn, lefetamine HCl, leflunomide, lofemizole, lotifazole, lysin clonixinate, meseclazone, nabumetone, nictindole, nimesulide, orgotein, orpanoxin, oxaceprol, oxapadol, paranyline, perisoxal, perisoxal citrate, pifoxime, piproxen, pirazolac, pirfenidone, proquazone, proxazole, thielavin B, tiflamizole, timegadine, tolectin, tolpadol, tryptamid and those designated by company code number such as 480156S, AA861, AD1590, AFP802, AFP860, AI77B, AP504, AU8001, BPPC, BW540C, CHINOIN 127, CN100, EB382, EL508, F1044, FK-506, GV3658, ITF182, KCNTEI6090, KME4, LA2851, MR714, MR897, MY309, ONO3144, PR823, PV102, PV108, R830, RS2131, SCR152, SH440, SIR133, SPAS510, SQ27239, ST281, SY6001, TA60, TAI-901 (4-benzoyl-1-indancarboxylic acid), TVX2706, U60257, UR2301 and WY41770. Structurally related NSAIDs having similar analgesic and anti-inflammatory properties to the NSAIDs are also intended to be encompassed by this group.

In still another specific embodiment, the present invention is directed to the use of a TNFr/OPG-like polypeptide in combination (pretreatment, post-treatment, or concurrent treatment) with any of one or more corticosteroids, prodrug esters or pharmaceutically acceptable salts thereof for the treatment of TNF-responsive diseases, including acute and chronic inflammation such as rheumatic diseases, graft versus host disease and multiple sclerosis. Corticosteroids, prodrug esters and pharmaceutically acceptable salts thereof include hydrocortisone and compounds which are derived from hydrocortisone, such as 21-acetoxypregnenolone, alclomerasone, algestone, amcinonide, beclomethasone, betamethasone, betamethasone valerate, budesonide, chloroprednisone, clobetasol, clobetasol propionate, clobetasone, clobetasone butyrate, clocortolone, cloprednol, corticosterone, cortisone, cortivazol, deflazacon, desonide, desoximerasone, dexamethasone, diflorasone, diflucortolone, difluprednate, enoxolone, fluazacort, flucloronide, flumethasone, flumethasone pivalate, flucinolone acetonide, flunisolide, fluocinonide, fluorocinolone acetonide, fluocortin butyl, fluocortolone, fluocortolone hexanoate, diflucortolone valerate, fluorometholone, fluperolone acetate, fluprednidene acetate, fluprednisolone, flurandenolide, formocortal, halcinonide, halometasone, halopredone acetate, hydrocortamate, hydrocortisone, hydrocortisone acetate, hydrocortisone butyrate, hydrocortisone phosphate, hydrocortisone 21-sodium succinate, hydrocortisone tebutate, mazipredone, medrysone, meprednisone, methylprednisolone, mometasone furoate, paramethasone, prednicarbate, prednisolone, prednisolone 21-diedryaminoacetate, prednisolone sodium phosphate, prednisolone sodium succinate, prednisolone sodium 21-m-sulfobenzoate, prednisolone sodium 21-stearoglycolate, prednisolone tebutate, prednisolone 21-trimethylacetate, prednisone, prednival, prednylidene, prednylidene 21-diethylaminoacetate, tixocortol, triamcinolone, triamcinolone acetonide, triamcinolone benetonide and triamcinolone hexacetonide. Structurally related corticosteroids having similar analgesic and anti-inflammatory properties are also intended to be encompassed by this group.

In another specific embodiment, the present invention is directed to the use of a TNFr/OPG-like polypeptide in combination (pretreatment, post-treatment or concurrent treatment) with any of one or more slow-acting antirheumatic drugs (SAARDs) or disease modifying antirheumatic drugs (DMARDS), prodrug esters or pharmaceutically acceptable salts thereof for the treatment of TNF-responsive diseases, including acute and chronic inflammation such as rheumatic diseases, graft versus host disease and multiple sclerosis. SAARDs or DMARDS, prodrug esters and pharmaceutically acceptable salts thereof comprise: allocupreide sodium, auranofin, aurothioglucose, aurothioglycanide, azathioprine, brequinar sodium, bucillamine, calcium 3-aurothio-2-propanol-1-sulfonate, chlorambucil, chloroquine, clobuzarit, cuproxoline, cyclophosphamide, cyclosporin, dapsone, 15-deoxyspergualin, diacerein, glucosamine, gold salts (e.g., cycloquine gold salt, gold sodium thiomalate, gold sodium thiosulfate), hydroxychloroquine, hydroxychloroquine sulfate, hydroxyurea, kebuzone, levamisole, lobenzarit, melittin, 6-mercaptopurine, methotrexate, mizoribine, mycophenolate mofetil, myoral, nitrogen mustard, D-penicillamine, pyridinol imidazoles such as SKNF86002 and SB203580, rapamycin, thiols, thymopoietin and vincristine. Structurally related SAARDs or DMARDs having similar analgesic and anti-inflammatory properties are also intended to be encompassed by this group.

In a further specific embodiment, the present invention is directed to the use of a TNFr/OPG-like polypeptide in combination (pretreatment, post-treatment, or concurrent treatment) with any of one or more COX2 inhibitors, prodrug esters or pharmaceutically acceptable salts thereof for the treatment of TNF-responsive diseases, including acute and chronic inflammation. Examples of COX2 inhibitors, prodrug esters or pharmaceutically acceptable salts thereof include, for example, celecoxib. Structurally related COX2 inhibitors having similar analgesic and anti-inflammatory properties are also intended to be encompassed by this group.

In yet another specific embodiment, the present invention is directed to the use of a TNFr/OPG-like polypeptide in combination (pretreatment, post-treatment or concurrent treatment) with any of one or more antimicrobials, prodrug esters or pharmaceutically acceptable salts thereof for the treatment of TNF-responsive diseases, including acute and chronic inflammation. Antimicrobials include, for example, the broad classes of penicillins, cephalosporins and other beta-lactams, aminoglycosides, azoles, quinolones, macrolides, rifamycins, tetracyclines, sulfonamides, lincosamides and polymyxins. The penicillins include, but are not limited to penicillin G, penicillin V, methicillin, nafcillin, oxacillin, cloxacillin, dicloxacillin, floxacillin, ampicillin, ampicillin/sulbactam, amoxicillin, amoxicillin/clavulanate, hetacillin, cyclacillin, bacampicillin, carbenicillin, carbenicillin indanyl, ticarcillin, ticarcillin/clavulanate, azlocillin, mezlocillin, peperacillin, and mecillinam. The cephalosporins and other beta-lactams include, but are not limited to cephalothin, cephapirin, cephalexin, cephradine, cefazolin, cefadroxil, cefaclor, cefamandole, cefotetan, cefoxitin, ceruroxime, cefonicid, ceforadine, cefixime, cefotaxime, moxalactam, ceftizoxime, cetriaxone, cephoperazone, ceftazidime, imipenem and aztreonam. The aminoglycosides include, but are not limited to streptomycin, gentamicin, tobramycin, amikacin, netilmicin, kanamycin and neomycin. The azoles include, but are not limited to fluconazole. The quinolones include, but are not limited to nalidixic acid, norfloxacin, enoxacin, ciprofloxacin, ofloxacin, sparfloxacin and temafloxacin. The macrolides include, but are not limited to erythomycin, spiramycin and azithromycin. The rifamycins include, but are not limited to rifampin. The tetracyclines include, but are not limited to spicycline, chlortetracycline, clomocycline, demeclocycline, deoxycycline, guamecycline, lymecycline, meclocycline, methacycline, minocycline, oxytetracycline, penimepicycline, pipacycline, rolitetracycline, sancycline, senociclin and tetracycline. The sulfonamides include, but are not limited to sulfanilamide, sulfamethoxazole, sulfacetamide, sulfadiazine, sulfisoxazole and co-trimoxazole (trimethoprim/sulfamethoxazole). The lincosamides include, but are not limited to clindamycin and lincomycin. The polymyxins (polypeptides) include, but are not limited to polymyxin B and colistin.

In certain preferred embodiments, a polypeptide comprising TNFr/OPG-like polypeptides is used in conjunction with particular therapeutic molecules to treat various inflammatory conditions, autoimmune conditions, and other conditions leading to bone loss. Depending on the condition and the desired level of treatment, two, three, or more agents may be administered, separately, simultaneously, or sequentially. These agents may be provided together by inclusion in the same formulation or inclusion in a treatment kit, or they may be provided separately. When administered by gene therapy, the genes encoding the protein agents may be included in the same vector, optionally under the control of the same promoter region, or in separate vectors. Particularly preferred molecules in the aforementioned classes are as follows.

IL-1 inhibitors: IL-1ra proteins, and soluble IL-1 receptors. The most preferred IL-1 inhibitor is anakinra.

TNF-α inhibitors: soluble tumor necrosis factor receptor type I (sTNF-RI; -RI is also called the p55 receptor); soluble tumor necrosis factor receptor type II (also called the p75 receptor); and monoclonal antibodies that bind the TNF receptor. Most preferred is sTNF-RI as described in WO 98/24463, etanercept (Enbrel®), and Avakine®. Exemplary TNF-α inhibitors are described in EP 422 339, EP 308 378, EP 393 438, EP 398 327, and EP 418 014.

Serine protease inhibitors: Such as SLPI. These inhibitors also may be viewed as exemplary LPS modulators, as SLPI has been shown to inhibit LPS responses. Jin et al. (1997), Cell, 88(3) :417-26.

Particularly preferred methods of treatment concern use of TNF-α inhibitors and IL-1 inhibitors in conjunction with polypeptides comprising TNFr/OPG-like polypeptides. Such polypeptides may be used with either or both TNF-α inhibitors and IL-1 inhibitors for treatment of conditions such as rheumatoid arthritis and multiple sclerosis.

It will be appreciated that other diseases associated with undesirable levels of one or more of TNF, OPG, and/or the present TNFr/OPG-like polypeptides themselves are encompassed within the scope of the invention. Undesirable levels include excessive and/or sub-normal levels of TNF, OPG, and/or the TNFr/OPG-like polypeptides described herein.

TNF-α inhibitors may act by downregulating or inhibiting TNF production, binding free TNF, interfering with TNF binding to its receptor, or interfering with modulation of TNF signaling after binding to its receptor. The term “TNF-α inhibitor” thus includes solubilized TNF receptors, antibodies to TNF, antibodies to TNF receptor, inhibitors of TNF-α converting enzyme (TACE), and other molecules that affect TNF activity.

TNF-α inhibitors of various kinds are disclosed in the art, including the following references:

European patent applications 308 378; 422 339; 393 438; 398 327; 412 486; 418 014, 417 563, 433 900; 464 533;512 528; 526 905; 568 928; 663 210; 542 795; 818 439; 664 128; 542 795; 741 707; 874 819 ; 882 714; 880 970; 648 783; 731 791; 895 988; 550 376; 882 714; 853 083; 550 376; 943 616;

U.S. Pat. Nos. 5,136,021; 5,929,117; 5,948,638; 5,807,862; 5,695,953; 5,834,435; 5,817,822; 5,830,742; 5,834,435; 5,851,556; 5,853,977; 5,359,037; 5,512,544; 5,695,953; 5,811,261; 5,633,145; 5,863,926; 5,866,616; 5,641,673; 5,869,677; 5,869,511; 5,872,146; 5,854,003; 5,856,161; 5,877,222; 5,877,200; 5,877,151; 5,886,010; 5,869,660; 5,859,207; 5,891,883; 5,877,180; 5,955,480; 5,955,476; 5,955,435;

International (WO) patent applications 90/13575, 91/03553, 92/01002, 92/13095, 92/16221, 93/07863, 93/21946, 93/19777, 95/34326, 96/28546, 98/27298, 98/30541, 96/38150, 96/38150, 97/18207, 97/15561, 97/12902, 96/25861, 96/12735, 96/11209, 98/39326, 98/39316, 98/38859, 98/39315, 98/42659, 98/39329, 98/43959, 98/45268, 98/47863, 96/33172, 96/20926, 97/37974, 97/37973, 96/35711, 98/51665, 98/43946, 95/04045, 98/56377, 97/12244, 99/00364, 99/00363, 98/57936, 99/01449, 99/01139, 98/56788, 98/56756, 98/53842, 98/52948, 98/52937, 99/02510, 97/43250, 99/06410, 99/06042, 99/09022, 99/08688, 99/07679, 99/09965, 99/07704, 99/06041, 99/37818, 99/37625, 97/11668;

Japanese (JP) patent applications 10147531, 10231285, 10259140, and 10130149, 10316570, 11001481, and 127,800/1991; German (DE) application 19731521; British (GB) applications 2 218 101, 2 326 881, 2 246 569.

For purposes of this invention, the molecules disclosed in these references including the sTNFRs and variants and derivatives of the sTNFRs disclosed in the references, (see below) are collectively termed “TNF-α inhibitors.”

For example, EP 393 438 and EP 422 339 teach the amino acid and nucleic acid sequences of a soluble TNF receptor type I (also known as sTNFR-I or 30 kDa TNF inhibitor) and a soluble TNF receptor type II (also known as sTNFR-II or 40 kDa TNF inhibitor), collectively termed “sTNFRs”, including modified forms thereof (e.g., fragments, functional derivatives and variants). EP 393 438 and EP 422 339 also disclose methods for isolating the genes responsible for coding the inhibitors, cloning the gene in suitable vectors and cell types, and expressing the gene to produce the inhibitors.

sTNFR-I and sTNFR-II are members of the nerve growth factor/TNF receptor superfamily of receptors which includes the nerve growth factor receptor (NGF), the B cell antigen CD40, 4-1BB, the rat T-cell antigen MRC OX40, the fas antigen, and the CD27 and CD30 antigens (Smith et al. (1990), Science, 248:1019-1023). The most conserved feature among this group of cell surface receptors is the cysteine-rich extracellular ligand binding domain, which can be divided into four repeating motifs of about forty amino acids and which contains 4-6 cysteine residues at positions which are well conserved (Smith et al. (1990), supra).

EP 393 438 teaches a 40 kDa TNF inhibitor Δ51 and a 40 kDa TNF inhibitor Δ53. These are truncated versions of the full-length recombinant 40 kDa TNF inhibitor protein wherein 51 or 53 amino acid residues, respectively, at the carboxyl terminus of the mature protein are removed.

PCT Application No. PCT/US97/12244 teaches truncated forms of sTNFR-I and sTNFR-II which do not contain the fourth domain (amino acid residues Thr¹²⁷-Asn¹⁶¹ of sTNFR-I and amino acid residues Pro¹⁴¹-Thr¹⁷⁹ of sTNFR-II); a portion of the third domain (amino acid residues Asn¹¹¹-Cys¹²⁶ of sTNFR-I and amino acid residues Pro¹²³-Lys¹⁴⁰ of sTNFR-II); and, optionally, which do not contain a portion of the first domain (amino acid residues Asp¹-Cys¹⁹ of sTNFR-I and amino acid residues Leu¹-Cys³² of sTNFR-II).

IL-1 inhibitors include any protein capable of specifically preventing activation of cellular receptors to IL-1, which may result from any number of mechanisms. Such mechanisms include downregulating IL-1 production, binding free IL-1, interfering with IL-1 binding to its receptor, interfering with formation of the IL-1 receptor complex (i.e., association of IL-1 receptor with IL-1 receptor accessory protein), or interfering with modulation of IL-1 signaling after binding to its receptor. Classes of interleukin-1 inhibitors include:

Interleukin-1 receptor antagonists such as IL-1ra, as described herein;

Anti-IL-1 receptor monoclonal antibodies (e.g., EP 623674);

IL-1 binding proteins such as soluble IL-1 receptors (e.g., U.S. Pat. No. 5,492,888, U.S. Pat. No. 5,488,032, and U.S. Pat. No. 5,464,937, U.S. Pat. No. 5,319,071, and U.S. Pat. No. 5,180,812;

Anti-IL-1 monoclonal antibodies (e.g., WO 9501997, WO 9402627, WO 9006371, U.S. Pat. No. 4,935,343, EP 364778, EP 267611 and EP 220063;

IL-1 receptor accessory proteins and antibodies thereto (e.g., WO 96/23067);

Inhibitors of interleukin-1β converting enzyme (ICE) or caspase I, which can be used to inhibit IL-1 beta production and secretion;

Interleukin-1β protease inhibitors;

Other compounds and proteins which block in vivo synthesis or extracellular release of IL-1.

Exemplary IL-1 inhibitors are disclosed in the following references:

U.S. Pat. Nos. 5,747,444; 5,359,032; 5,608,035; 5,843,905; 5,359,032; 5,866,576; 5,869,660; 5,869,315; 5,872,095; 5,955,480;

International (WO) patent applications 98/21957, 96/09323, 91/17184, 96/40907, 98/32733, 98/42325, 98/44940, 98/47892, 98/56377, 99/03837, 99/06426, 99/06042, 91/17249, 98/32733, 98/17661, 97/08174, 95/34326, 99/36426, and 99/36415;

European (EP) patent applications 534978 and 894795; and French patent application FR 2762514.

TNFr/OPG-like Compositions and Administration

Therapeutic compositions are within the scope of the present invention. Such compositions may comprise a therapeutically effective amount of a TNFr/OPG-like polypeptide, including a fragment, variant, derivative, or one or more selective binding agents in admixture with a pharmaceutically acceptable agent such as a pharmaceutically acceptable formulation agent.

TNFr/OPG-like molecule pharmaceutical compositions typically include a therapeutically or prophylactically effective amount of TNFr/OPG-like polypeptide, nucleic acid molecule, or selective binding agent in admixture with one or more pharmaceutically and physiologically acceptable formulation agents selected for suitability with the mode of administration. Suitable formulation materials or pharmaceutically acceptable agents include, but are not limited to, antioxidants, preservatives, coloring, flavoring and diluting agents, emulsifying agents, suspending agents, solvents, fillers, bulking agents, buffers, delivery vehicles, diluents, excipients and/or pharmaceutical adjuvants. For example, a suitable vehicle or carrier may be water for injection, physiological saline solution, or artificial cerebrospinal fluid, possibly supplemented with other materials common in compositions for parenteral administration. Neutral buffered saline or saline mixed with serum albumin are further exemplary vehicles.

The term “pharmaceutically acceptable carrier” or “physiologically acceptable carrier” as used herein refers to one or more formulation materials suitable for accomplishing or enhancing the delivery of the TNFr/OPG-like polypeptide, nucleic acid molecule or selective binding agent as a pharmaceutical composition.

Acceptable formulation materials preferably are nontoxic to recipients and are preferably inert at the dosages and concentrations employed. The materials may include buffers such as phosphate, citrate, or other organic acids; antioxidants such as ascorbic acid; low molecular weight polypeptides; proteins, such as serum albumin, gelatin, or immunoglobulins; hydrophilic polymers such as polyvinylpyrrolidone; amino acids (such as glycine, glutamine, asparagine, arginine or lysine); monosaccharides, disaccharides, and other carbohydrates including glucose, mannose, or dextrins; chelating agents such as ethylenediamine tetraacetic acid (EDTA); sugar alcohols such as mannitol or sorbitol; salt-forming counterions such as sodium; arid/or nonionic surfactants such as tween, pluronics, or polyethylene glycol (PEG).

Typically, a TNFr/OPG-like molecule pharmaceutical composition will be administered in the form of a composition comprising a purified polypeptide, in conjunction with one or more physiologically acceptable agents. It will be appreciated that when used herein, the term “TNFr/OPG-like molecule pharmaceutical composition” also encompasses compositions containing a nucleic acid molecule or selective binding agent of the present invention.

Neutral buffered saline or saline mixed with serum albumin are exemplary appropriate carriers. Other standard pharmaceutically acceptable agents such as diluents and excipients may be included as desired. For example, the TNFr/OPG-like polypeptide product may be formulated as a lyophilizate using appropriate excipients such as sucrose. Other exemplary pharmaceutical compositions comprise Tris buffer of about pH 7.0-8.5, or acetate buffer of about pH 4.0-5.5, which may further include sorbitol or a suitable substitute therefor.

The primary vehicle or carrier in a pharmaceutical composition may be either aqueous or non-aqueous in nature. For example, a suitable vehicle or carrier may be water for injection, physiological saline solution, solution or artificial cerebrospinal fluid, possibly supplemented with other materials common in compositions for parenteral administration. Neutral buffered saline or saline mixed with serum albumin are further exemplary vehicles. Other exemplary pharmaceutical compositions comprise Tris buffer of about pH 7.0-8.5, or acetate buffer of about pH 4.0-5.5, which may further include sorbitol or a suitable substitute therefor. In one embodiment of the present invention, TNFr/OPG-like polypeptide compositions may be prepared for storage by mixing the selected composition having the desired degree of purity with optional formulation agents (Remington's Pharmaceutical Sciences, supra) in the form of a lyophilized cake or an aqueous solution. Further, the TNFr/OPG-like polypeptide product may be formulated as a lyophilizate using appropriate excipients such as sucrose.

In addition, the composition may contain other formulation materials for modifying, maintainingor preserving, for example, the pH, osmolarity, viscosity, clarity, color, sterility, stability, rate of dissolution or release, adsorption or pentration of the composition, or odor of the formulation. Similarly, the composition may contain additional formulation materials for modifying or maintaining the rate of release of TNFr/OPG-like polypeptide, nucleic acid molecule or selective binding agent, or for promoting the absorption or penetration of such TNFr/OPG-like molecules.

The TNFr/OPG-like molecule pharmaceutical compositions can be administered parenterally. Alternatively, the compositions may be administered through the digestive tract, such as orally, or by inhalation. When parenterally administered, the therapeutic compositions for use in this invention may be in the form of a pyrogen-free, parenterally acceptable aqueous solution. The preparation of such pharmaceutically acceptable compositions, with due regard to pH, isotonicity, stability and the like, is within the skill of the art.

A particularly suitable vehicle for parenteral injection is sterile distilled water in which a TNFr/OPG-like polypeptide is formulated as a sterile, isotonic solution, properly preserved. Yet another preparation can involve the formulation of the desired molecule with an agent, such as injectable microspheres, bio-erodible particles or beads, or liposomes, that provides for the controlled or sustained release of the product which may then be delivered as a depot injection. Other suitable means for the introduction of the desired molecule include implantable drug delivery devices.

The pharmaceutical compositions of the present invention may include other components, for example parenterally acceptable preservatives, tonicity agents, cosolvents, wetting agents, complexing agents, buffering agents, antimicrobials, antioxidants (such as ascorbic acid, sodium sulfite or sodium hydrogen-sulfite) and surfactants, as are well known in the art. For example, suitable tonicity enhancing agents include alkali metal halides (preferably sodium or potassium chloride), mannitol, sorbitol, and the like. Suitable preservatives include, but are not limited to, benzalkonium chloride, thimerosal, phenethyl alcohol, methylparaben, propylparaben, chlorhexidine, sorbic acid, and the like. Hydrogen peroxide may also be used as preservative. Suitable cosolvents are for example glycerin, propylene glycol and polyethylene glycol. Suitable complexing agents are for example caffeine, polyvinylpyrrolidone, beta-cyclodextrin or hydroxypropyl-beta-cyclodextrin. Suitable surfactants or wetting agents include sorbitan esters, polysorbates such as polysorbate 80, tromethamine, lecithin, cholesterol, tyloxapal, and the like. The buffers can be conventional buffers such as borate, citrate, phosphate, bicarbonate, or Tris-HCl.

The formulation components are present in concentrations that are acceptable to the site of administration. For example, buffers are used to maintain the composition at physiological pH or at slightly lower pH, typically within a pH range of from about 5 to about 8.

In one embodiment of the present invention, TNFr/OPG-like polypeptide compositions may be prepared for storage by mixing the selected composition having the desired degree of purity with optional physiologically acceptable carriers, excipients, or stabilizers (Remington's Pharmaceutical Sciences, 18^{th} Edition, A. R. Gennaro, ed., Mack Publishing Company [1990]) in the form of a lyophilized cake or an aqueous solution. The optimal pharmaceutical formulation will be determined by one skilled in the art depending upon, for example, the intended route of administration, delivery format, and desired dosage. See for example, Remington's Pharmaceutical Sciences, pp. 1435-1712. Such compositions may influence the physical state, stability, rate of in vivo release, and rate of in vivo clearance of the present TNFr/OPG-like polypeptides.

An effective amount of a TNFr/OPG-like polypeptide composition to be employed therapeutically will depend, for example, upon the therapeutic objectives such as the indication for which the TNFr/OPG-like polypeptide is being used, the route of administration, and the condition of the patient. Accordingly, the clinician may titer the dosage and modify the route of administration to obtain the optimal therapeutic effect. A typical dosage may range from about 0.1 μg/kg to up to about 100 mg/kg or more, depending on the factors mentioned above. In other embodiments, the dosage may range from 1 μg/kg up to about 100 mg/kg; or 5 μg/kg up to about 100 mg/kg; or 0.1 μg/kg up to about 100 mg/kg; or 1 μg/kg up to about 100 mg/kg.

The frequency of dosing will depend upon the pharmokinetic parameters of the TNFr/OPG-like molecule in the formulation used. Typically, a clinician will administer the composition until a dosage is reached that achieves the desired effect. The composition may therefore be administered as a single dose, or as two or more doses (which may or may not contain the same amount of the desired molecule) over time, or as a continuous infusion via implantation device or catheter.

One skilled in the art will appreciate that the appropriate dosage levels for treatment will thus vary depending, in part, upon the molecule delivered, the therapeutic context, type of disorder under treatment, the age, and general health of the recipient.

The TNFr/OPG-like molecule pharmaceutical composition to be used for in vivo administration typically must be sterile. This may be accomplished by filtration through sterile filtration membranes. Where the composition is lyophilized, sterilization using these methods may be conducted either prior to, or following, lyophilization and reconstitution. The composition for parenteral administration may be stored in lyophilized form or in solution. In addition, parenteral compositions generally are placed into a container having a sterile access port, for example, an intravenous solution bag or vial having a stopper pierceable by a hypodermic injection needle.

Once the pharmaceutical composition has been formulated, it may be stored in sterile vials as a solution, suspension, gel, emulsion, solid, or as a dehydrated or lyophilized powder. Such formulations may be stored either in a ready-to-use form or in a form (e.g., lyophilized) requiring reconstitution prior to administration.

In a specific embodiment, the present invention is directed to kits for producing a single-dose administration unit. The kits may each contain both a first container having a dried protein and a second container having an aqueous formulation. Also included within the scope of this invention are kits containing single and multi-chambered pre-filled syringes (e.g., liquid syringes and lyosyringes).

Pharmaceutical compositions such as (1) slow-release formulations, (2) inhalant mists, or (3) orally active formulations are also envisioned. The TNFr/OPG-like molecule pharmaceutical composition generally is formulated for parenteral administration. Such parenterally administered therapeutic compositions are typically in the form of a pyrogen-free, parenterally acceptable aqueous solution comprising the desired TNFr/OPG-like molecule in a pharmaceutically acceptable vehicle. The TNFr/OPG-like molecule pharmaceutical compositions also may include particulate preparations of polymeric compounds such as polylactic acid, polyglycolic acid, etc. or the introduction of the molecule into liposomes. Hyaluronic acid may also be used, and this may have the effect of promoting sustained duration in the circulation.

In one embodiment, a pharmaceutical composition may be formulated for inhalation. For example, TNFr/OPG-like polypeptide may be formulated as a dry powder for inhalation. TNFr/OPG-like polypeptide or nucleic acid molecule inhalation solutions may also be formulated with a propellant for aerosol delivery, with or without a liquefied propellant. In yet another embodiment, solutions may be nebulized. Pulmonary administration is further described in PCT application no. PCT/US94/001875, which describes pulmonary delivery of chemically modified proteins.

It is also contemplated that certain formulations may be administered through the digestive tract, such as orally. In one embodiment of the present invention, TNFr/OPG-like polypeptides which are administered in this fashion can be formulated with or without those carriers customarily used in the compounding of solid dosage forms such as tablets and capsules. For example, a capsule may be designed to release the active portion of the formulation at the point in the gastrointestinal tract when bioavailability is maximized and pre-systemic degradation is minimized. Additional agents can be included to facilitate absorption of the TNFr/OPG-like polypeptide. Diluents, flavorings, low melting point waxes, vegetable oils, lubricants, suspending agents, tablet disintegrating agents, and binders may also be employed.

Another pharmaceutical composition may involve an effective quantity of TNFr/OPG-like molecules in a mixture with non-toxic excipients which are suitable for the manufacture of tablets. By dissolving the tablets in sterile water, or other appropriate vehicle, solutions can be prepared in unit dose form. Suitable excipients include, but are not limited to, inert diluents, such as calcium carbonate, sodium carbonate or bicarbonate, lactose, or calcium phosphate; or binding agents, such as starch, gelatin, or acacia; or lubricating agents such as magnesium stearate, stearic acid, or talc.

Additional TNFr/OPG-like pharmaceutical compositions will be evident to those skilled in the art, including formulations involving TNFr/OPG-like molecules in combination with one or more other therapeutic agents and TNFr/OPG-like polypeptide in sustained release or controlled-delivery formulations. Techniques for formulating a variety of other sustained-or controlled-delivery means, such as liposome carriers, bio-erodible microparticles or porous beads and depot injections, are also known to those skilled in the art. See for example, PCT/US93/00829 which describes controlled release of porous polymeric microparticles for the delivery of pharmaceutical compositions.

Additional examples of sustained-release preparations include semipermeable polymer matrices in the form of shaped articles, e.g. films, or microcapsules. Sustained release matrices may include polyesters, hydrogels, polylactides (U.S. Pat. No. 3,773,919, EP 58,481), copolymers of L-glutamic acid and gamma ethyl-L-glutamate (Sidman et al., Biopolymers, 22:547-556 [1983]), poly (2-hydroxyethyl-methacrylate) (Langer et al., J. Biomed. Mater. Res., 15:167-277 [1981] and Langer, Chem. Tech., 12:98-105 [1982]), ethylene vinyl acetate (Langer et al., supra) or poly-D(−)-3-hydroxybutyric acid (EP 133,988). Sustained-release compositions also may include liposomes, which can be prepared by any of several methods known in the art. See e.g., Eppstein et al., Proc. Natl. Acad. Sci. USA, 82:3688-3692 (1985); EP 36,676; EP 88,046; EP 143,949.

Regardless of the manner of administration, the specific dose may be calculated according to body weight, body surface area or organ size. Further refinement of the appropriate dosage is routinely made by those of ordinary skill in the art and is within the ambit of tasks routinely performed by them. Appropriate dosages may be ascertained through use of appropriate dose-response data.

The route of administration of the pharmaceutical composition is in accord with known methods, e.g. oral, inhalation, injection or infusion by intravenous, intraperitoneal, intracerebral (intra-parenchymal), intracerebroventricular, intramuscular, intraocular, intraarterial, intraportal, or intralesional routes, or by sustained release systems or implantation device. Where desired, the compositions may be administered continuously by infusion, by bolus injection or by implantation device.

Alternatively or additionally, the composition may be administered locally via implantation into the affected area of a membrane, sponge, or other appropriate material on to which the desired molecule has been absorbed or encapsulated. Where an implantation device is used, the device may be implanted into any suitable tissue or organ, and delivery of the desired molecule may be directly through the device via diffusion, time release bolus, or via continuous administration, or via catheter using continuous infusion.

It will further be appreciated that the TNFr/OPG-like polypeptides, including fragments, variants, and derivatives, may be employed alone, together, or in combination with other polypeptides and pharmaceutical compositions. For example, the TNFr/OPG-like polypeptides may be used in combination with cytokines, growth factors, antibiotics, anti-inflammatories, and/or chemotherapeutic agents as is appropriate for the indication being treated.

In some cases, it may be desirable to use TNFr/OPG-like pharmaceutical compositions in an ex vivo manner. In such instances, cells, tissues, or organs that have been removed from the patient are exposed to TNFr/OPG-like pharmaceutical compositions after which the cells, tissues and/or organs are subsequently implanted back into the patient.

In other cases, a TNFr/OPG-like polypeptide can be delivered by implanting certain cells that have been genetically engineered, using methods such as those described herein, to express and secrete the polypeptides. Such cells may be animal or human cells, and may be autologous, heterologous, or xenogeneic. Optionally, the cells may be immortalized. In order to decrease the chance of an immunological response, the cells may be encapsulated to avoid infiltration of surrounding tissues. The encapsulation materials are typically biocompatlble, semi-permeable polymeric enclosures or membranes that allow the release of the protein product(s) but prevent the destruction of the cells by the patient's immune system or by other detrimental factors from the surrounding tissues.

Additional embodiments of the present invention relate to cells and methods (e.g., homologous recombination and/or other recombinant production methods) for both the in vitro production of therapeutic polypeptides by means of homologous recombination and for the production and delivery of therapeutic polypeptides by gene therapy or cell therapy.

It is further envisioned that TNFr/OPG-like polypeptides can be produced in vitro or in vivo by homologous recombination, or with recombinant production methods utilizing control elements introduced into cells already containing DNA encoding TNFr/OPG-like polypeptides. For example, homologous recombination methods may be used to modify a cell that contains a normally transcriptionally silent TNFr/OPG-like gene, or an under expressed gene, and thereby produce a cell which expresses therapeutically efficacious amounts of TNFr/OPG-like polypeptides. Homologous recombination is a technique originally developed for targeting genes to induce or correct mutations in transcriptionally active genes. Kucherlapati, Prog. in Nucl. Acid Res. & Mol. Biol., 36:301, 1989. The basic technique was developed as a method for introducing specific mutations into specific regions of the mammalian genome (Thomas et al., Cell, 44:419-428, 1986; Thomas and Capecchi, Cell, 51:503-512, 1987; Doetschman et al., Proc. Natl. Acad. Sci., 85:8583-8587, 1988) or to correct specific mutations within defective genes (Doetschman et al., Nature, 330:576-578, 1987). Exemplary homologous recombination techniques are described in U.S. Pat. No. 5,272,071 (EP 9193051, EP Publication No. 505500; PCT/US90/07642, International Publication No. WO 91/09955).

Through homologous recombination, the DNA sequence to be inserted into the genome can be directed to a specific region of the gene of interest by attaching it to targeting DNA. The targeting DNA is a nucleotide sequence that is complementary (homologous) to a region of the genomic DNA. Small pieces of targeting DNA that are complementary to a specific region of the genome are put in contact with the parental strand during the DNA replication process. It is a general property of DNA that has been inserted into a cell to hybridize, and therefore, recombine with other pieces of endogenous DNA through shared homologous regions. If this complementary strand is attached to an oligonucleotide that contains a mutation or a different sequence or an additional nucleotide, it too is incorporated into the newly synthesized strand as a result of the recombination. As a result of the proofreading function, it is possible for the new sequence of DNA to serve as the template. Thus, the transferred DNA is incorporated into the genome.

Attached to these pieces of targeting DNA are regions of DNA which may interact with or control the expression of a TNFr/OPG-like polypeptide, e.g., flanking sequences. For example, a promoter/enhancer element, a suppresser, or an exogenous transcription modulatory element is inserted in the genome of the intended host cell in proximity and orientation sufficient to influence the transcription of DNA encoding the desired TNFr/OPG-like polypeptide. The control element controls a portion of the DNA present in the host cell genome. Thus, the expression of TNFr/OPG-like polypeptide may be achieved not by transfection of DNA that encodes the TNFr/OPG-like gene itself, but rather by the use of targeting DNA (containing regions of homology with the endogenous gene of interest) coupled with DNA regulatory segments that provide the endogenous gene sequence with recognizable signals for transcription of a TNFr/OPG-like polypeptide.

In an exemplary method, the expression of a desired targeted gene in a cell (i.e., a desired endogenous cellular gene) is altered via homologous recombination into the cellular genome at a preselected site by the introduction, of DNA which includes at least a regulatory sequence, an exon and a splice donor site. These components are introduced into the chromosomal (genomic) DNA in such a manner that this, in effect, results in the production of a new transcription unit (in which the regulatory sequence, the exon and the splice donor site present in the DNA construct are operatively linked to the endogenous gene). As a result of the introduction of these components into the chromosomal DNA, the expression of the desired endogenous gene is altered.

Altered gene expression, as described herein, encompasses activating (or causing to be expressed) a gene which is normally silent (unexpressed) in the cell as obtained, as well as increasing the expression of a gene which is not expressed at physiologically significant levels in the cell as obtained. The embodiments further encompass changing the pattern of regulation or induction such that it is different from the pattern of regulation or induction that occurs in the cell as obtained, and reducing (including eliminating) the expression of a gene which is expressed in the cell as obtained.

One method by which homologous recombination can be used to increase, or cause, TNFr/OPG-like polypeptide production from a cell's endogenous TNFr/OPG-like gene involves first using homologous recombination to place a recombination sequence from a site-specific recombination system (e.g., Cre/loxP, FLP/FRT) (Sauer, Current Opinion In Biotechnology, 5:521-527, 1994; Sauer, Methods In Enzymology, 225:890-900, 1993) upstream (that is, 5′ to) of the cell's endogenous genomic TNFr/OPG-like polypeptide coding region. A plasmid containing a recombination site homologous to the site that was placed just upstream of the genomic TNFr/OPG-like polypeptide coding region is introduced into the modified cell line along with the appropriate recombinase enzyme. This recombinase causes the plasmid to integrate, via the plasmid's recombination site, into the recombination site located just upstream of the genomic TNFr/OPG-like polypeptide coding region in the cell line (Baubonis and Sauer, Nucleic Acids Res., 21:2025-2029, 1993; O'Gorman et al., Science, 251:1351-1355, 1991). Any flanking sequences known to increase transcription (e.g., enhancer/promoter, intron, translational enhancer), if properly positioned in this plasmid, would integrate in such a manner as to create a new or modified transcriptional unit resulting in de novo or increased TNFr/OPG-like polypeptide production from the cell's endogenous TNFr/OPG-like gene.

A further method to use the cell line in which the site specific recombination sequence had been placed just upstream of the cell's endogenous genomic TNFr/OPG-like polypeptide coding region is to use homologous recombination to introduce a second recombination site elsewhere in the cell line's genome. The appropriate recombinase enzyme is then introduced into the two-recombination-site cell line, causing a recombination event (deletion, inversion, translocation) (Sauer, Current Opinion In Biotechnology, 5:521-527, 1994; Sauer, Methods In Enzymology, 225:890-900, 1993) that would create a new or modified transcriptional unit resulting in de novo or increased TNFr/OPG-like polypeptide production from the cell's endogenous TNFr/OPG-like gene.

An additional approach for increasing, or causing, the expression of TNFr/OPG-like polypeptide from a cell's endogenous TNFr/OPG-like gene involves increasing, or causing, the expression of a gene or genes (e.g., transcription factors) and/or decreasing the expression of a gene or genes (e.g., transcriptional repressors) in a manner which results in de novo or increased TNFr/OPG-like polypeptide production from the cell's endogenous TNFr/OPG-like gene. This method includes the introduction of a non-naturally occurring polypeptide (e.g., a polypeptide comprising a site specific DNA binding domain fused to a transcriptional factor domain) into the cell such that de novo or increased TNFr/OPG-like polypeptide production from the cell's endogenous TNFr/OPG-like gene results.

The present invention further relates to DNA constructs useful in the method of altering expression of a target gene. In certain embodiments, the exemplary DNA constructs comprise: (a) one or more targeting sequences; (b) a regulatory sequence; (c) an exon; and (d) an unpaired splice-donor site. The targeting sequence in the DNA construct directs the integration of elements (a)-(d) into a target gene in a cell such that the elements (b)-(d) are operatively linked to sequences of the endogenous target gene. In another embodiment, the DNA constructs comprise: (a) one or more targeting sequences, (b) a regulatory sequence, (c) an exon, (d) a splice-donor site, (e) an intron, and (f) a splice-acceptor site, wherein the targeting sequence directs the integration of elements (a)-(f) such that the elements of (b)-(f) are operatively linked to the endogenous gene. The targeting sequence is homologous to the preselected site in the cellular chromosomal DNA with which homologous recombination is to occur. In the construct, the exon is generally 3′ of the regulatory sequence and the splice-donor site is 3′ of the exon.

If the sequence of a particular gene is known, such as the nucleic acid sequence encoding a TNFr/OPG-like polypeptide presented herein, a piece of DNA that is complementary to a selected region of the gene can be synthesized or otherwise obtained, such as by appropriate restriction of the native DNA at specific recognition sites bounding the region of interest. This piece serves as a targeting sequence upon insertion into the cell and will hybridize to its homologous region within the genome. If this hybridization occurs during DNA replication, this piece of DNA, and any additional sequence attached thereto, will act as an Okazaki fragment and will be incorporated into the newly synthesized daughter strand of DNA. The present invention, therefore, includes nucleotides encoding a TNFr/OPG-like polypeptide, which nucleotides may be used as targeting sequences.

TNFr/OPG-like polypeptide cell therapy, e.g., the implantation of cells producing TNFr/OPG-like polypeptides, is also contemplated. This embodiment involves implanting cells capable of synthesizing and secreting a biologically active form of TNFr/OPG-like polypeptide. Such TNFr/OPG-like polypeptide-producing cells can be cells that are natural producers of TNFr/OPG-like polypeptides or may be recombinant cells whose ability to produce TNFr/OPG-like polypeptides has been augmented by transformation with a gene encoding the desired TNFr/OPG-like polypeptide or with a gene augmenting the expression of TNFr/OPG-like polypeptide. Such a modification may be accomplished by means of a vector suitable for delivering the gene as well as promoting its expression and secretion. In order to minimize a potential immunological reaction in patients being administered a TNFr/OPG-like polypeptide, as may occur with the administration of a polypeptide of a foreign species, it is preferred that the natural cells producing TNFr/OPG-like polypeptide be of human origin and produce human TNFr/OPG-like polypeptide. Likewise, it is preferred that the recombinant cells producing TNFr/OPG-like polypeptide be transformed with an expression vector containing a gene encoding a human TNFr/OPG-like polypeptide.

Implanted cells may be encapsulated to avoid the infiltration of surrounding tissue. Human or non-human animal cells may be implanted in patients in biocompatible, semipermeable polymeric enclosures or membranes that allow the release of TNFr/OPG-like polypeptide, but that prevent the destruction of the cells by the patient's immune system or by other detrimental factors from the surrounding tissue. Alternatively, the patient's own cells, transformed to produce TNFr/OPG-like polypeptides ex vivo, may be implanted directly into the patient without such encapsulation.

Techniques for the encapsulation of living cells are known in the art, and the preparation of the encapsulated cells and their implantation in patients may be routinely accomplished. For example, Baetge et al. (WO95/05452; PCT/US94/09299) describe membrane capsules containing genetically engineered cells for the effective delivery of biologically active molecules. The capsules are biocompatible and are easily retrievable. The capsules encapsulate cells transfected with recombinant DNA molecules comprising DNA sequences coding for biologically active molecules operatively linked to promoters that are not subject to down regulation in vivo upon implantation into a mammalian host. The devices provide for the delivery of the molecules from living cells to specific sites within a recipient. In addition, see U.S. Pat. Nos. 4,892,538, 5,011,472, and 5,106,627. A system for encapsulating living cells is described in PCT Application WO91/10425 of Aebischer et al. See also, PCT Application WO91/10470 of Aebischer et al., Winn et al., Exper. Neurol., 113:322-329 (1991), Aebischer et al., Exper. Neurol., 111:269-275 (1991); and Tresco et al., ASAIO, 38:17-23 (1992).

In vivo and in vitro gene therapy delivery of TNFr/OPG-like polypeptides is also envisioned. In vivo gene therapy may be accomplished by introducing the gene encoding TNFr/OPG-like polypeptide into cells via local injection of a TNFr/OPG-like nucleic acid molecule or by other appropriate viral or non-viral delivery vectors. Hefti, Neurobiology, 25:1418-1435 (1994). For example, a nucleic acid molecule encoding a TNFr/OPG-like polypeptide may be contained in an adeno-associated virus vector for delivery to the targeted cells (e.g., Johnson, International Publication No. WO95/34670; International Application No. PCT/US95/07178). The recombinant adeno-associated virus (AAV) genome typically contains AAV inverted terminal repeats flanking a DNA sequence encoding a TNFr/OPG-like polypeptide operably linked to functional promoter and polyadenylation sequences.

Alternative suitable viral vectors include, but are not limited to, retrovirus, adenovirus, herpes simplex virus, lentivirus, hepatitis virus, parvovirus, papovavirus, poxvirus, alphavirus, coronavirus, rhabdovirus, paramyxovirus, and papilloma virus vectors. U.S. Pat. No. 5,672,344 describes an in vivo viral-mediated gene transfer system involving a recombinant neurotrophic HSV-1 vector. U.S. Pat. No. 5,399,346 provides examples of a process for providing a patient with a therapeutic protein by the delivery of human cells which have been treated in vitro to insert a DNA segment encoding a therapeutic protein. Additional methods and materials for the practice of gene therapy techniques are described in U.S. Pat. No. 5,631,236 involving adenoviral vectors; U.S. Pat. No. 5,672,510 involving retroviral vectors; and U.S. Pat. No. 5,635,399 involving retroviral vectors expressing cytokines.

Nonviral delivery methods include, but are not limited to, liposome-mediated transfer, naked DNA delivery (direct injection), receptor-mediated transfer (ligand-DNA complex), electroporation, calcium phosphate precipitation, and microparticle bombardment (e.g., gene gun). Gene therapy materials and methods may also include the use of inducible promoters, tissue-specific enhancer-promoters, DNA sequences designed for site-specific integration, DNA sequences capable of providing a selective advantage over the parent cell, labels to identify transformed cells, negative selection systems and expression control systems (safety measures), cell-specific binding agents (for cell targeting), cell-specific internalization factors, and transcription factors to enhance expression by a vector as well as methods of vector manufacture. Such additional methods and materials for the practice of gene therapy techniques are described in U.S. Pat. No. 4,970,154 involving electroporation techniques; WO96/40958 involving nuclear ligands; U.S. Pat. No. 5,679,559 describing a lipoprotein-containing system for gene delivery; U.S. Pat. No. 5,676,954 involving liposome carriers; U.S. Pat. No. 5,593,875 concerning methods for calcium phosphate transfection; and U.S. Pat. No. 4,945,050 wherein biologically active particles are propelled at cells at a speed whereby the particles penetrate the surface of the cells and become incorporated into the interior of the cells.

In yet other embodiments, regulatory elements can be included for the controlled expression of the TNFr/OPG-like gene in the target cell. Such elements are turned on in response to an appropriate effector. In this way, a therapeutic polypeptide can be expressed when desired. One conventional control means involves the use of small molecule dimerizers or rapalogs (as described in WO9641865 (PCT/US96/099486); WO9731898 (PCT/US97/03137) and WO9731899 (PCT/US95/03157)) used to dimerize chimeric proteins which contain a small molecule-binding domain and a domain capable of initiating biological process, such as a DNA-binding protein or transcriptional activation protein. The dimerization of the proteins can be used to initiate transcription of the transgene.

An alternative regulation technology uses a method of storing proteins expressed from the gene of interest inside the cell as an aggregate or cluster. The gene of interest is expressed as a fusion protein that includes a conditional aggregation domain which results in the retention of the aggregated protein in the endoplasmic reticulum. The stored proteins are stable and inactive inside the cell. The proteins can be released, however, by administering a drug (e.g., small molecule ligand) that removes the conditional aggregation domain and thereby specifically breaks apart the aggregates or clusters so that the proteins may be secreted from the cell. See, Science 287:816-817, and 826-830 (2000).

Other suitable control means or gene switches include, but are not limited to, the following systems. Mifepristone (RU486) is used as a progesterone antagonist. The binding of a modified progesterone receptor ligand-binding domain to the progesterone antagonist activates transcription by forming a dimer of two transcription factors which then pass into the nucleus to bind DNA. The ligand binding domain is modified to eliminate the ability of the receptor to bind to the natural ligand. The modified steroid hormone receptor system is further described in U.S. Pat. No. 5,364,791; WO9640911, and WO9710337.

Yet another control system uses ecdysone (a fruit fly steroid hormone) which binds to and activates an ecdysone receptor (cytoplasmic receptor). The receptor then translocates to the nucleus to bind a specific DNA response element (promoter from ecdysone-responsive gene). The ecdysone receptor includes a transactivation domain/DNA-binding domain/ligand-binding domain to initiate transcription. The ecdysone system is further described in U.S. Pat. No. 5,514,578; WO9738117; WO9637609; and WO9303162.

Another control means uses a positive tetracycline-controllable transactivator. This system involves a mutated tet repressor protein DNA-binding domain (mutated tet R-4 amino acid changes which resulted in a reverse tetracycline-regulated transactivator protein, i.e., it binds to a tet operator in the presence of tetracycline) linked to a polypeptide which activates transcription. Such systems are described in U.S. Pat. Nos. 5,464,758; 5,650,298 and 5,654,168.

Additional expression control systems and nucleic acid constructs are described in U.S. Pat. Nos. 5,741,679 and 5,834,186 to Innovir Laboratories Inc.

It is also contemplated that TNFr/OPG-like molecule gene therapy or cell therapy can further include the delivery of a second polypeptide. For example, the host cell may be modified to express and release both TNFr/OPG-like polypeptide and at least one of the following: IL-1ra, sTNFr Type I, sTNFr Type II, and derivatives thereof; Serine Leukocyte Protease Inhibitor (SLPI), Osteoprotogerin (OPG); and anti-TNF antibodies, anti-IL-1 antibodies, and derivatives thereof. Alternatively, the TNFr/OPG-like polypeptide and one or more of the above polypeptides may be expressed in and released from separate cells. Such cells may be separately introduced into the patient, or the cells may be contained in a single implantable device, such as the encapsulating membrane described above, or the cells may be separately modified by means of viral vectors.

One example of a gene therapy technique is to use the TNFr/OPG-like gene (either genomic DNA, cDNA, and/or synthetic DNA encoding a TNFr/OPG-like polypeptide which may be operably linked to a constitutive or inducible promoter to form a “gene therapy DNA construct”. The promoter may be homologous or heterologous to the endogenous TNFr/OPG-like gene, provided that it is active in the cell or tissue type into which the construct will be inserted. Other components of the gene therapy DNA construct may optionally include, DNA molecules designed for site-specific integration (e.g., endogenous sequences useful for homologous recombination), tissue-specific promoter, enhancer(s) or silencer(s), DNA molecules capable of providing a selective advantage over the parent cell, DNA molecules useful as labels to identify transformed cells, negative selection systems, cell specific binding agents (as, for example, for cell targeting), cell-specific internalization factors, and transcription factors to enhance expression by a vector as well as factors to enable vector manufacture.

This gene therapy DNA construct can then be introduced into cells (either ex vivo or in vivo). One means for introducing the gene therapy DNA construct is by means of viral vectors as described herein. Certain vectors, such as retroviral vectors, will deliver the gene therapy DNA construct to the chromosomal DNA of the cells, and the gene therapy DNA construct can integrate into the chromosomal DNA. Other vectors will function as episomes, and the gene therapy DNA construct will remain in the cytoplasm.

Another means to increase endogenous TNFr/OPG-like polypeptide expression in a cell via gene therapy is to insert one or more enhancer elements into the TNFr/OPG-like polypeptide promoter, where the enhancer element(s) can serve to increase transcriptional activity of the TNFr/OPG-like gene. The enhancer element(s) used will be selected based on the tissue in which one desires to activate the gene(s); enhancer elements known to confer promoter activation in that tissue will be selected. For example, if a gene encoding a TNFr/OPG-like polypeptide is to be “turned on” in T-cells, the lck promoter enhancer element may be used. Here, the functional portion of the transcriptional element to be added may be inserted into a fragment of DNA containing the TNFr/OPG-like polypeptide promoter (and optionally, inserted into a vector and/or 5′ and/or 3′ flanking sequence(s), etc.) using standard cloning techniques. This construct, known as a “homologous recombination construct”, can then be introduced into the desired cells either ex vivo or in vivo.

Gene therapy can be used to decrease TNFr/OPG-like polypeptide expression by modifying the nucleotide sequence of the endogenous promoter(s). Such modification is typically accomplished via homologous recombination methods. For example, a DNA molecule containing all or a portion of the promoter of the TNFr/OPG-like gene(s) selected for inactivation can be engineered to remove and/or replace pieces of the promoter that regulate transcription. For example the TATA box and/or the binding site of a transcriptional activator of the promoter may be deleted using standard molecular biology techniques; such deletion can inhibit promoter activity thereby repressing the transcription of the corresponding TNFr/OPG-like gene. The deletion of the TATA box or the transcription activator binding site in the promoter may be accomplished by generating a DNA construct comprising all or the relevant portion of the TNFr/OPG-like polypeptide promoter(s) (from the same or a related species as the TNFr/OPG-like gene(s) to be regulated) in which one or more of the TATA box and/or transcriptional activator binding site nucleotides are mutated via substitution, deletion and/or insertion of one or more nucleotides. As a result, the TATA box and/or activator binding site has decreased activity or is rendered completely inactive. This construct, which also will typically contain at least about 500 bases of DNA that correspond to the native (endogenous) 5′ and 3′ DNA sequences adjacent to the promoter segment that has been modified, may be introduced into the appropriate cells (either ex vivo or in vivo) either directly or via a viral vector as described herein. Typically, the integration of the construct into the genomic DNA of the cells will be via homologous recombination, where the 5′ and 3′ DNA sequences in the promoter construct can serve to help integrate the modified promoter region via hybridization to the endogenous chromosomal DNA.

Other gene therapy methods may also be employed where it is desirable to inhibit the activity of one or more TNFr/OPG-like polypeptides. For example, antisense DNA or RNA molecules, which have a sequence that is complementary to at least a portion of the selected TNFr/OPG-like gene(s) can be introduced into the cell. Typically, each such antisense molecule will be complementary to the start site (5′ end) of each selected TNFr/OPG-like gene. When the antisense molecule then hybridizes to the corresponding TNFr/OPG-like mRNA, translation of this mRNA is prevented or reduced. It will also be appreciated by those skilled in the art that antisense and ribozyme molecules may also be administered directly.

Alternatively, gene therapy may be employed to create a dominant-negative inhibitor of one or more TNFr/OPG-like polypeptides. In this situation, the DNA encoding a mutant full length or truncated polypeptide of each selected TNFr/OPG-like polypeptide can be prepared and introduced into the cells of a patient using either viral or non-viral methods as described herein. Each such mutant is typically designed to compete with endogenous polypeptide in its biological role.

Additional Uses of TNFr/OPG-like Nucleic Acids and Polypeptides

Nucleic acid molecules of the present invention may be used to map the locations of the TNFr/OPG-like gene and related genes on chromosomes. Mapping may be done by techniques known in the art, such as PCR amplification and in situ hybridization.

The nucleic acid molecules are also used as anti-sense inhibitors of TNFr/OPG-like polypeptide expression. Such inhibition may be effected by nucleic acid molecules which are complementary to and hybridize to expression control sequences (triple helix formation) or to TNFr/OPG-like mRNA. Anti-sense probes may be designed by available techniques using the sequence of TNFr/OPG-like nucleic acid molecules disclosed herein. Anti-sense inhibitors provide information relating to the decrease or absence of a TNFr/OPG-like polypeptide in a cell or organism.

Hybridization probes may be prepared using the TNFr/OPG-like nucleic acid sequences provided herein to screen cDNA, genomic or synthetic DNA libraries for related sequences. Regions of the DNA and/or amino acid sequence of TNFr/OPG-like polypeptide that exhibit significant identity to known sequences are readily determined using sequence alignment algorithms as described herein and those regions may be used to design probes for screening.

TNFr/OPG-like nucleic acid molecules, as well as fragments, variants, and/or derivatives that do not themselves encode biologically active polypeptides, may be useful as hybridization probes in diagnostic assays to test, either qualitatively or quantitatively, for the presence of TNFr/OPG-like DNA or corresponding RNA in mammalian tissue or bodily fluid samples.

The TNFr/OPG-like polypeptides may be used (simultaneously or sequentially) in combination with one or more cytokines, growth factors, antibiotics, anti-inflammatories, and/or chemotherapeutic agents as is appropriate for the indication being treated.

TNFr/OPG-like polypeptide fragments, variants, and/or derivatives, whether biologically active or not, are also useful for preparing antibodies that bind to a TNFr/OPG-like polypeptide. The antibodies may be used for in vivo and in vitro diagnostic purposes, including, but not limited to, use in labeled form to detect the presence of TNFr/OPG-like polypeptide in a body fluid or cell sample. The antibodies may also be used to prevent or treat the diseases and disorders recited herein. The antibodies may bind to a TNFr/OPG-like polypeptide so as to diminish or block at least one activity characteristic of a TNFr/OPG-like polypeptide, or may bind to a polypeptide to increase an activity.

The following example will serve to further typify the nature of the invention but should not be construed as a limitation on the scope thereof which is defined solely by the appended claims.

### EXAMPLE 1

### Cloning of TNFr/OPG-like cDNA

Homology-based BLAST searches of a human genomic database identified a 543 nucleotide genomic DNA fragment (SEQ ID NO: 5) which upon translation displayed homology to the known human OPG polypeptide sequence. Based upon this sequence information, nucleotide primers 2374-51 (5′-CCC CAG GCA CCT TCT CAG CTG C-3′ SEQ ID NO: 9) and 2374-52 (5′-GTG TAT CTC GAG TTG CCA TGC CC-3′; SEQ ID NO: 10) were synthesized and used to screen a variety of human cDNA libraries. Using PCR beads (Pharmacia, Piscataway, N.J.), a final reaction volume of 25 μl, and 10 pmol of each oligonucleotide, the expected size band of 111 nucleotides (nt) was identified in a number of libraries including fetal scalp (both random and oligo dT primed) and fetal spleen (both random and oligo dT primed. The cycling conditions were 94° C. for 1 min, (94° C. for 30 sec, 68° C. for 45 sec) repeat 35 times, then 72° C. for 10 minutes.

Based upon this, to isolate the 5′ region of the cDNA, PCR was performed on the fetal spleen and fetal scalp cDNA libraries using pSPORT (LTI) vector primers 870-02 (5-AGC GGA TAA CAA TTT CAC ACA GG-3′; SEQ ID NO: 11) and 1916-83 (5-GGC TCG TAT GTT GTG TGG AAT TGT GAG CG-3′; SEQ ID NO: 12), and a gene specific primer 2374-53 (5′-CCC AGG CCA GCA GTC TCC ACA G -3′; SEQ ID NO: 13) using Clontech Advantage PCR Mix (Clontech, Palo alto, Calif.). The cycling conditions were as follows: 94° C. for 1 min; 94° C. for 5 sec; 72° C. for 3 min; (Repeated 5 times); followed by: 94° C. for 5 sec; 70° C. for 3 min; (Repeated 5 times; followed by: 94° C. for 5 sec; 68° C. for 3 min; Repeated 25 times; 72° C. for 10 min. PCR products were obtained from these cDNA libraries.

The PCR products obtained in these reactions were diluted 1:100 and PCR amplified with nested vector primers 1019-06 (5′-GCT CTA ATA CGA CTC ACT ATA GGG-3′; SEQ ID NO: 14) and 1916-82 (5′-CAT GAT TAC GCC AAG CTC TAA TAC GAC TC-3′; SEQ ID NO: 15), and a nested gene specific primer 2374-52 (5′-GTG TAT CTC GAG TTG CCA TGC CC-3′; SEQ ID NO: 10). The specific PCR products were subcloned into pGEM-T (Promega, Madison, Wis.) using the TA cloning protocol according to the manufacturer's instructions. The 3′ region was isolated by PCR amplification of fetal scalp cDNA library using a vector primers 1340-35 (5′-CCC AGT CAC GAC GTT GTA AAA CG-3′: SEQ ID NO: 16) and a gene specific primer 2374-51 (5-CCC CAG GCA CCT TCT CAG CTG C-3′; SEQ ID NO: 9) using Clontech Advantage PCR Mix (Clontech, Palo alto, Calif.). The cycling conditions were 94° C. for 2 min, (94° C. for 15 sec, 66° C. for 15 sec, and 72° C. for 3 min) repeated 35 times, 72° C. for 2 min and then kept at 4° C. until being analyzed. The PCR products obtained in the reaction were diluted 1:100 and PCR amplified with a nested vector primer 1019-05 (5′- TGA ATT TAG GTG ACA CTA TAG AAG AG-3′: SEQ ID NO: 17) and a nested gene specific primer 2374-78 (5′-GCC CGT TGC AGC CTT TGG AG-3′: SEQ ID NO: 18) using Clontech Advantage PCR Mix (Clontech, Palo alto, Calif.). The cycling conditions were the same as mentioned above. The final PCR products were subcloned into pGEM-T (Promega, Madison, Wis.) using the TA cloning protocol. The sequence of the 5′ RACE clones and 3′ region was determined by DNA sequencing using standard methods known to those skilled in the art. The sequence was assembled and found to encode a protein of 430 amino acids in length.

The cDNA libraries utilized to isolate this TNFr/OPG-like gene were made as follows. Total RNA was extracted from human tissue using standard RNA extraction procedures and poly-A⁺ RNA was selected from this total RNA using standard procedures known to those skilled in the art. Random primed or oligo(dT) primed cDNA was synthesized from this poly-A⁺ RNA using the procedure in the manual of the Superscript Plasmid System for cDNA Synthesis and Plasmid Cloning kit (Gibco-BRL, Inc., Rockville, Md.) or using other suitable procedures known to those skilled in the art. The resulting cDNA was digested with appropriate restriction enzymes (SalI and NotI) to create sticky ends to assist in ligation to a cloning vector. This digested cDNA was then ligated into the pSPORT-1 cloning vector, or another suitable cloning vector known to those skilled in the art, that had been pre-digested with appropriate restriction enzymes. The ligation products were transformed into E. coli using standard techniques known in the art, and transformants were selected on bacterial media plates containing ampicillin. The cDNA library consisted of all, or a subset, of these transformants.

### EXAMPLE 2

### Evaluation of TNFr/OPG Tissue Expression

Methods for mRNA expression analysis by RT-PCR were as follows.

Reverse transcription (RT) reactions. 2 ug of total RNA from each human fetal tissue (total RNAs were purified by Total RNA Isolation Kit from Amersham Pharmacia Biotech Inc., Cat.# 15593-031). The reaction Mixture contained 2 ug total RNA, and 1 ul (1 ug) Random Primer. The volume was adjusted to 12 ul with water, heated to 70° C. for 10 min, and quick-chilled on ice. 4 ul 5×First Stand Buffer (BRL), 2 ul 0.1 M DTT (BRL), and 1 ul 10 mM DNTP Mix(BRL) were then added, and the solution was mixed well and warmed to 37° C. for 2 min. 1 ul Superscript II RT (BRL) was added, and the solution was incubated at 37° C. for 1 hr.

The reaction tube was then placed in ice to terminate the reaction. cDNAs produced in this way were used as the template in the PCR analysis.

Estimate of Relative Expression Levels

In order to normalize for differences in RNA concentration and cDNA conversion efficiency, control PCRs were performed on each cDNA using primers to Glyceraldehyde-3-phosphate dehydrogenase (G3PDH), a gene expected to be expressed at about the same level in all tissues. The products of this reaction were analyzed on 4% agarose gels and the relative intensity of the control bands were estimated. cDNA samples were then diluted according to the intensity of the control bands so that all samples were adjusted to a concentration that would produce G3PDH control bands of equal intensity. Expression analysis for the OPG-like transcript was done using these concentration-normalized samples.

G3PDH Control PCRs

Template: 1 μl of cDNA (prior to concentration adjustment)

| | |
|---|---|
| Primers: | |
| 5′primer:5′-TCCACCACCCTGTTGCTGTAG-3′ | SEQ ID NO:19 |
| 3′primer:5′-GACCACAGTCCATCCCATCACT-3′ | SEQ ID NO:20 |

Buffer/enzyme: Ready-To-Go PCR Beads (Amersham Pharmacia Biotech Inc., Cat. # 27-95530)

Cycling protocol:

95° C. 60 sec;

92° C. 30 sec, 55° C. 45 sec, 72° C. 60 sec, 25 cycles;

72° C. 5 min.

Relative Expression Levels of OPG-like Transcript

Template: 1 μl of cDNA (concentration adjusted as described above).

| | |
|---|---|
| Primers: (2374-51) | |
| 5′-CCCCAGGCACCTTCTCAGCTGC-3′ | SEQ ID NO:9 |
| (2374-53) | |
| 5′-CCCAGGCCAGCAGTCTCCACAG-3′ | SEQ ID NO:13 |

Buffer/enzyme: Ready-To-GO PCR Beads from Amersham Pharmacia Biotech Inc. (Cat. # 27-95530).

Cycling protocol:

95° C. 30 sec;

94° C. 5 sec, 72° C. 4 min, 5 cycles;

94° C. 5 sec, 70° C. 4 min, 5 cycles;

94° C. 5 sec, 68° C. 2 min, 25 cycles;

72° C. 3 min.

Products were run on 4% agarose/TBE gel electrophoresis. Using the faintest band as a baseline (1X), the relative intensity of the band corresponding to the amplified OPG-like transcript was then estimated. The estimated relative intensity of each band is set forth below, with highest intensities being found in fetal tissue, fetal uterus and fetal skin.

| # | Tissue | Expression Level |
|---|---|---|
| 1 | Fetal Stomach | XXX |
| 2 | Fetal Pancreas | XXX |
| 3 | Fetal Bladder | XXX |
| 4 | Fetal Brain | XXX |
| 5 | Lymphoma Cell | XX |
| | Lines | |
| 6 | Fetal Testis | XXXXX |
| 7 | Fetal Thymus | XXXX |
| 8 | Fetal Placenta | XX |
| 9 | Fetal Spinal Cord | XXX |
| 10 | Fetal Heart | X |
| 11 | Fetal Uterus | XXXXX |
| 12 | Fetal Kidney | XXXX |
| 13 | Fetal Skin | XXXXX |
| 14 | Fetal Liver | XXX |
| 15 | Fetal Lung | XXX |
| 16 | Fetal Mesentery | XX |
| 17 | Fetal Bone | XX |

### EXAMPLE 3

### Production of TNFr/OPG-like Polypeptides

A. Bacterial Expression

PCR is used to amplify template DNA sequences encoding a polypeptide using primers corresponding to the 5′ and 3° ends of the sequence. The amplified DNA products may be modified to contain restriction enzyme sites to allow for insertion into expression vectors. PCR products are gel purified and inserted into expression vectors using standard recombinant DNA methodology. An exemplary vector, such as pAMG21 (ATCC No. 98113) containing the lux promoter and a gene encoding kanamycin resistance is digested with BamHI and NdeI for directional cloning of inserted DNA. The ligated mixture is transformed into an E. coli host strain by electroporation and transformants are selected for kanamycin resistance. Plasmid DNA from selected colonies is isolated and subjected to DNA sequencing to confirm the presence of the insert.

Transformed host cells are incubated in 2×YT medium containing 30 g/ml kanamycin at 30° C. prior to induction. Gene expression is induced by the addition of N-(3-oxohexanoyl)-dl-homoserine lactone to a final concentration of 30 ng/ml followed by incubation at either 30° C. or 37° C. for six hours. The expression of TNFr/OPG-like polypeptide is evaluated by centrifugation of the culture, resuspension and lysis of the bacterial pellets, and analysis of host cell proteins by SDS-polyacrylamide gel electrophoresis.

Inclusion bodies containing TNFr/OPG-like polypeptide are purified as follows. Bacterial cells are pelleted by centrifugation and resuspended in water. The cell suspension is lysed by sonication and pelleted by centrifugation at 195,000×g for 5 to 10 minutes. The supernatant is discarded, and the pellet is washed and transferred to a homogenizer. The pellet is homogenized in 5 ml of a Percoll solution (75% liquid Percoll. 0.15M NaCl) until uniformly suspended and then diluted and centrifuged at 21,600×g for 30 minutes. Gradient fractions containing the inclusion bodies are recovered and pooled. The isolated inclusion bodies are analyzed by SDS-PAGE.

A single band on an SDS polyacrylamide gel corresponding to E. coli produced TNFr/OPG-like polypeptide is excised from the gel, and the N-terminal amino acid sequence is determined essentially as described by Matsudaira et al., J. Biol. Chem., 262:10-35 (1987).

B. Mammalian Cell Production

PCR is used to amplify template DNA sequences encoding a TNFr/OPG-like polypeptide using primers corresponding to the 5′ and 3′ ends of the sequence. The primer sequences corresponding to the 5′ and 3′ ends are described above. The amplified DNA products may be modified to contain restriction enzyme sites to allow for insertion into expression vectors. PCR products are gel purified and inserted into expression vectors using standard recombinant DNA methodology. An exemplary expression vector, pCEP4 (Invitrogen, Carlsbad, Calif.), which contains an Epstein-Barr virus origin of replication, may be used for the expression of TNFr/OPG-like in 293-EBNA-1 (Epstein-Barr virus nuclear antigen) cells. Amplified and gel purified PCR products are ligated into pCEP4 vector and lipofected into 293-EBNA cells. The transfected cells are selected in 100 g/ml hygromycin and the resulting drug-resistant cultures are grown to confluence. The cells are then cultured in serum-free media for 72 hours. The conditioned media is removed and, TNFr/OPG-like polypeptide expression is analyzed by SDS-PAGE.

TNFr/OPG-like polypeptide expression may be detected by silver staining. Alternatively, TNFr/OPG-like polypeptide is produced as a fusion protein with an epitope tag, such as an IgG constant domain or a FLAG epitope, which may be detected by Western blot analysis using antibodies to the tag peptide.

TNFr/OPG-like polypeptides may be excised from an SDS-polyacrylamide gel, or TNFr/OPG-like fusion proteins are purified by affinity chromatography to the epitope tag, and subjected to N-terminal amino acid sequence analysis as described herein.

### EXAMPLE 4

### Production of Anti-TNFr/OPG-like Polypeptide Antibodies

Antibodies to TNFr/OPG-like polypeptides may be obtained by immunization with purified protein or with TNFr/OPG-like peptides produced by biological or chemical synthesis. Suitable procedures for generating antibodies include those described in Hudson and Hay, Practical Immunology, 2nd Edition, Blackwell Scientific Publications (1980).

In one procedure for the production of antibodies, animals (typically mice or rabbits) are injected with a TNFr/OPG-like antigen (such as a TNFr/OPG-like polypeptide), and those with sufficient serum titer levels as determined by ELISA are selected for hybridoma production. Spleens of immunized animals are collected and prepared as single cell suspensions from which splenocytes are recovered. The splenocytes are fused to mouse myeloma cells (such as Sp2/0-Ag14 cells; ATCC no. CRL-1581), allowed to incubate in DMEM with 200 U/ml penicillin, 200 g/ml streptomycin sulfate, and 4 mM glutamine, then incubated in HAT selection medium (Hypoxanthine; Aminopterin; Thymidine). After selection, the tissue culture supernatants are taken from each well containing a hybridoma and tested for anti-TNFr/OPG-like antibody production by ELISA.

Alternative procedures for obtaining anti-TNFr/OPG-like antibodies may also be employed, such as the immunization of transgenic mice harboring human Ig loci for the production of human antibodies, and the screening of synthetic antibody libraries, such as those generated by mutagenesis of an antibody variable domain.

### EXAMPLE 5

### Production of TNFr/OPG-like Protein in Mammalian Cells

To generate soluble TNFr/OPG-like protein in mammalian cells, the cDNA encoding the extracellular domain of human TNFr/OPG-like polypeptide (amino acid 1-162) was PCR amplified with the following set of oligo primer pair:

5′-CCA TCG ATG GCT GAG CAG CAG GTG TGG ACA-3′ (SEQ ID NO: 21)

5′-TGG CGA TGA CGG TGA CCT GGG CGG-3′ (SEQ ID NO: 22).

The PCR reaction was carried out in a 50 μl volume which consisted of 1 unit of vent DNA polymerase (New England Biolabs) in 20 mM Tris-HCl pH 8.8, 10 mM KCl, 10 μM (NH₄)₂SO₄, 0.1% Triton-X100, 10 μM of each dNTP, 1 μM of each primer and 10 ng of TNFr/OPG-like cDNA template. PCR Reactions were performed at 98° C. for 30 seconds, 55° C. for 30 seconds, and 72° C. for 1 minute, for a total of 5 cycles and 98° for 30 seconds, 65° for 30 seconds, 72° for 1 minute for a total of 25 cycles The resulting PCR fragment was isolated by electrophoresis through 1% agarose gel and purification by the Geneclean procedure (Bio 101, Inc.). The PCR fragment creates a Cla I restriction site at its 5′ end and a BstEII restriction site at its 3′ end. The ClaI+BstEII digested PCR fragment was then subcloned in-frame into a modified pCMVi-Fc vector in front of the human IgG-γ1 heavy chain sequence as described previously by Vasser et al. (Science 286, pp. 735-741, 1999). A linker was introduced which encodes two irrelevant amino acids (Val-Thr) spanning the junction between the TNFr/OPG-like extracellular domain and the IgG Fc region.

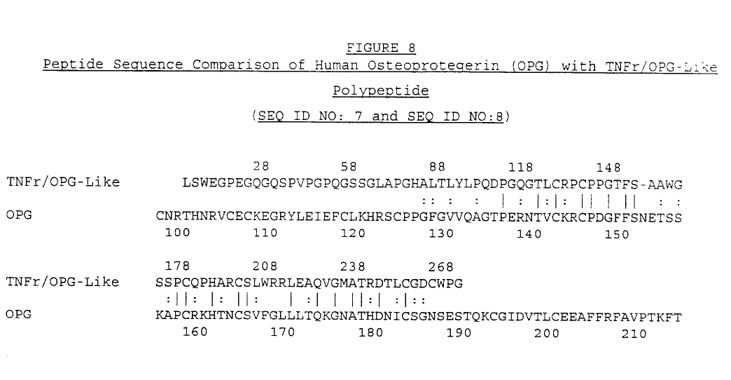

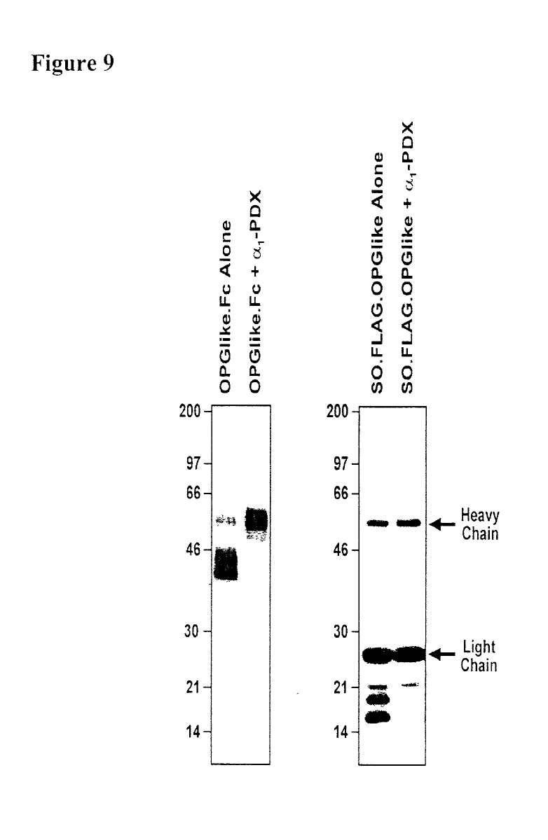

The construct was transfected into 293-T cells by the calcium phosphate method as described by Ausubel et al. (Curr. Prot. Mol. Biol. 1, 9.1.1-9.1.3, 1994). Twenty-four hours post-transfection, the cells were washed in PBS once and then cultured in serum-free media for 72 hr. The conditioned media was collected. The TNFr/OPG-like-Fc fusion protein that was secreted into the media was detected by Western blot analysis with anti-human IgG Fc antibody (Jackson Immuno Reasearch cat no. 309-035-008) (FIG. 9) and three distinct bands were observed having molecular weights, 56.6 kD, 44.3 kD, and 40.6 kD respectively.

The Fc fusion protein was purified by protein-A column chromatography (Pierce) according to the manufacturer's recommended procedures. Fifty pmoles of the purified protein was then subjected to N-terminal sequence analysis by automated Edman degradation as essentially described by Matsudaira et al. (J. Biol. Chem. 262, 10-35, 1987).

Following 10 cycles of amino acid sequencing, the 56.6 kD band gave the sequence NH₂-ST(T)LWQCPPGEE-CO₂H (1.4 pmol)(SEQ ID NO: 23). At the third cycle, Thr was not detected as expected from the primary structure of the protein, indicating the possibility of O-linked sugars. The results show that the protein was cleaved at Thr25. The 44.3 kD band (14.6 pmol) and the 40.6 kD band (24.7 pmol) both gave the sequence NH₂-GVEVAAGASSGGET-CO₂H (SEQ ID NO: 24); indicating the protein was cleaved at Arg130. The difference in size between these two bands is presumably due to differential N-linked glycosylation at Arg149. The 40.6 kD band and the 44.3 kD band represent approximately 97% of the recovered material. Closer examination of the cleavage site at Arg 130 reveals a consensus furin cleavage site beginning with Arg126 (RRARR-GVEV . . . ) (SEQ ID NO: 25).

To explore the role of furin in the cleavage of TNFr/OPG-like receptor extracellular domain, we transiently transfected 293-T cells with TNFr/OPG-like-Fc with or without co-transfection of the potent furin inhibitor α1-antitrypsin containing the Portland mutation (α1-PDX) ( J. Biol. Chem.: 24887-91.1993). In short, 7×10⁶ 293-T cells were transiently transfected with 20 μgs of TNFr/OPG-like-Fc alone or with 15 μgs of TNFr/OPG-like-Fc and 5 μgs of α1-PDX, using the CaOPO₄ method of transfection described above. The conditioned medium was collected and subjected to Western blot analysis as described above. Co-transfection with α1-PDX completely abrogated furin cleavage resulting in 100% of the recovered material beginning with Ser26 as shown in FIG. 9 (left panel).

To further confirm the role of furin cleavage in liberation of a soluble extracellular domain of TNFr/OPG-like receptor, we engineered a version of the TNFr/OPG-like receptor containing the signal peptide from OPG and an in frame, NH2 terminal FLAG epitope tag (SO.FLAG-TNFr/OPG-like receptor). The SO.FLAG-TNFr/OPG-like receptor construct encodes a protein containing OPG signal peptide (amino acid 1-21)-linker(KLH)-FLAG epitope (MDYKDDDDK; SEQ ID NO: 26)-linker (KL)-TNFr/OPG-like receptor (amino acid 26-430). Again, 7×10⁶ 293-T cells were transfected with SO.FLAG-TNFr/OPG-like receptor alone or co-transfected with α1-PDX. Twnet-four hours after the transfection, cells were incubated in serum free media for 72 hours. The conditioned media was collected and analyzed by immunoprecipitation/Western blotting using the anti-FLAG monoclonal antibody M2 (Sigma, St. Louis Mo.). Two distinct bands of 17 KDa and 18 Kda were detected in the conditioned medium, corresponding to the cleaved soluble extracellular domain of OPG-like receptor as shown in FIG. 9 (left panel). Similarly co-transfection with α1-PDX dramatically reduces the amount of shed FLAG-TNFr/OPG-like extracellular domain recovered from the conditioned media.

### EXAMPLE 6

### Detection of TNFr/OPG-like-Fc Binding to WEHI-3 Cells

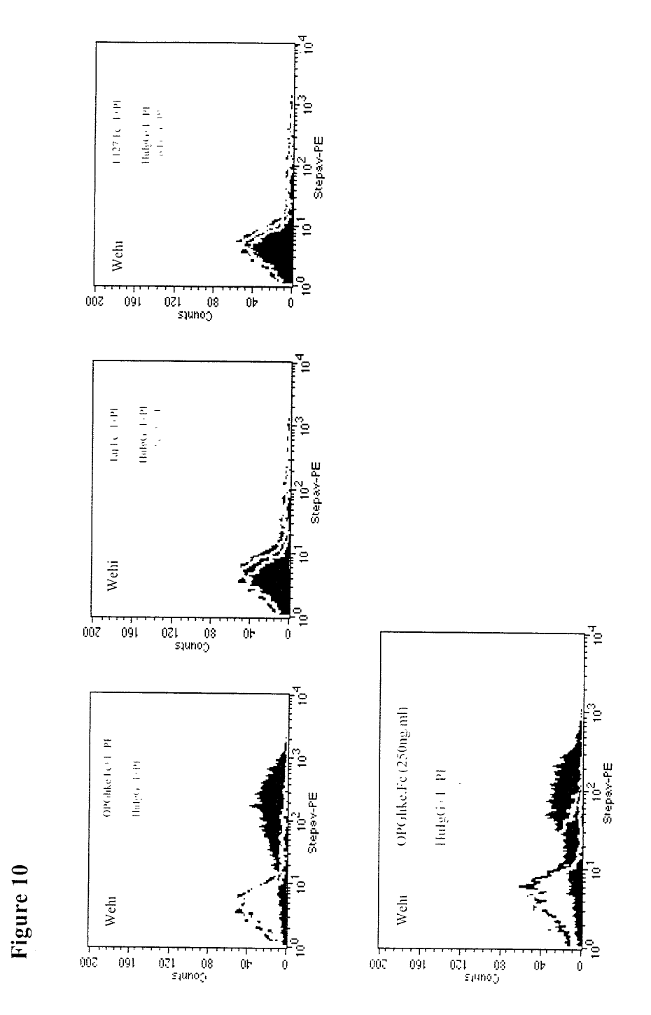

The binding activity of TNFr/OPG-like-Fc with various cell lines was tested by FACS analysis as previously described (Goodwin et al. Cell, 73, 447-456, 1993). Briefly, WEHI-3 cells were incubated for 30 minutes at 40° C. in PBS supplemented with 2% rabbit serum and 5% goat serum for blocking purposes. Subsequently, the cells were incubated with 1 μg/ml TNFr/OPG-like-Fc fusion protein or human IgG. The cells were then stained for 30 minutes at 40° C. with biotinylated antibody specific for the Fc domain of human IgG (Jackson Immunoresearch,West Grove, Pa.) at a dilution of 1:200, followed by a 30 minute incubation with streptavidin-phycoerythrin (Jackson Immunoresearch,West Grove, Pa.) at a dilution of 1:50. The cells were then subjected to FACS analysis using a Becton Dickenson FACS Scan. TAJ.FC and E127.FC were non-specific fusion proteins used for controls and did not result in any specific binding. This analysis revealed that WEHI-3 cells, a myelo-monocytic cell line, specifically bound the TNFr/OPG-like-Fc fusion protein indicating the presence of a membrane bound form of a putative ligand for the OPGlike receptor.(See FIG. 10) The binding of TNFr/OPG-like-Fc fusion protein was partially blocked by pre-incubation with conditioned media containing the N-terminal FLAG tagged TNFr/OPG-like extracellular domain.

### EXAMPLE 7

### Northern Blot Analysis of TNFr/OPG-like Receptor mRNA Tissue Expression

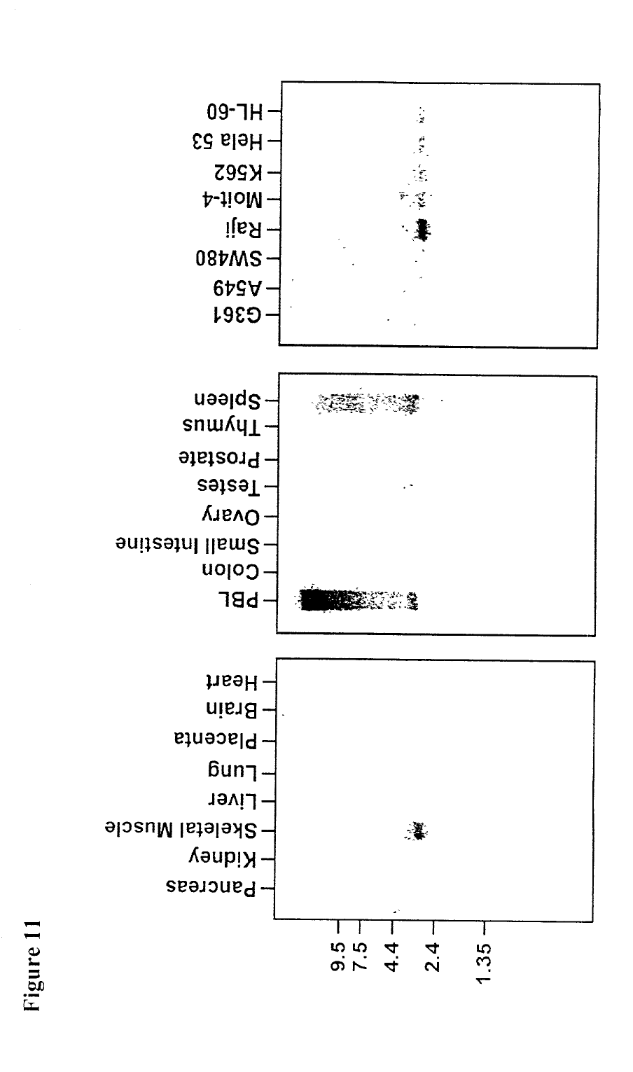

Northern blot analysis was performed to identify those tissues in which the TNFr/OPG-like receptor transcript is expressed. A probe for use in Northern blot analysis was generated by digesting the human TNFr/OPG-like receptor cDNA with EcoRV and XhoI for about three hours at 37° C. and running the restriction digest on an 0.8% agarose gel to separate the fragments. An approximately 434 base pair ECORV-XhoI fragment, extending from nucleotide −180 to nucleotide +254 of the cDNA,was isolated and gel purified using the QiaQuick® gel purification system (Qiagen, Chatsworth, Calif.) The isolated, gel purified fragment was quantitated by estimation on a one percent agarose gel. About 25 ng of this fragment was denatured by boiling for 5 minutes, and then quenching on ice for 2 minutes. The fragment was then radioactively labeled with α-³²P-dCTP using the High Prime DNA labeling kit (Boehringer Manheim, Indianapolis, Ind.) according to the manufacturer's protocol. Human multiple tissue northern blots were purchased (Clonetech, Palo Alto, Calif.) and first prehybridized in Clontech Express™ hybridization buffer for about one hour at about 65° C. Following prehbridization, the labeled probe was denatured by boiling for about five minutes then quenched on ice for 2 minutes, and added to the hybridization buffer containing the Northern blots. The blots were allowed to hybridize for about two hours at about 65° C. After hybridization the blots were washed in 2×SSC for 30 minutes at room temperature, followed by 3 successive washes in 0.2×SSC containing 0.1 percent SDS at about 60° C. for 30 minutes. The blots were dried briefly and exposed to an image analyzer screen for 6 days. The results are shown in FIG. 11. TNFr/OPG-like receptor mRNA was mainly detected in peripheral blood leukocytes, spleen, testis and skeletal muscle.

What is claimed

1. An isolated nucleic acid molecule comprising a nucleotide sequence selected from the group consisting of:
(a) the nucleotide sequence set forth in SEQ ID NOS: 1 or 3;
(b) a nucleotide sequence encoding the polypeptide set forth in SEQ ID NOS: 2 or 4;
(c) a nucleotide sequence which hybridizes under moderately or highly stringent conditions to the complement of (a) or (b), wherein the encoded polypeptide has an activity of the polypeptide set forth in SEQ ID NOS: 2 or 4; and
(d) a nucleotide sequence complementary to any of (a)-(c).

2. An isolated nucleic acid molecule comprising a nucleotide sequence selected from the group consisting of:
(a) a nucleotide sequence encoding a polypeptide that is at least about 70 percent identical to the polypeptide set forth in SEQ ID NOS: 2 or 4, wherein the polypeptide has an activity of the polypeptide set forth in SEQ ID NOS: 2 or 4;
(b) a nucleotide sequence encoding an allelic variant or splice variant of the nucleotide sequence set forth in SEQ ID NOS: 1 or 3, wherein the encoded polypeptide has an activity of the polypeptide set forth in SEQ ID NOS: 2 or 4;
(c) a nucleotide sequence of SEQ ID NOS: 1 or 3; (a); or (b) encoding a polypeptide fragment of at least about 25 amino acid residues, wherein the polypeptide has an activity of the polypeptide set forth in SEQ ID NOS: 2 or 4;
(d) a nucleotide sequence of SEQ ID NOS: 1 or 3, or (a)-(c) comprising a fragment of at least about 16 nucleotides;
(e) a nucleotide sequence which hybridizes under moderately or highly stringent conditions to the complement of any of (a)-(d), wherein the polypeptide has an activity of the polypeptide set forth in SEQ ID NOS: 2 or 4; and
(f) a nucleotide sequence complementary to any of (a)-(c).

3. An isolated nucleic acid molecule comprising a nucleotide sequence selected from the group consisting of:
(a) a nucleotide sequence encoding a polypeptide set forth in SEQ ID NOS: 2 or 4 with at least one conservative amino acid substitution, wherein the polypeptide has an activity of the polypeptide set forth in SEQ ID NOS: 2 or 4;
(b) a nucleotide sequence encoding a polypeptide set forth in SEQ ID NOS: 2 or 4 with at least one amino acid insertion, wherein the polypeptide has an activity of the polypeptide set forth in SEQ ID NOS: 2 or 4;
(c) a nucleotide sequence encoding a polypeptide set forth in SEQ ID NOS: 2 or 4 with at least one amino acid deletion, wherein the polypeptide has an activity of the polypeptide set forth in SEQ ID NOS: 2 or 4;
(d) a nucleotide sequence encoding a polypeptide set forth in SEQ ID NOS: 2 or 4 which has a C- and/or N-terminal truncation, wherein the polypeptide has an activity of the polypeptide set forth in SEQ ID NOS: 2 or 4;
(e) a nucleotide sequence encoding a polypeptide set forth in SEQ ID NOS: 2 or 4 with at least one modification selected from the group consisting of amino acid substitutions, amino acid insertions, amino acid deletions, C-terminal truncation, and N-terminal truncation, wherein the polypeptide has an activity of the polypeptide set forth in SEQ ID NOS: 2 or 4;
(f) a nucleotide sequence of (a)-(e) comprising a fragment of at least 16 nucleotides;
(g) a nucleotide sequence which hybridizes under moderately or highly stringent conditions to the complement of any of (a)-(f), wherein the polypeptide has an activity of the polypeptide set forth in SEQ ID NOS: 2 or 4; and
(h) a nucleotide sequence complementary to any of (a)-(e).

4. A vector comprising the nucleic acid molecule of claims 1, 2, or 3.

5. A host cell comprising the vector of claim 4.

6. The host cell of claim 5 that is a eukaryotic cell.

7. The host cell of claim 5 that is a prokaryotic cell.

8. A process of producing a TNFr/OPG-like polypeptide comprising culturing the host cell of claim 6 under suitable conditions to express the polypeptide, and optionally isolating the polypeptide from the culture.

9. A polypeptide produced by the process of claim 8.

10. The process of claim 11, wherein the nucleic acid molecule comprises promoter DNA other than the promoter DNA for the native TNFr/OPG-like polypeptide operatively linked to the DNA encoding the TNFr/OPG-like polypeptide.

11. The isolated nucleic acid molecule according to claim 2 wherein the percent identity is determined using a computer program selected from the group consisting of GAP, BLASTP, BLASTN, FASTA, BLASTA, BLASTX, BestFit, and the Smith-Waterman algorithm.

12. A process for identifying candidate inhibitors of TNFr/OPG-like polypeptide activity or production comprising exposing a cell according to claims 6, 7, or 8 to the candidate inhibitors, and measuring TNFr/OPG-like polypeptide activity or production in said cell, comparing activity of TNFr/OPG-like in cells exposed to the candidate inhibitor with activity in cells not exposed to the candidate inhibitor.

13. A process for identifying candidate stimulators of TNFr/OPG-like polypeptide activity or production comprising exposing a cell according to claims 6, 7, or 8 to the candidate stimulators, and measuring TNFr/OPG-like polypeptide activity or production in said cell, comparing activity of TNFr/OPG-like in cells exposed to the candidate stimulator with activity in cells not exposed to the candidate stimulator.

14. An isolated polypeptide comprising the amino acid sequence set forth in SEQ ID NOS: 2 or 4.

15. An isolated polypeptide comprising the amino acid sequence selected from the group consisting of:
(a) the mature amino acid sequence set forth in SEQ ID NOS: 2 or 4, comprising a mature amino terminus at residue 1, optionally further comprising an amino-terminal methionine;
(b) an amino acid sequence for an ortholog of SEQ ID NOS: 2 or 4, wherein the encoded polypeptide has an activity of the polypeptide set forth in SEQ ID NOS: 2 or 4;
(c) an amino acid sequence that is at least about 70 percent identical to the amino acid sequence of SEQ ID NOS: 2 or 4, wherein the polypeptide has an activity of the polypeptide set forth in SEQ ID NOS: 2 or 4;
(d) a fragment of the amino acid sequence set forth in SEQ ID NOS: 2 or 4 comprising at least about 25 amino acid residues, wherein the polypeptide has an activity of the polypeptide set forth in SEQ ID NOS: 2 or 4;
(e) an amino acid sequence for an allelic variant or splice variant of either the amino acid sequence set forth in SEQ ID NOS: 2 or 4, or at least one of (a)-(c) wherein the polypeptide has an activity of the polypeptide set forth in SEQ ID NOS: 2 or 4.

16. An isolated polypeptide comprising the amino acid sequence selected from the group consisting of:
(a) the amino acid sequence set forth in SEQ ID NOS: 2 or 4 with at least one conservative amino acid substitution, wherein the polypeptide has an activity of the polypeptide set forth in SEQ ID NOS: 2 or 4;
(b) the amino acid sequence set forth in SEQ ID NOS: 3 OR 4 with at least one amino acid insertion, wherein the polypeptide has an activity of the polypeptide set forth in SEQ ID NOS: 2 or 4;
(c) the amino acid sequence set forth in SEQ ID NOS: 2 or 4 with at least one amino acid deletion, wherein the polypeptide has an activity of the polypeptide set forth in SEQ ID NOS: 2 or 4;
(d) the amino acid sequence set forth in SEQ ID NOS: 3 OR 4 which has a C- and/or N-terminal truncation, wherein the polypeptide has an activity of the polypeptide set forth in SEQ ID NOS: 2 or 4; and
(e) the amino acid sequence set forth in SEQ ID NOS: 2 or 4, with at least one modification selected from the group consisting of amino acid substitutions, amino acid insertions, amino acid deletions, C-terminal truncation, and N-terminal truncation, wherein the polypeptide has an activity of the polypeptide set forth in SEQ ID NOS: 2 or 4.

17. An ortholog of claim 15 or 16 wherein the polypeptide is encoded by the polynucleotide set forth in SEQ ID NO: 6.

18. A polypeptide according to claim 15 or 16 wherein the amino acid at position 42 of SEQ ID NOS: 2 is selected from the group consisting of glycine or proline.

19. A polypeptide according to claim 15 or 16 wherein the amino acid at position 51 of SEQ ID NOS: 2 is selected from the group consisting of serine, threonine, aspargine, glutamine and cysteine.

20. A polypeptide according to claim 15 or 16 wherein the amino acid at position 56 of SEQ ID NOS: 2 is selected from the group consisting of phenylalanine, tryptophan and tyrosine.

21. A polypeptide according to claim 15 or 16 wherein the amino acid at position 68 of SEQ ID NOS: 2 is selected from the group consisting of lysine, arginine and histadine.

22. A polypeptide according to claim 15 or 16 wherein the amino acid at position 71 of SEQ ID NOS: 2 is selected from the group consisting of cysteine, serine, threonine, asparagine and glutamine.

23. A polypeptide according to claim 15 or 16 wherein the amino acid at position 84 of SEQ ID NOS: 2 is selected from the group consisting of leucine, norleucine, isoleucine, valine, methionine, alanine, norleucine or phenylalanine.

24. A polypeptide according to claim 15 or 16 wherein the amino acid at position 87 of SEQ ID NOS: 2 is selected from the group consisting of aspartic acid or glutamic acid.

25. An isolated polypeptide encoded by the nucleic acid molecule of claims 1, 2, or 3.

26. The isolated polypeptide according to claim 15 or 16 wherein the percent identity is determined using a computer program selected from the group consisting of GAP, BLASTP, BLASTN, FASTA, BLASTA, BLASTX, BestFit, and the Smith-Waterman algorithm.

27. An antibody produced by immunizing an animal with a peptide comprising an amino acid sequence of SEQ ID NOS: 2 or 4.

28. An antibody or fragment thereof that specifically binds the polypeptide of claims 13, 14 or 15.

29. The antibody of claim 27 that is a monoclonal antibody.

30. A hybridoma that produces a monoclonal antibody that binds to a peptide comprising an amino acid sequence of SEQ ID NOS: 2 or 4.

31. A method of detecting or quantitating the amount of TNFr/OPG-like in a sample comprising contacting a sample suspected of containing TNFr/OPG-like polypeptide with the anti-TNFr/OPG-like antibody or fragment of claims 27, 28 or 29 and detecting the binding of said antibody or fragment.

32. A selective binding agent or fragment thereof that specifically binds at least one polypeptide wherein said polypeptide comprises the amino acid sequence selected from the group consisting of:
(a) the amino acid sequence set forth in SEQ ID NOS: 2 or 4;
(b) a fragment of the amino acid sequence set forth in at least one of SEQ ID NOS: 2 or 4; and
(c) a naturally occurring variant of (a) or (b).

33. The selective binding agent of claim 32 that is an antibody or fragment thereof.

34. The selective binding agent of claim 32 that is a humanized antibody.

35. The selective binding agent of claim 32 that is a human antibody or fragment thereof.

36. The selective binding agent of claim 32 that is a polyclonal antibody or fragment thereof.

37. The selective binding agent claim 32 that is a monoclonal antibody or fragment thereof.

38. The selective binding agent of claim 32 that is a chimeric antibody or fragment thereof.

39. The selective binding agent of claim 32 that is a CDR-grafted antibody or fragment thereof.

40. The selective binding agent of claim 32 that is an antiidiotypic antibody or fragment thereof.

41. The selective binding agent of claim 32 which is a variable region fragment.

42. The variable region fragment of claim 32 which is a Fab or a Fab′ fragment.

43. A selective binding agent or fragment thereof comprising at least one complementarity determining region with specificity for a polypeptide having the amino acid sequence of SEQ ID NOS: 2 or 4.

44. The selective binding agent of claim 32 which is bound to a detectable label.

45. The selective binding agent of claim 32 which antagonizes TNFr/OPG-like polypeptide biological activity.

46. A method for treating, preventing, or ameliorating a disease, condition, or disorder associated with altered levels of TNFr/OPG-like polypeptide comprising administering to a patient an effective amount of a selective binding agent according to claim 32.

47. A selective binding agent produced by immunizing an animal with a polypeptide comprising an amino acid sequence of SEQ ID NOS: 2 or 4.

48. A hybridoma that produces a selective binding agent capable of binding a polypeptide according to claims 14, 15 or 16.

49. A composition comprising the polypeptide of claims 14, 15 or 16 and a pharmaceutically acceptable formulation agent.

50. The composition of claim 49 wherein the pharmaceutically acceptable formulation agent is a carrier, adjuvant, solubilizer, stabilizer, anti-oxidant or combination thereof.

51. The composition of claim 49 wherein the polypeptide comprises the mature amino acid sequence set forth in SEQ ID NOS: 2 or 4.

52. A polypeptide comprising a derivative of the polypeptide of claims 14, 15 or 16.

53. The polypeptide of claim 52 which is covalently modified with a water-soluble polymer.

54. The polypeptide of claim 53 wherein the water-soluble polymer is selected from the group consisting of polyethylene glycol, monomethoxy-polyethylene glycol, dextran, cellulose, poly-(N-vinyl pyrrolidone) polyethylene glycol, propylene glycol homopolymers, polypropylene oxide/ethylene oxide co-polymers, polyoxyethylated polyols, and polyvinyl alcohol.

55. A composition comprising a nucleic acid molecule of claims 1, 2, or 3 and a pharmaceutically acceptable formulation agent.

56. A composition of claim 55 wherein said nucleic acid molecule is contained in a viral vector.

57. A viral vector comprising a nucleic acid molecule of claims 1, 2, or 3.

58. A fusion polypeptide comprising the polypeptide of claims 14, 15 or 16 fused to a heterologous amino acid sequence.

59. The fusion polypeptide of claim 58 wherein the heterologous amino acid sequence is an IgG constant domain or fragment thereof.

60. A method for treating, preventing or ameliorating a medical condition in a mammal resulting from decreased levels of TNFr/OPG-like polypeptide comprising administering to a patient the polypeptide of claims 14, 15 or 16 or the polypeptide encoded by the nucleic acid of claims 1, 2, or 3 to said mammal.

61. A method of diagnosing a pathological condition or a susceptibility to a pathological condition in a subject caused by or resulting from abnormal levels of TNFr/OPG-like polypeptide comprising:
(a) determining the presence or amount of expression of the polypeptide of claims 14, 15 or 16 or the polypeptide encoded by the nucleic acid molecule of claims 1, 2, or 3 in a sample; and
(b) comparing the level of TNFr/OPG-like polypeptide in a biological, tissue or cellular sample from normal subjects or the subject at an earlier time, wherein susceptibility to a pathological condition is based on the presence or amount of expression of the polypeptide.

62. A device, comprising:
(a) a membrane suitable for implantation; and
(b) cells encapsulated within said membrane, wherein said cells secrete a polypeptide of claims 14, 15 or 16, and wherein said membrane is permeable to said protein and impermeable to materials detrimental to said cells.

63. A device, comprising:
(a) a membrane suitable for implantation; and
(b) the TNFr/OPG-like polypeptide encapsulated within said membrane, wherein said membrane is permeable to the polypepetide.

64. A method of identifying a compound which binds to a polypeptide comprising:
(a) contacting the polypeptide of claims 14, 15 or 16 with a compound; and
(b) determining the extent of binding of the polypeptide to the compound.

65. A method of identifying antagonists of TNFr/OPG-like biological activity comprising:
(a) contacting a small molecule compound with a TNFr/OPG-like polypeptide;
(b) detecting the biological activity of TNFr/OPG-like in the presence of said small molecule compound; and
(c) comparing the level of TNFr/OPG-like biological activity in the presence and absence of said small molecule compound.

66. The method of claim 65 wherein the small molecule compound is a member of a naturally occurring chemical library.

67. The method of claim 65 wherein the small molecule compound is a member of a naturally occurring medicinal chemical library.

68. The method of claim 65 wherein the small molecule compound is a member of a combinational chemical library.

69. A method of identifying a polypeptide which binds to a TNFr/OPG-like polypeptide, wherein the method utilizes a yeast two-hybrid approach comprising:
(a) preparing a bait construct comprising a GAL4 DNA binding domain fused to the nucleotide sequence of claim 1, 2 or 3;
(b) screening a cDNA library with the bait construct, wherein said library consists of nucleotide sequences fused to a GAL4 activation domain, and
(c) identifying polypeptides that bind to said construct by detecting transcriptional activation of a reporter gene under control of GAL4.

70. A TNFr/OPG-like polypeptide binding partner identified by the method of claim 71.

71. A method of modulating levels of a polypeptide in an animal comprising administering to the animal the nucleic acid molecule of claims 1, 2, or 3.

72. An antagonist of TNFr/OPG-like polypeptide activity selected from the group consisting of TNFr/OPG-like selective binding agents, small molecules, antisense oligonucleotides, and peptides or derivatives thereof having specificity for TNFr/OPG-like polypeptide.

73. A method of reducing cellular production of TNFr/OPG-like, comprising transforming or transfecting cells with an antagonist according to claim 72.

74. A method according to claim 73, wherein the antagonist is an antisense reagent, said reagent comprising an oligonucleotide comprising a single stranded nucleic acid sequence capable of binding to TNFr/OPG-like mRNA.

75. A transgenic non-human mammal comprising the nucleic acid molecule of claims 1, 2, or 3.

76. A transgenic non-human mammal comprising a disruption of the nucleic acid molecule of claim 1, 2 or 3 wherein the expression of TNFr/OPG-like polypeptide is decreased.

77. A diagnostic reagent comprising a detectably labeled polynucleotide encoding the amino acid sequence set out in SEQ ID NOS: 2 or 4, or a fragment, variant or homolog thereof including allelic variants and spliced variants thereof.

78. The diagnostic reagent of claim 77, wherein said labeled polynucleotide is a first-strand cDNA.

79. A method for determine the presence of TNFr/OPG-like nucleic acids in a biological sample comprising the steps of:
(a) providing a biological sample suspected of containing TNFr/OPG-like nucleic acids;
(b) contacting the biological sample with a diagnostic reagent according to claim 84 under conditions wherein the diagnostic reagent will hybridize with TNFr/OPG-like nucleic acids contained in said biological sample;
(c) detecting hybridization between nucleic acid in the biological sample and the diagnostic reagent; and
(d) comparing the level of hybridization between the biological sample and diagnostic reagent with the level of hybridization between a known concentration of TNFr/OPG-like nucleic acid and the diagnostic reagent.

80. A method for detecting the presence of TNFr/OPG-like nucleic acids in a tissue or cellular sample comprising the steps of:
(a) providing a tissue or cellular sample suspected of containing TNFr/OPG-like nucleic acids;
(b) contacting the tissue or cellular sample with a diagnostic reagent according to claim 77 under conditions wherein the diagnostic reagent will hybridize with TNFr/OPG-like nucleic acids;
(c) detecting hybridization between TNFr/OPG-like nucleic acid in the tissue or cellular sample and the diagnostic reagent; and
(d) comparing the level of hybridization between the tissue or cellular sample and diagnostic reagent with the level of hybridization between a known concentration of TNFr/OPG-like nucleic acid and the diagnostic reagent.

81. The method of claim 86 or 87 wherein said polynucleotide molecule is DNA.

82. The method of claim 86 or 87 wherein said polynucleotide molecule is RNA.


SEQUENCE LISTING (28 sequences)

SEQ ID NO: 1 - DNA, 2638 bp, Homo sapiens
  CDS (195)..(1484)
cgggaccttcagatatcccctcccagccgagggggcttccatctaactgtttttttggtcacggttccagggccgttttagacagtggaggccttgtggggcagggtgtgaggggtgctgagcagcaggtgtggacatgtgtgtgcaccaggcctttctacctgaccgggccggcgaccaccaggggcctgaggatgaagccaagtctgctgtgccggcccctgtcctgcttccttatgctgctgccctggcctctcgccaccctgacatcaacaaccctttggcagtgcccacctggggaggagcccgacctggacccagggcagggcacattatgcaggccctgccccccaggcaccttctcagctgcatggggctccagcccatgccagccccatgcccgttgcagcctttggaggaggctggaggcccaggtgggcatggcaactcgagatacactctgtggagactgctggcctgggtggtttgggccttggggggttccccgcgttccatgtcaaccatgttcctgggcacctctgggtactcatggctgtgatgagtgggggcggcgggcccgacgtggcgtggaggtggcagcaggggccagcagcggtggtgagacacggcagcctgggaacggcacccgggcaggtggcccagaggagacagccgcccagtacgcggtcatcgccatcgtccctgtcttctgcctcatggggctgttgggcatcctggtgtgcaacctcctcaagcggaagggctaccactgcacggcgcacaaggaggtcgggcccggccctggaggtggaggcagtggaatcaaccctgcctaccggactgaggatgccaatgaggacaccattggggtcctggtgcgcttgatcacagagaagaaagagaatgctgcggccctggaggagctgctgaaagagtaccacagcaaacagctggtgcagacgagccacaggcctgtgtccaagctgccgccagcgcccccgaacgtgccacacatctgcccgcaccgccaccatctccacaccgtgcagggcctggcctcgctctctggcccctgctgctcccgctgtagccagaagaagtggcccgaggtgctgctgtcccctgaggctgtagccgccactactcctgttcccagccttctgcctaacccgaccagggttcccaaggccggggccaaggcagggcgtcagggcgagatcaccatcttgtctgtgggcaggttccgcgtggctcgaattcctgagcagcggacaagttcaatggtgtctgaggtgaagaccatcacggaggctgggccctcgtggggtgatctccctgactccccacagcctggcctcccccctgagcagcaggccctgctaggaagtggcggaagccgtacaaagtggctgaagcccccagcagagaacaaggccgaggagaaccgctatgtggtccggctaagtgagagcaacctggtcatctgaggggcggtctagtctaaggacactgcggccctgccctgggaggttccgaaggcttcctggaggaggtggagctgcagctgggactgtgaggaccgagaagcaatggcccagcagacgagacagcaaagaccaaggcctggaggtgggagcgtctgccccagtgaggaggcaggtggccggcgggcactgtgtacaggagcaggctgagccccgcccctggccctgctgccatgttgctcccctgaaggatgccccgacccccgtgcctgccctggctggatcctaggagcccacgggattctctgtatcatcagaggctgggcttggcagaggggaggggcctgtgcccgtcacccctggccccattccttggtaattagccacacccttgcctctgtacagggccctagagcagatgtgcgtccccctcctcttccagcaggtctataaagggaaggggtagcagaaagtcctgggctaggagagtgagtccctgggttctaatcttgggcacatctgtggccatcgctgggtccatttttctgactgtgaagtaaggagagacgtctcagtacccagggcctcttcagctctttgtaggttctgggctgggttgtgggggactggggagctgggctctaccatccctcccattagtagctttatccagccccgtttttgctgcttccagggcctctgccttcaaggcccccatggggctgtccatccatggctctgcctacggaaggggcttaatgcatgtgcctgcccctcccccagctgtttttaatgaaactgaaaaaatagacttgatcccggcaggactgtgatacagagccctagcctgcccagccagccccaagatctcaggagctttagggagaagacttggtggggctggagcacaccttgggcctcagtggtttctgtgtccctgtggtgccagtgcttctgggcagtgcaggcggctgccaggcccagccctgacttccactctggctcagcaacctggttatttatgtggggccgtgcaggcatgggcccactgcctgtccatcctgtttctcttatttattgaaactcaccattgccctatccttgtgtctccacccccttccatgtgttgaataataaaaggtgggaaagtgctg

SEQ ID NO: 2 - protein, 430 aa, Homo sapiens
MKPSLLCRPLSCFLMLLPWPLATLTSTTLWQCPPGEEPDLDPGQGTLCRPCPPGTFSAAWGSSPCQPHARCSLWRRLEAQVGMATRDTLCGDCWPGWFGPWGVPRVPCQPCSWAPLGTHGCDEWGRRARRGVEVAAGASSGGETRQPGNGTRAGGPEETAAQYAVIAIVPVFCLMGLLGILVCNLLKRKGYHCTAHKEVGPGPGGGGSGINPAYRTEDANEDTIGVLVRLITEKKENAAALEELLKEYHSKQLVQTSHRPVSKLPPAPPNVPHICPHRHHLHTVQGLASLSGPCCSRCSQKKWPEVLLSPEAVAATTPVPSLLPNPTRVPKAGAKAGRQGEITILSVGRFRVARIPEQRTSSMVSEVKTITEAGPSWGDLPDSPQPGLPPEQQALLGSGGSRTKWLKPPAENKAEENRYVVRLSESNLVI

SEQ ID NO: 3 - DNA, 2479 bp, Mus musculus
  CDS (91)..(1398)
caggctgcgcggccggccccgagcgctcgcctagcggggccccggcgccgcgtcggacgctgagcgaagctggtgctgcgggccaggtcaatgtcactccagggcctgatgatgaagcggaccttgctgtgctggcccctgtcttgcctctttgtgctgctgccctggcctctggccactccaacaccaataactccttggctgtgtccacctggcaaagagcctgacccagatccaggacagggcacattatgcagaacttgccccccaggaaccttttcagcctcatggaactcctatccatgccagcctcattaccgatgcagccttcaaaagaggctggaggcccaggctggcacagcaactcatgatacaatgtgtggagactgccagcatgggtggtttgggccacagggagttcctcatgttccgtgtcagccatgttccaaggcacctccaagtactggtggctgtgatgagtcagggcggcggggccggcgtggcgtcgaagtggcagcaggtaccagtagcaacggtgaacctcggcagcccgggaatggcactcgggcaggcggtcctgaggagacggctgcccagtatgcagtgattgccatcgttcctgtcttttgtctcatggggcttctgggcatcctggtgtgcaacctgctcaagcggaagggctaccattgcacagcccaaaaggaagttgggcccagccctggtggaggaggcagcgggattaatcctgcctataggactgaagatgccaacgaggacaccattggagtcctggtgcgcctgatcacagagaagaaagagaatgcagcggccctggaggagctgttgaaagaatatcacagcaaacagctggtacagacaagtcacaggcctgtacccaggctgctgccggcctcacccagcataccccacatctgcccgcatcaccaccacctgcacactgtgcagggcctggcctcactctctggcccctgttgctcccgttgtagccagaagtggccagaggtgctgctgtctcctgaggcagcagctgccaccactcctgctcccacccttctgcctactgcatccagggctcccaaggctagtgccaagccaggacgtcagggcgagattaccatcttgtctgtgggcaggttccgtgtggctcgtattcctgagcagcggaccagttcattgttatctgaggtgaagaccatcacggaggctgggccttcagagggtgatctccctgactccccacagcctggttttccccccgagcagcgggcactgctgggaagtggtgggagccatactaagtggttgaagcccccagcagagaacaaagctgaggagaaccgctatgtggtccggctaagtgaaagcaacctggtcatctgatgggctgtctagaattagacactctgccctgtcctgggaggttctgaaggcttcctgcaggagggagagctgcagctgggactgaggaccaagatgcaaaggccaagtcctggaggtgggaccgtccgccccactgaggaggcagcctgcggcacagcacgtgagcaggagatcaagagcccaccctatccctgcagtcccggttacttccatgcagggtgctgtaaccctgtgcctgccctgaacacatcataggagccctctgtcccttagaggtctggtttggtggaggagtggtatctgtacctggccccaagcttgtgccttgggaactagccactcttgcccatgtcctggaccctggatgtgactccctctcttctggcaggccctatagagggaaggggtagcaaagagccctgtactggtggcagagtacctgggttccaatcctgggcttatccctaggtacgtaggggaggagaactcagttcccaggacctctccagctctttgcagattctgggctgagtcctgttggggggagcttgactttgctaccctcccattagtagctttatctggcctgtttttgctgcttcctgggccttggccttcatggctcccatgggactgtctattatggtgatgccttcagaaggggtttaatgcatgtgcctgcccctaccctgctatttttaatgaaactgaaaaatgacttgacttggacagggctctctggtgcagagcctcagtccaccctgctgccctcaagctctggagctgtgggaagaggagacaggcaggctagggagtgcctgtggcctgtggttttcaatgcccctgtggtacagtatctgcctgagttttgggtagcaggggtgactgccaatccagcctgtcttagtctctgctctggctcagtgcctcgttatttatgtggggccgtgcaggcgcggggcccactgcccatcccatttcttatttattgaaacctgctgttgccctgcccctacatctccagccccacacacttgagtaataaaaggtggaaaatgtcaaaaaaaaaaaaaaaagg

SEQ ID NO: 4 - protein, 436 aa, Mus musculus
MSLQGLMMKRTLLCWPLSCLFVLLPWPLATPTPITPWLCPPGKEPDPDPGQGTLCRTCPPGTFSASWNSYPCQPHYRCSLQKRLEAQAGTATHDTMCGDCQHGWFGPQGVPHVPCQPCSKAPPSTGGCDESGRRGRRGVEVAAGTSSNGEPRQPGNGTRAGGPEETAAQYAVIAIVPVFCLMGLLGILVCNLLKRKGYHCTAQKEVGPSPGGGGSGINPAYRTEDANEDTIGVLVRLITEKKENAAALEELLKEYHSKQLVQTSHRPVPRLLPASPSIPHICPHHHHLHTVQGLASLSGPCCSRCSQKWPEVLLSPEAAAATTPAPTLLPTASRAPKASAKPGRQGEITILSVGRFRVARIPEQRTSSLLSEVKTITEAGPSEGDLPDSPQPGFPPEQRALLGSGGSHTKWLKPPAENKAEENRYVVRLSESNLVI

SEQ ID NO: 5 - DNA, 482 bp, Homo sapiens
ctgtcctgggagggccctgagggccaggggcagagtcctgtgcctggcccccaagggtcctcaggcttggctcctggccatgctctcaccctttacctcccacaggacccagggcagggcacattatgcaggccctgccccccaggcaccttctcagctgcatggggctccagcccatgccagccccatgcccgttgcagcctttggaggaggctggaggcccaggtgggcatggcaactcgagatacactctgtggagactgctggcctgggtaagccaaagggagtgcggggagggctcctggctgggtgaccaggactctggatcctggggccccagccttattgtaccctgagcaggcctcattcttcccatctgtgaaatgggatggggcaggaccacggagggtgcctggtaggaaggaatccagcctctcctaaggatagtgtttggggaaacttctgggcctcagtggtatctt

SEQ ID NO: 6 - protein, 56 aa, Homo sapiens
DPGQGTLCRPCPPGTFSAAWGSSPCQPHARCSLWRRLEAQVGMATRDTLCGDCWPG

SEQ ID NO: 7 - protein, 91 aa, Homo sapiens
LSWEGPEGQGQSPVPGPQGSSGLAPGHALTLYLPQDPGQGTLCRPCPPGTFSAAWGSSPCQPHARCSLWRRLEAQVGMATRDTLCGDCWPG

SEQ ID NO: 8 - protein, 120 aa, Homo sapiens
CNRTHNRVCECKEGRYLEIEFCLKHRSCPPGFGVVQAGTPERNTVCKRCPDGFFSNETSSKAPCRKHTNCSVFGLLLTQKGNATHDNICSGNSESTQKCGIDVTLCEEAFFRFAVPTKFT

SEQ ID NO: 9 - DNA, 22 bp, Artificial Sequence
  Description of Artificial Sequence PCR primer 2374-51
ccccaggcaccttctcagctgc

SEQ ID NO: 10 - DNA, 23 bp, Artificial Sequence
  Description of Artificial Sequence PCR PRimer 2374-52
gtgtatctcgagttgccatgccc

SEQ ID NO: 11 - DNA, 23 bp, Artificial Sequence
  Description of Artificial Sequence PCR Primer 870-02
agcggataacaatttcacacagg

SEQ ID NO: 12 - DNA, 29 bp, Artificial Sequence
  Description of Artificial Sequence PCR Primer 1916-83
ggctcgtatgttgtgtggaattgtgagcg

SEQ ID NO: 13 - DNA, 22 bp, Artificial Sequence
  Description of Artificial SequencePCR primer 2374-53
cccaggccagcagtctccacag

SEQ ID NO: 14 - DNA, 24 bp, Artificial Sequence
  Description of Artificial Sequence PCR Primer 1019-06
gctctaatacgactcactataggg

SEQ ID NO: 15 - DNA, 29 bp, Artificial Sequence
  Description of Artificial Sequence PCR Primer 1916-82
catgattacgccaagctctaatacgactc

SEQ ID NO: 16 - DNA, 23 bp, Artificial Sequence
  Description of Artificial Sequence PCR Primer 1340-35
cccagtcacgacgttgtaaaacg

SEQ ID NO: 17 - DNA, 26 bp, Artificial Sequence
  Description of Artificial Sequence PCR Primer 1019-05
tgaatttaggtgacactatagaagag

SEQ ID NO: 18 - DNA, 20 bp, Artificial Sequence
  Description of Artificial Sequence PCR Primer 1019-05
gcccgttgcagcctttggag

SEQ ID NO: 19 - DNA, 21 bp, Artificial Sequence
  Description of Artificial Sequence PCR primer
tccaccaccctgttgctgtag

SEQ ID NO: 20 - DNA, 24 bp, Artificial Sequence
  Description of Artificial Sequence PCR Primer
gaccacacagtccatgccatcact

SEQ ID NO: 21 - DNA, 30 bp, Artificial Sequence
  Description of Artificial Sequence PCR Primer
ccatcgatggctgagcagcaggtgtggaca

SEQ ID NO: 22 - DNA, 24 bp, Artificial Sequence
  Description of Artificial Sequence PCR Primer
tggcgatgacggtgacctgggcgg

SEQ ID NO: 23 - protein, 12 aa, Artificial Sequence
  Description of Artificial Sequence Peptide
STTLWQCPPGEE

SEQ ID NO: 24 - protein, 14 aa, Artificial Sequence
  Description of Artificial Sequence Peptide
GVEVAAGASSGGET

SEQ ID NO: 25 - protein, 9 aa, Artificial Sequence
  Description of Artificial Sequence Furin cleavage site
RRARRGVEV

SEQ ID NO: 26 - protein, 9 aa, Artificial Sequence
  Description of Artificial Sequence FLAG epitope sequence
MDYKDDDDK

SEQ ID NO: 27 - protein, 11 aa, Artificial Sequence
  Description of Artificial Sequence Peptide
GRKKKRRQRRR

SEQ ID NO: 28 - protein, 15 aa, Artificial Sequence
  Description of Artificial Sequence Peptide
GGGGYGRKKRRQRRR